(19) United States
(12) Patent Application Publication
(10) Pub. No.: US 2025/0136652 A1
(43) Pub. Date: May 1, 2025

(54) FUSION PROTEIN FOR TARGETING RECOMBINANT PROTEINS FOR DEGRADATION

(71) Applicant: The Regents of the University of California, Oakland, CA (US)

(72) Inventors: Matthew KIM, San Francisco, CA (US); Hana EL-SAMAD, San Francisco, CA (US); Andrew H. NG, San Francisco, CA (US)

(21) Appl. No.: 18/835,327

(22) PCT Filed: Mar. 1, 2023
(86) PCT No.: PCT/US2023/063471

§ 371 Date: Aug. 1, 2024

Related U.S. Application Data
(60) Provisional application No. 63/315,925, filed on Mar. 2, 2022.

(51) Int. Cl. C07K 14/47; C07K 5/11; C07K 7/06; C12N 5/0783; C12N 15/86

(52) U.S. Cl. CPC C07K 14/4702; C07K 5/1019; C07K 7/06; C12N 5/0636; C12N 15/86; C07K 2319/03; C07K 2319/73; C07K 2319/80; C07K 2319/95; C12N 2740/16043

(57) ABSTRACT

Described herein, among other things, is a fusion protein for degrading a target protein in trans. The fusion protein may comprise a lysine-free alpha-helical heterodimerization domain and a C-terminal degron. Degrons are believed to act as a degradation signal that recruits the proteosome to the target protein, thereby degrading the protein. A nucleic acid encoding the same and a cell containing the nucleic acid and a target protein are also described.

### CROSS-REFERENCING

This application claims the benefit of U.S. provisional application Ser. No. 63/315,925, filed on Mar. 2, 2022, which application is incorporated by reference in its entirety.

### GOVERNMENT RIGHTS

This invention was made with government support under grant no. HR0011-16-2-0045 awarded by Defense Advanced Research Projects Agency. The government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, “UCSF-672WO_SEQ_LIST”, created on Feb. 17, 2023, and having a size of 381,668 bytes. The contents of the Sequence Listing XML are incorporated herein by reference in their entirety.

### BACKGROUND

Although some cell therapies have demonstrated remarkable therapeutic responses and benefits for patients with some diseases, development of effective cell-based therapies for other diseases remains a challenge, in large part due to the difficulty in regulating gene expression in the cells. This problem is exacerbated in immune cell-based therapies because many of those therapies are extremely potent and need to be “turned off” after a certain amount of time or if off-site effects are observed.

This disclosure provides a solution to this problem.

### SUMMARY

Provided herein, among other things, is a fusion protein for degrading a target protein in trans. The fusion protein may comprise a lysine-free alpha-helical heterodimerization domain and a C-terminal degron. Degrons are believed to act as a degradation signal that recruits the proteosome. Binding of the fusion protein to a target protein that comprises a binding partner for the lysine-free alpha-helical heterodimerization domain induces degradation of the target protein in trans, meaning that fusion protein targets other proteins for degradation. In order to avoid cis degradation (at least until the fusion protein has bound to its partner), the intracellular part of the fusion protein (which may be all of the protein if it is expressed as a soluble protein), including the heterodimerization domain is engineered to be lysine-free, which allows it to avoid ubiquitinated and subsequently degraded by the proteasome until it binds to its target. Depending on how the fusion protein is designed, the heterodimerization domain may be 30-80 amino acids in length and, in some embodiments, the entire fusion protein may be no more than 200 amino acids in length (e.g., 40-100 amino acids). The fusion protein is relatively small, it binds specifically binds to the target protein (i.e., and not to other proteins in the cell) and has a potent ability to degrade other proteins. These features make it an ideal system for eliminating proteins in therapeutic cells in a controllable way.

12. The fusion protein of any one of claims 1-11, wherein the fusion partner comprises amino acids 1-41 of or all of SEQ ID NO: 413.

A recombinant nucleic acid containing a promoter and a coding sequence for the fusion protein are also described, where the promoter may be chemically-inducible, tissue-specific, cell state-responsive, or activated by an external stimulus that is recognized at the plasma membrane, for example.

A cell comprising the nucleic acid is also provided. As would be apparent, the cell may further comprise a target protein that comprise a binding partner for the lysine-free alpha-helical heterodimerization domain. The target protein may be a recombinant transcription factor, enzyme, kinase, receptor or a cytokine, for example.

A method for degrading a target protein and a method of treatment are also provided.

These and other advantages may be become apparent in view of the following discussion.

### BRIEF DESCRIPTION OF THE FIGURES

The skilled artisan will understand that the drawings, described below, are for illustration purposes only. The drawings are not intended to limit the scope of the present teachings in any way.

FIG. 1: Schematically illustration of an example of the present fusion protein, a nucleic acid encoding the fusion protein and a cell containing the same and a target protein.

FIG. 2A: Left: Cartoon depiction of truncated ubiquitin proteasome pathway (UPP). Right: Cartoon of example of Synthetic Targeter of Ubiquitination and Degradation (“STUD”) bridging a target protein of interest with the endogenous UPP to initiate degradation of the target.

FIG. 2B: Top: Cartoon depiction of plasmids used in demonstration of STUD-induced degradation of green fluorescent protein (GFP) target in Jurkat T cells. Jurkat cells were lentivirally transduced with two plasmids. The first encodes a STUD, or control with a mutated degron, and a mCherry transduction marker separated by a 2A element and the second encodes GFP target protein and a BFP transduction marker separated by a 2A element. In the case of the SynZip STUD, the GFP target is fused to a heterodimeric SynZip protein complementary to the binding domain on the STUD. These transduced cells are analyzed for fluorescence by flow cytometry 48 hours after removal of virus. Cells are first gated on these transduction markers to isolate relevant populations. Then, normalized GFP fluorescence is calculated by normalizing each cell's GFP fluorescence, as obtained by flow cytometry, by its BFP fluorescence. Then, the median normalized GFP fluorescence of each flow cytometry distribution is calculated and shown in the bar plot. Each dot represents the mean of the median normalized GFP fluorescence of three technical replicates in three independent experiments. The flow cytometry distributions of one of these technical replicates is shown below. In the distributions, the “+TRGN” (SEQ ID NO:50) condition corresponds to the “RRRG−” (SEQ ID NO:32) condition in the bar plot. Error bars represent the standard deviation.

FIG. 2C: STUD degradation of a GFP target in various mammalian cell lines. Median normalized GFP values are calculated as in FIG. 2B, but each dot here represents a technical replicate. Error bars represent standard deviation.

FIG. 3: STUD degradation of GFP is mediated by the cullins in UPP. Representative flow cytometry distributions of GFP fluorescence of Jurkat T cells expressing the SynZip STUD described in FIG. 2B treated with one of three drugs or a DMSO vehicle control. Distributions are representative of three independent experiments.

FIG. 4A: Tethering of STUD to plasma membrane allows for functional knockdown for second-generation chimeric antigen receptors (CAR). Cartoon diagram of membrane tethered STUD (‘memSTUD’) and non-functional (‘NF’) control relative to original ‘soluble’ STUD design. The DNA cartoon represents the plasmid used in experiments done in this panel. (RRRG (SEQ ID NO:32); TRGN (SEQ ID NO:50)).

FIG. 4B: Plasmid diagramed in FIG. 4A transduced into Jurkat T cells and CAR fluorescence measured 72 hours removal of lentivirus. CAR fluorescence is measured by antibody stain for myc tag fused to CAR extracellular domain. Bar plot displays the median fluorescence based on antibody stain signal and representative flow cytometry distributions show. Dots represent three independent experiments and errors bars show standard deviation.

FIG. 4C: Plasmid in FIG. 4A is transduced into CD8+ primary T cells and anti-HER2 4-1BB CD3zeta CAR expression and activation is assayed. Left: Representative flow cytometry distributions of anti-HER2 4-1BB CD3zeta CAR fluorescence by antibody stain. Middle: Engineered T cells are cocultured with K562 target cells expressing various levels of HER2 antigen for 72 hours and lysis is measured by flow cytometry. Lysis is calculated relative to lysis observed when UnT cells are cocultured with target cells. Right: Median fluorescence of T cell activation marker CD25 after coculture is measured by antibody stain for CD25 and flow cytometry.

FIG. 4D. Plasmid in FIG. 4A is transduced into CD8+ primary T cells and anti-CD19 4-1BB CD3zeta CAR expression and activation is assayed. Left: Cullin inhibitor MLN4924 is added to engineered T cells to rescue degradation of CAR by STUD. Bar plot shows CAR fluorescence by antibody stain and flow cytometry after 5 hours of incubation with inhibitor. Black bars represent each condition with DMSO vehicle control. Middle: Engineered T cells are cocultured with NALM6 target cells for 72 hours and lysis is measured by flow cytometry. Lysis is calculated relative to lysis of NALM6 cells cultured with UnT T cells. Right: Median fluorescence of T cell activation marker CD25 after coculture is measured by antibody stain for CD25 and flow cytometry.

FIG. 5A: Design of new synthetic receptor allows for antigen triggered degradation of cytosolic proteins. Cartoon diagram of novel NotchSTUD synthetic receptor that couples antigen binding to target degradation.

FIG. 5B: Left: Representative flow cytometry distribution of target GFP fluorescence of cells gated on mCherry and tagBFP co-transduction markers 72 hours after coculture. Right: Quantification of median GFP target fluorescence normalized by tagBFP co-transduction marker following 72 hours of coculture with HER2+ K562 target cells or WT K562 target cells. Black bars on bar plot represent standard deviation of three technical triplicates.

FIG. 6A: STUDs can be composed into negative feedback circuit to regulate synthetic transcription factor (SynTF). Cartoon diagram of plasmids used in negative feedback circuit transduced into Jurkat T cells. Synthetic transcription factors used in this work (grey) are made up of a transcriptional activation domain (AD) and a DNA binding domain (DBD) separated by a NS3 protease. In the absence of the NS3 inhibitor grazoprevir (GZV), the TF is destabilized and non-functional. On the other hand, the addition of GZV stabilizes the TF and allows for transcription of the gene cassette downstream of the SynTF promoter (pSynTF). Three types of circuits were designed: (1) A negative feedback circuit that has the SynTF driving expression of the GFP reporter and the STUD which results in degradation of the SynTF and shut off the circuit. (2) an open loop control where there is no STUD present to gauge the maximum activity of the SynTF and (3) an open loop control where the STUD is constitutively present and degrading the SynTF.

FIG. 6B: Dose response of circuit after 72 hours of incubation with GZV at 37 C. The plot displays GZV concentration versus Fold change of the median GFP relative to the DMSO vehicle control.

FIG. 7: Dose dependent degradation of STUDs allow for use as ON switch CAR. We engineer CD8+ primary human T cells that express an antiCD19BBz CAR fused to a SynZip and a STUD to recognizes the SynZip. As a control, the CD8+ primary human T cells that express the same CAR without the SynZip and the SynZip STUD were also engineered. Both these cell lines were cocultured and an untransduced (UnT) control with NALM6 target cells for 72 hours at 37 C in the presence of various concentrations of MLN4924. Lysis of target cells was calculated by each line relative to NALM6 cells cultured alone by flow cytometry.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein. and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F (ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and-binding site. This region consists of a dimer of one heavy-and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, the first member of a specific binding pair present in the extracellular domain of a chimeric Notch receptor polypeptide of the present disclosure binds specifically to a second member of the specific binding pair. “Specific binding” refers to binding with an affinity of at least about 10−7 M or greater, e.g., 5×10−7 M, 10−8 M, 5×10−8 M, and greater. “Non-specific binding” refers to binding with an affinity of less than about 10−7 M, e.g., binding with an affinity of 10−6 M, 10−5 M, 10−4 M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013);5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

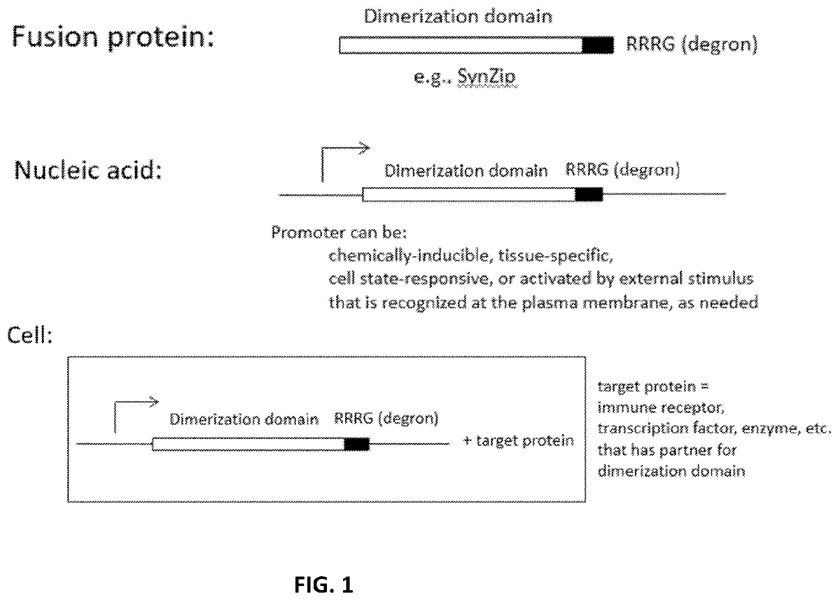

As noted above, this disclosure provides, among other things, a fusion protein for degrading a target protein in trans. The fusion protein may comprise a lysine-free alpha-helical heterodimerization domain and a C-terminal degron. Degrons are believed to act as a degradation signal that recruits the proteosome to the target protein, thereby degrading the target protein in trans. A nucleic acid encoding the same and a cell containing the nucleic acid and a target protein are also described. FIG. 1 schematically illustrates an example of the present fusion protein, a nucleic acid encoding the same and a cell containing the same and a target protein.

In any embodiment, the heterodimerization domain may be relatively short (e.g., 28-80 amino acids in length). In some embodiments, the heterodimerization domain may be composed of a single bZIP domain (which are typically 28-50 in length) or a “designed heterodimer” (which are typically longer at 50-80 amino acids in length). Examples of synthetic bZIP domains are described in Thompson et al (ACS Synth Biol. 2012 1: 118-129) and designed heterodimers are designed in Chen et al (Nature 2019 565: 106-111) and US20210355175A1). Either way, the heterodimerization domain will contain one or two idealized alpha helix structures which each have at least 4 (e.g., at least 5, at least 6, at least 7 or at least 8) helical turns. In the case of a bZIP, the helix will contain a leucine residue (or similar) at every seventh position (i.e., at “d” position of the heptad). Designed heterodimers, which are composed of a folded alpha-helical, can be designed in silico (see Chen et al (Nature 2019 565: 106-111) and US20210355175A1).

As noted above, the heterodimerization domain should be lysine free. In some embodiments, the heterodimerization domain may be readily designed using any the known pairs of heterodimerization domains by substituting any lysines (e.g., 1, 2 or 3 or more lysines) to another amino acid, e.g., a similar amino acid such as arginine (or histidine) and making other adjustments. Because alpha helical heterodimerization domains are structurally well characterized and many modeling programs exist, such changes should be predictable.

The alpha helical heterodimerization domain in the fusion protein heterodimerizes with a corresponding alpha helical domain in the target protein, meaning that it binds to another alpha helical domain in the target protein but not to itself. Examples of alpha helical heterodimerization domains include bZIP domains (leucine zipper domains) and designed heterodimers.

The leucine zipper motif is one of the most common domains in transcription factors. The motif is found in thousands of DNA-binding proteins, including well studied transcription factors such as C/EBP, Jun, Fos, GCN4, VBP, HSF and many others. This motif has been well studied. bZIP domains that can heterodimerize can be readily designed (see, e.g., Mol et al, Protein Sci. 2001 10: 649-655 and Mason et al PNAS 2006 103 8989-8994) and the sequences of many pairs of bZIP domains are known or can be readily derived from the literature. Heterodimerizing helix-loop-helix domains (which are composed of two or more alpha helices) can be readily designed and several examples exist.

In some embodiments, alpha helical domains may be a synthetic leucine zipper domain (or “synZIPs”) (see, e.g., Potapov et al (PLoS Comput Biol 11(2): e1004046) and Thompson et al. (ACS Synth Biol. 2012 1: 118-129)). These heterodimerization domains have a coiled-coil interaction domain. Such domains have also been well characterized in a number of publication such as O'shea et al. (Curr Biol. 1993 Oct. 1;3(10):658-67), Litowski et al. J Biol Chem. 2002 Oct. 4;277(40):37272-9), Thomas et al. (J Am Chem Soc. 2013 Apr. 3;135(13):5161-6), Chen et al. (Nature. 2019 January;565(7737):106-111. doi: 10.1038/s41586-018-0802-y), Tripet et al. (Protein Eng. 1997 March; 10(3):299), Archarya et al. (Biochemistry. 2002 Dec. 3;41(48):14122-31), Archarya et al. (Biochemistry. 2006 Sep. 26;45(38):11324-32), Yu et al. (Nat Commun. 2020 Sep. 8;11(1):4476), Thompson et al. (ACS Synth Biol. 2012 Apr. 20;1(4):118-29), Holmstrom et al. (Langmuir. 2008 Oct. 21;24(20):11778-83), Papapostolou et al. (J Am Chem Soc. 2008 Apr. 16;130(15):5124-30), Stolz et al. (Viruses. 2020 Jul. 14;12(7):757), each of which is incorporated by reference herein.

In some embodiments, the heterodimerization domain may comprise a sequence that is at least 80% identical (e.g., at least 90% identical, or at least 95% identical) to at least 28, 35 or 42 contiguous amino acids of any of the sequences in Table 1 below, excluding any lysine residues. In these embodiments, the lysine residues in the sequences of Table 1 may be substituted with an arginine residue. In other words, the heterodimerization domain in the fusion protein may be shorter than a selected sequences in Table 1 (e.g., by up to 5 amino acids or up to 10 amino acids), may be lysine-free, and may have a similar amino acid sequence (e.g., at least 80% identical, at least 90% identical, or at least 95% identical) to any of the sequences, excluding lysines.

The sequences of examples of alpha-helical heterodimerization domains are listed below. In incorporating the sequences into a fusion protein, the lysines in these sequences should be changed to another amino acid, e.g., arginine.

TABLE 1

| name | sequence |
|---|---|
| SYNZIP1 | NLVAQLENEVASLENENETLKKKNLHKKDLIAYLEKEIANLRKKIEE |
| | (SEQ ID NO: 1) |
| SYNZIP2 | ARNAYLRKKIARLKKDNLQLERDEQNLEKIIANLRDEIARLENEVASHEQ |
| | (SEQ ID NO: 2) |
| SYNZIP3 | NEVTTLENDAAFIENENAYLEKEIARLRKEKAALRNRLAHKK (SEQ ID |
| | NO: 3) |
| SYNZIP4 | QKVAELKNRVAVKLNRNEQLKNKVEELKNRNAYLKNELATLENEVARLEND |
| | VAE (SEQ ID NO: 4) |
| SYNZIP5 | NTVKELKNYIQELEERNAELKNLKEHLKFAKAELEFELAAHKFE (SEQ |
| | ID NO: 5) |
| SYNZIP6 | QKVAQLKNRVAYKLKENAKLENIVARLENDNANLEKDIANLEKDIANLERD |
| | VAR (SEQ ID NO: 6) |
| SYNZIP7 | KEIEYLEKEIERLKDLREHLKQDNAAHRQELNALRLEEAKLEFILAHLLST |
| | (SEQ ID NO: 7) |
| SYNZIP8 | KEIANLEKEIASLEKKVAVLKQRNAAHKQEVAALRKEIAYVEDEIQYVEDE |
| | (SEQ ID NO: 8) |
| SYNZIP9 | QKVESLKQKIEELKQRKAQLKNDIANLEKEIAYAET (SEQ ID NO: 9) |
| SYNZIP10 | NLLATLRSTAAVLENENHVLEKEKEKLRKEKEQLLNKLEAYK (SEQ ID |
| | NO: 10) |
| SYNZIP11 | ELTDELKNKKEALRKDNAALLNELASLENEIANLEKEIAYFK (SEQ ID |
| | NO: 11) |
| SYNZIP12 | NEDLVLENRLAALRNENAALENDLARLEKEIAYLEKEIEREK (SEQ ID |
| | NO: 12) |
| SYNZIP13 | QKVEELKNKIAELENRNAVKKNRVAHLKQEIAYLKDELAAHEFE (SEQ |
| | ID NO: 13) |
| SYNZIP14 | NDLDAYEREAEKLEKKNEVLRNRLAALENELATLRQEVASMKQELQS |
| | (SEQ ID NO: 14) |
| SYNZIP15 | FENVTHEFILATLENENAKLRRLEAKLERELARLRNEVAWL (SEQ ID |
| | NO: 15) |
| SYNZIP16 | NILASLENKKEELKKLNAHLLKEIENLEKEIANLEKEIAYFK (SEQ ID |
| | NO: 16) |
| SYNZIP17 | NEKEELKSKKAELRNRIEQLKQKREQLKQKIANLRKEIEAYK (SEQ ID |
| | NO: 17) |
| SYNZIP18 | SIAATLENDLARLENENARLEKDIANLERDLAKLEREEAYF (SEQ ID |
| | NO: 18) |
| SYNZIP19 | NELESLENKKEELKNRNEELKQKREQLKQKLAALRNKLDAYKNRL (SEQ |
| | ID NO: 19) |
| SYNZIP20 | STVEELLRAIQELEKRNAELKNRKEELKNLVAHLRQELAAHKYE (SEQ |
| | ID NO: 20) |
| SYNZIP21 | NEVAQLENDVAVIENENAYLEKEIARLRKEIAALRDRLAHKK (SEQ ID |
| | NO: 21) |
| SYNZIP 22 | KRIAYLRKKIAALKKDNANLEKDIANLENEIERLIKEIKTLENEVASHEQ |
| | (SEQ ID NO: 22) |
| bZIP c-Jun | ERIARLEEKVKILKAQNSELASTANMLREQVAQLKQ (SEQ ID NO: 23) |
| bZIP JUNB | ERIARLEDKVKTLKAENAGLSSTAGLLREQVAQLKQ (SEQ ID NO: 24) |
| bZIP Fos | ELTDTLQAETDQLEDEKSALQTEIANLLKEKEKLEF (SEQ ID NO: 25) |
| bZIP ATF3 | EKTECLQKESEKLESVNAELKAQIEELKNEKQHLIY (SEQ ID NO: 26) |
| bZIP ATF4 | AECEALTGECKELEKKNEALKERADSLAKEIQYLKD (SEQ ID NO: 27) |
| bZIP ATF5 | AEGEALEGECQGLEARNRELKERAESVEREIQYVKD (SEQ ID NO: 28) |
| bZIP C/EBPA | QRNVETQQKVLELTSDNDRLRKRVEQLSRELDTLRGIFRQLPE (SEQ ID |
| | NO: 29) |
| bZIP C/EBPB | MRNLETQHKVLELTAENERLQKKVEQLSRELSTLRNLFKQLPE (SEQ ID |
| | NO: 30) |
| bZIP C/EBPG | QKAQDTLQRVNQLKEENERLEAKIKLLTKELSVLKDLFLEHAH (SEQ ID |
| | NO: 31) |
| bZIP C/EBPE | RRILETQQKVLEYMAENERLRSRVEQLTQELDTLRNLFRQIPE (SEQ ID |
| | NO: 33) |
| bZIP CHOP | RKVAQLAEENERLKQEIERLTR (SEQ ID NO: 34) |
| bZIP CREB | EYVKCLENRVAVLENQNKTLIEELKALKD (SEQ ID NO: 35) |
| bZIP NEIL3 | LNDLVLENKLIALGEENATLKAELLSLKL (SEQ ID NO: 36) |
| bZIP BATF | QKADTLHLESEDLEKQNAALRKEIKQLTE (SEQ ID NO: 37) |
| bZIP BATF2 | DKADALHQQHESLEKDNLALRKEIQSLQA (SEQ ID NO: 38) |
| bZip BATF3 | QKADKLHEEYESLEQENTMLRREIGKLTEELKHLTE (SEQ ID NO: 39) |
| bZIP HLF | RDARRLKENQIAIRASFLEKENSALRQEVADLRK (SEQ ID NO: 40) |
| bZIP MAFB | QQKHHLENEKTQLIQQVEQLKQEVSRLAR (SEQ ID NO: 41) |
| DHD1 | GSPKEEARELIRKQKELIKEQKKLIKEAKQKSDSRDAERIWKRSREINRES |
| | KKINKRIKELIKS (SEQ ID NO: 42) |
| DHD2 | PKKEAEELAEESEELHDRSEKLHERAEQSSNSEEARKILEDIERISERIEE |
| | ISDRIERLLRS (SEQ ID NO: 43) |
| DHD3 | GTKEDILERQRKIIERAQEIHRRQQEILEELERIIRKPGSSEEAMKRMLKL |
| | LEESLRLLKELLELSEESAQLLYEQR (SEQ ID NO: 44) |
| DHD4 | GTEKRLLEEAERAHREQKEIIKKAQELHRRLEEIVRQSGSSEEAKKEAKKI |
| | LEEIRELSKRSLELLREILYLSQEQKGSLVPR (SEQ ID NO: 45) |
| DHD5 | TKEDILERQRKIIERAQEIIRRQQEILEELERIIRKPGSSEEAMKRMLKLL |
| | EESLRLLKELLELLEESAQLLYEQR (SEQ ID NO: 46) |
| DHD6 | GSTEKRLLEEAERAHREAKEIIKKAQELHRRLEEIVRQSGSSEEAKKEAKK |
| | ILEEIRELSKRLLELLREILYLSQEQK (SEQ ID NO: 47) |
| DHD7 | TKEDILERARKIIERAQEIHRRQQEILEELERIIRKPGSSEEAMKRMLKLL |
| | EESLRLLKELLELSEELAQLLYEQR (SEQ ID NO: 48) |
| DHD8 | GSTEKRLLEEAERAIREQKEIIKKAQELHRRLEEIVRQSGSSEEAKKEAKK |
| | ILEEIRELSKRSLELLREILYLLQEQK (SEQ ID NO: 49) |
| DHD9 | TKEDILERQRKIIERAQEIHRRQQEILEELEYIIR (SEQ ID NO: 51) |
| DHD10 | MSEEAMKRMLKLLEESLRLLKELLELSEESAQLLYEQRKANNGSETEKRLL |
| | EEAERAHREQKEIIKKAQELHRRLEEIVRQSGSSEEAKKEAKKILEEIREL |
| | SKRSLELLREILYLSQEQK (SEQ ID NO: 52) |
| DHD11 | MTKEDILERQRKIIERAQEIHRRQQEILKEQEKIIRKPGSSEEAMKRSLKL |
| | IEESLRLLKELLELSEESAQLLYEQR (SEQ ID NO: 53) |
| DHD12 | GTEKRLLEEAERAHREQKEIIKKAQELHKELTKIHQQSGSSEEAKKRALKI |
| | SQEIRELSKRSLELLREILYLSQEQK (SEQ ID NO: 54) |
| DHD13 | TKEDILERQRKIIERAQEIHRRQQEILKRSEEIIRKPGSSEEALETLRELQ |
| | EESLRLLKELLELSEESAQLLYEQR (SEQ ID NO: 55) |
| DHD1L | GSTEKRLLEEAERAHREQKEIIKKAQELHRRTEEIIRQSGSSEEAKDELRR |
| | IQEEIRELSKRSLELLREILYLSQEQK (SEQ ID NO: 56) |
| DHD15 | TTKRYLEEAERAHREQKEIIKKAQELHRRLEEIVRQ (SEQ ID NO: 57) |
| DHD16 | GSSEEAKKEAKKILEEIRELSKRSLELLREILYLSQQVNDVDEKALERQRK |
| | IIERAQEIHRRQQEILEELERIIRKPGSSEEAMKRMLKLLEESLRLLKELL |
| | ELSEESAQLLYEAR (SEQ ID NO: 58) |
| DHD17 | EAMKRMLKLLEESLRLLKELLELSEESAQLLYEAR (SEQ ID NO: 59) |
| DHD18 | TTKRYLEEAERAHREQKEIIKKAQELHRRLEEIVRQSGSSEEAKKEAKKIL |
| | EEIRELSKRSLELLREILYLSQQVNDVDEKALERQRKIIERAQEIHRRQQE |
| | ILEELERIRKPGS (SEQ ID NO: 60) |
| DHD19 | TREELLRENIELAKEHIEIMREILELLQKMEELLEKARGADEDVAKTIKEL |
| | LRRLKEIZERNQRIAKEHEYIARERS (SEQ ID NO: 61) |
| DHD20 | GTERKLLERSRRLQEESKRLLDEMAEIMRRIKKLLKKARGADEKVLDELRK |
| | IIERIRELLDRSRKIHERSEEIAYKEE (SEQ ID NO: 62) |
| DHD21 | GDRQELIRRNIELLKEHIKILEEISQLIEELSELLDKSSSEEVVKRYKKIL |
| | ERYKQLLRKSQEIHKESSEIAKKES (SEQ ID NO: 63) |
| DHD22 | GDEQKLIERSQRMQKESLELLKEIIKILDTIEKLLDKPDSEELLDTIKKLH |
| | DTLKKIHDRNKKLLKEHEEILRQRSGSLVPR (SEQ ID NO: 64) |
| DHD23 | DKEEEYKRLLDEIKEILKESKEVLKDSKRVLEDIKRKVPDDDLVKLLEKHV |
| | RLLEEHVKLLEQLIREAEKSSK (SEQ ID NO: 65) |
| DHD24 | QGSSAEELLKKIKESEKKIRDSLRKIKEIIKKSRKEGVDDKQLDLIRKVVE |
| | SHRDLLRLHRDLLRLLREETS (SEQ ID NO: 66) |
| DHD25 | DIDESIKEVEKLLEEVEQSLQKLDDSLKKLLEKVNQDPDVDDSVRKIVKRH |
| | VEILKRHEEVLKRLIEVVKEHTKTVK (SEQ ID NO: 67) |
| DHD26 | GSDREEVHKEIVKLIREIIKIHKKILKIHEKIKNGEIDPSEILKLSEEIKK |
| | LTDTIIKIEDLEQLTRDLRR (SEQ ID NO: 68) |
| DHD27 | DRKEIVKRHQKVVELLKESSKLLRESSKLLQRLLDKTGDENLQKAVDDQDK |
| | AIKRQETAIRKSQEASKKLD (SEQ ID NO: 69) |
| DHD28 | DNSEEIKKVAKTSREVAEYSERVAKENDKVVKTLEEGKIDESELLRLLEES |
| | IKIFDTALKLHEEAYKLHQDLVRKVS (SEQ ID NO: 70) |
| DHD29 | DESEAASVAIESVQILVESVKLLEESVRILLDAVKKNGVEDLLRVAQRWEK |
| | LVDEWLKVVKRWLDNVRDIQR (SEQ ID NO: /1) |
| DHD30 | GSDKAEEVEKSVRKIEESIKKIRKSIKKAEDAVQLLKEGKIDAKDELRIVR |
| | EDLEVVKEDVEIVKEDVENVREFSS (SEQ ID NO: 72) |
| DHD31 | SDKEVSDKLLKASKKLLKVSEELLEVVRRLLKALKDDELIKKIADLLRKII |
| | DKDKKFIRTSEEIVKESR (SEQ ID NO: 73) |
| DHD32 | GSDLKEVLKTVEEAVKEIIKSSEELLQISRKILEISRVGVDEHEYISAIRE |
| | YLKALEKHIQILKKFIEILKELIRAVS (SEQ ID NO: 74) |
| DHD33 | SKEEIDKIVKKHKKKIEEHKKKVDELKKLVEEHDKRVSQDKDDKVKKLSEE |
| | VKKIIKRLEEVSKRLEEVSKKLLKVISDKR (SEQ ID NO: 75) |
| DHD34 | GSNDEELKKILETLDRILKKLDKILTRLIEVLKKSEDPNLDDKDYTELVKQ |
| | FIELIKKYEEVVKEYEEVVRQLIRLES (SEQ ID NO: 76) |
| DHD35 | SKEEIDKIVKKHKKKIEELKKLVDELKKLVEEHDKRVSQDKDDKVKKLSEE |
| | VKKIIKRVEEVAKRLEEVSKKLLKVISDKR (SEQ ID NO: 77) |
| DHD36 | GSNDEELKKILETLDRILKKLEKILTRLIEVLKKSEDPNLDDKDYTELVKQ |
| | FIELIKKFEEVIKEYEEVVRQLIRLFS (SEQ ID NO: 78) |
| DHD37 | SKEEIDKIVKKHKKKIEEHKKKVDEHKKLVEEHDKRVSQDKDDKVKKLSEE |
| | LKKISKRLEEVSKRLEEVSKKLLKVISDKR (SEQ ID NO: 79) |
| DHD38 | GSNDEELKKILETLDRILKKLDKILTRLDEVLKKSEDPNLDDKDYTELVKQ |
| | YIELVKKYEEVVKEYEEVVRQLIRLES (SEQ ID NO: 80) |
| DHD39 | DHSRKLKEILDRLRKHVKRLKEHLDELRDLVRQVPEDKLLEHVVKLSDKIL |
| | QISERAVREFTKSVDKDS (SEQ ID NO: 81) |
| DHD40 | GSDKKDELERILDEIRRLIERLDEILSRLNKLLELLKHGVPNAKEVVKDYI |
| | RLLKEYLELVKEFLKLVKRHADLVS (SEQ ID NO: 82) |
| DHD41 | DSDEHLKKLKTFLENLRRHLDRLDKHIKQLRDILSENPEDERVKDVIDLSE |
| | RSVRIVKIVIKIFEDSVRKKE (SEQ ID NO: 83) |
| DHD42 | GSDDKELDKLLDTLEKILQTATKIIDDANKLLEKLRRSERKDPKVVETYVE |
| | LLKRHEKAVKELLEIAKTHAKKVE (SEQ ID NO: 84) |
| DHD43 | MDEEDHLKKLKTHLEKLERHLKLLEDHAKKLEDILKERPEDSAVKESIDEL |
| | RRSIELVRESIEIFRQSVEEEE (SEQ ID NO: 85) |
| DHD44 | GDVKELTKILDTLTKILETATKVIKDATKLLEEHRKSDKPDPRLIETHKKL |
| | VEEHETLVRQHKELAEEHLKRTR (SEQ ID NO: 86) |
| DHD45 | DSDEHLKKLKTFLENLRRHLDRLDKLLKELRDILSENPEDERVKDVIDELE |
| | RVIRIVKIVIKIFEDSVRKKE (SEQ ID NO: 87) |
| DHD46 | GSDDKELDKLLDILEKILQTATKIIDDINKVLEKLRRSERKDPKVIETVVE |
| | LLKRHEKAVKELLEIAKTHAKKVE (SEQ ID NO: 88) |
| DHD47 | DSDEHLKKLKTFLENLRRLEDLLDKHIKQLRDILSENPEDERVKDVIDLSE |
| | RVVRTVKIVIKIFEDSVRKKE (SEQ ID NO: 89) |
| DHD48 | GSDDKELDKLLDTLEKILQTATKVVDDANKLLEKLRRSERKDPKVVETYVE |
| | LLKRLEKLIKELLEIAKTHAKKVE (SEQ ID NO: 90) |
| DHD49 | DSDEHLKKLKTFLENLRRHLDRLDKHIKQLRDILSEN (SEQ ID |
| | NO: 91) |
| DHD50 | EDERVKDVIDLSERSVRIVKTVIKIFEDSVRKLEKTKPDSKTAKELDKLLD |
| | TLEKILQTATKIIDDANKLLEKLRRSERKDPKVVETYVELLKRHEKAVKEL |
| | LEIAKTHAKKVE (SEQ ID NO: 92) |
| DHD51 | DSDEHLYKLKTFLENLRRHLDRLDKHIKQLRDILSENPEDERVKDAIDLSE |
| | RSVRIVKIVIKIFEDSVRKKEKRPIDKRDDKELDKLLDTLEKILQTATKII |
| | DDANKLLEYL (SEQ ID NO: 93) |
| DHD52 | GDPKVVETYVELLKRHEKAVKELLEIAKTHAKKVE (SEQ ID NO: 94) |
| DHD53 | DSDEHLDRLDKHLKKLKTFLENLRRHIKQLRDILSENPEDERVKDVIDLSK |
| | TVIKIFEDSVRKKERSVRIVE (SEQ ID NO: 95) |
| DHD54 | GSDDKEAZKIIDDLDKLLDTLEKILQTANKLLEKLRRSERKDPKVVETYVK |
| | AVKELLEZAKTHAELLKRHEKKVE (SEQ ID NO: 96) |
| DHD55 | DSDEHIKQLRDHLDRLDKHLKKLKTFLENLRRILSENPEDERVKTVIKIFE |
| | DSVRKKERSVRIVKDVIDLSE (SEQ ID NO: 97) |
| DHD56 | GSDDKEANKLLEKATKIIDDLDKLLDTLEKILQTLRRSERKDPKAVKELLE |
| | IAKTIIAELLKRIIEKVVETYVKKVE (SEQ ID NO: 98) |
| DHD57 | DHSRKLEEILDRLRKHIVKRLLEHLRELLSLVKENPEDKDLVEVLELSLAIL |
| | RRSLEAVEAFLKSVTKKDPDDEDLRRKADEIRKEVEEIKKSLAEVEKEIYK |
| | LK (SEQ ID NO: 99) |
| DHD58 | GSSADDVLEDILKIIRELIEILDQILSLLNQLLKLLRHGVPNAKKVVEKYK |
| | EILELYLQLVSLFLKIVKTHADAVSGKIDKKAEEEIKKEEEKIKEKLRQAK |
| | DILKKLQEEIDKIR (SEQ ID NO: 100) |
| DHD59 | DRDAHLYKLLTFLEQLVRHLDRLVKHITQLRDIVKKDPEDERAVDVIRQSV |
| | RSLEIVITVLKIFVDSVSDAARSKEAEKIVRKIRKEIDEIRQKLREIDKEV |
| | KKTTS (SEQ ID NO: 101) |
| DHD60 | GSNDKVLDKILDILDRILRLATRVIDLANKLLQVKKKSTHKDPRIVETYKE |
| | LLKIHETAVRLLLELADLHRRLKSKDEEANKRVETELDRIRKKVKDIEDKV |
| | RKLEDKVRKTAS (SEQ ID NO: 102) |
| DHD61 | NDLSKEVLKKLEKSVEELLRRVQKSVKEAQKRGLLSDELVDRHLKILNQLV |
| | KRHLELLQEVIKRSDKK (SEQ ID NO: 103) |
| DHD62 | GSDEAVKRVVEKSLKILDEVIKKSLDILRELIELQIRHAKDDESVIRASKS |
| | ALKDAIEALKKSLDEIKKALKRSADEG (SEQ ID NO: 104) |
| DHD63 | SSEEVVKVHEKVVKLIKEILELLKKIIKIHETAARDPDDKDSIKKLSDEIK |
| | KIVKRIEDISDQAKRESSDAQRKQS (SEQ ID NO: 105) |
| DHD64 | DKEEESKELLKKLKEILKRSEELLEESKELLKLAKNGEIDESELADADRKL |
| | NKKHEKLVQDIQDLLREHERQDR (SEQ ID NO: 106) |
| DHD65 | DEKKKIDKIVKETEDLLQKSEKLLQQSKEAVKRIRSQVKENEIVDRLLRIS |
| | EELLKISRRLVEISRRIASTLS (SEQ ID NO: 107) |
| DHD66 | GSSKEEVERLLKENVRLIKENLELLTRNLKLITDLVRGSNGSEEKIKTLKE |
| | LLKEYRELLKRYRKLVEDYKRLVDKHD (SEQ ID NO: 108) |
| DHD67 | EIçELIKSSRRIIEESKELIKESEEVLRRIKEILDRIRNGVDNQEDLLREI |
| | LKLLTKNLKIIQRNLKLLQDNAEILKRLVS (SEQ ID NO: 109) |
| DHD68 | GSYIEDVEKKILDVSRELIKLSRTIIKISEEINKQLQQGRDTKDLVKKYDE |
| | IIKKYTRIVQHYTELIKELQKLLS (SEQ ID NO: 110) |
| DHD69 | SPTEEAIQLSQRVIELSKRVIELSKEILKLLKRVLDLLPDLDKNEEKRLDD |
| | YDKELKEYDKELKKYEKRLKDLAS (SEQ ID NO: 111) |
| DHD70 | GSEEEEILKIQKELLRIQSEILDKQKKILDTLRSNGAVTEEVRSILEKVER |
| | LSEEAKELSKEAKELTKEVSKLIS (SEQ ID NO: 112) |
| DHD71 | SPLKELNNQLLRLLRELVKVSKKIVDLSKTIIEVLKHTDLDPRLLDSLEKS |
| | QQELDKSQKELDKVVKELTKVNKKLQ (SEQ ID NO: 113) |
| DHD72 | GSPLEDLVRKYDELVKTYEKLVEEFKKAVDKYDKAVKKAPVSKEATDSLDL |
| | IRKVLELLDRNLKLIKENAKLIKELLK (SEQ ID NO: 114) |
| DHD73 | SPTRENEKVIKENEKVISDNERVLEEVVKVVETATDRKEIQDAVDEVRKSV |
| | DKLRDSVRKLEESVRTLD (SEQ ID NO: 115) |
| DHD74 | GSPIKDISKRLLEISKRLVEISDRIVELLQRIADSKDPNKDLQKEVKDVLE |
| | EYKRLVREYREVVKEYEKVVS (SEQ ID NO: 116) |
| DHD75 | DEDEHVKQLIKNADLLRKHAELLKELVKLFQEIASQIPDDRVAKKVTDVVD |
| | RIDKILKQTEKLVRRTKQILDYSR (SEQ ID NO: 117) |
| DHD76 | GSNLEELVKLLKEVLEMHERLLRIHEDLVEAHKSNASDKESERKLKKSDKD |
| | IKESLKKIKSIIDQVRYIQS (SEQ ID NO: 118) |
| DHD77 | PVEDIIEESLRLLEESLKLLNRILKLLEDSLRKLPRSEEWRQRLDEFRKKL |
| | EDWKEELERWIEDVRYKKT (SEQ ID NO: 119) |
| DHD78 | GSDEDYESREIIDEIRKLLDRSKKIVHRSQRLVERVKSTPLSEDQEDLIRR |
| | HEETINRHRELVKELEKVLEDHERHIR (SEQ ID NO: 120) |
| DHD79 | PEEDSRRVLERFVRVSREVLKVLEEFLRVSEELLREADRDRDRRLEEYERQ |
| | VDELREEERRYKEEVDKFDKEVKYYKK (SEQ ID NO: 121) |
| DHD80 | GSPEKDENRKLLDKVRKLVEKSRRLVEELRKLVDQSTKNGLIDEKALRKQQ |
| | EVLRKVEEVLEKQERVLRELEEISYRVI (SEQ ID NO: 122) |
| DHD81 | GSPERDENRKLLDKVRKLVEKSRRLVEELRKLVDQSTKN (SEQ ID |
| | NO: 123) |
| DHD82 | GSDEKALRKQQEVLRKVEEVLEKQERVLRELEEISYRVITRGEDHKAEEDS |
| | RRVLERFVRVSREVLKVLEEFLRVSEELLREADRDRDRRLEEYERQVDELR |
| | EEIRRYKEEVDKEDKEVKYYKK (SEQ ID NO: 124) |
| DHD83 | GSDRRLEEYERQVDELREEIRRYKEEVDKFDKEVKYYKK (SEQ ID |
| | NO: 125) |
| DHD84 | GSPERDENRKLLDKVRKLVEKSRRLVEELRKLVDQSTKNGLIDEKALRKQQ |
| | EVLRKVEEVLEKQERVLRELEEISYRVITRGEDHKAEEDSRRVLERFVRVS |
| | REVLKVLEEFLRVSEELLREADR (SEQ ID NO: 126) |
| DHD85 | DLSEESKKFVEKVKKLEKESRELEKQVKKIEEDSRSVENDVQKEFLELLKR |
| | LLDIQKKVVEVLREVVKVQQYVDS (SEQ ID NO: 127) |
| DHD86 | GSDSEYESRQVLRELDTVLKDSHTVLEALRQVIRDSQDVVSKSDEESRRVI |
| | DDLEKVIQDSKKVLDDIKRLIDKSKSIKS (SEQ ID NO: 128) |
| DHD87 | NEDELLKLLTENLKLLDENLKLLRENLSLLRQANNITDKNRIREIVKQSKE |
| | IVKQSREELKQSKEIVERIKYIVS (SEQ ID NO: 129) |
| DHD88 | GSSLYELTQRYEKLVQQYEELVKDYRRLVKKLEKLKRDNKPDKRLLKEIVD |
| | VIKKSVEIIDRSLKLLEESIKILEETD (SEQ ID NO: 130) |
| DHD89 | SQERSLEILKRILDVLKESLEILKESLSILRQLASRIKNPNRKIEEILKES |
| | DKIIKESDKVLKEIEEVIRYSS (SEQ ID NO: 131) |
| DHD90 | GSDIEYESKEILELIKELLKLSRELLKESRRALELVRKSRDDSIVEEVIQV |
| | HKKVLDIHKEVLKIVRKVVEVHRRVKS (SEQ ID NO: 132) |
| DHD91 | SKKDESTKLERLAEKIDEITKRIEELVKDVKRKSSEGVDKDQQQKIDEVFQ |
| | KLLDLQREILEILDRILKVQQYILD (SEQ ID NO: 133) |
| DHD92 | GSDLEYLNRRLLQLIKTLIDLNRHLLKLIDKLKKLNSREGDEEKIKEESKQ |
| | IQEQFKEIVERSKEIIKQIKEIIKRSQ (SEQ ID NO: 134) |
| DHD93 | DFERSSRRLEKVVEDLRRSSDRLREVIDELRKSADEKDEDEDLRRARKEHR |
| | DLIEELKRALEKQEEIIKHLQELVYRQL (SEQ ID NO: 135) |
| DHD94 | GSEESEEVRKVVERIKKISRELEEVVKELDRVSKEFDRHGETDEIVREHER |
| | IVEKLEEIVKKHIKIVEELAEIVYKQQ (SEQ ID NO: 136) |
| DHD95 | SDDDSVRVLDEIVKILDESVKLLKESLKLLDDFLRTKPDDHLKEVVKESKK |
| | VVEQSKKVLDRIKKIIYESK (SEQ ID NO: 137) |
| DHD96 | GSDLLYLSKELLKLVRELLKLSRELVELSRRLVNSTHKSPELVKKYDKLVK |
| | KYQDLLKKLADVADEYLRQRS (SEQ ID NO: 138) |
| DHD97 | DEKDYHRRLIEHLEDLVRRHEELIKRQKKVVEELERRGLDERLRRVVDRER |
| | RSSERWEEVIERFRQVVDKLRKSVE (SEQ ID NO: 139) |
| DHD98 | GSDAYDLDRIVKEHRRLVEEQRELVEELEKLVRRQEDHRVDKKESHEILER |
| | LERIIRRSTRILTELEKLTDEFERRTR (SEQ ID NO: 140) |
| DHD99 | DERYRAREHIRRVEEHTKRLRHILKRLREHEEKLRRELKPGDEITESVDRF |
| | KKIVDQFEESIKKFETVSEELRKSDS (SEQ ID NO: 141) |
| DHD100 | GSDRQRILDRLDKILEKLDDILKKLKDILETLSKDDVSDRRHKDLVEKFRE |
| | LVDTHHKLVERYRELVYQNR (SEQ ID NO: 142) |
| DHD101 | GSDEITESVDRFKKIVDQFEESIKKFETVSEELRKSIS (SEQ ID |
| | NO: 143) |
| DHD102 | GSDPQRAADRLDKILEKLDDILKKLKDILETLSKDDVKDRRAKDLVEKFRE |
| | LVDTHHKLVERYRELVYTATAGSDLARELIRRVEEHTKRLRHILKRLREHE |
| | EKLRR (SEQ ID NO: 144) |
| DHD103 | NADDQLASSIKKLEDSIDQLIKIVRKFEESVKKLQKHGVDQHHVEILRKIV |
| | EIFRQHIEKLKKHLEKLRYTSS (SEQ ID NO: 145) |
| DHD104 | GSDKEYLVTEHEKLVREHEKIVSEIEKLVKKHEAGVDESELEEILKKVEKL |
| | LRKLDEILEQLTQLLRKTE (SEQ ID NO: 146) |
| DHD105 | GSDQHVVEILRKIVEIFRQHIEKLKKHLEKLRYTSS (SEQ ID |
| | NO: 147) |
| DHD106 | GSDAEYLVTEHEKLVREHEKIVSEIEKLVKKHEKGVDESELEEILKKVEKL |
| | LRKLDEILEQLTQLLRKAEKHIDKHSKAADQLATSIKKLEDSIDQLIKIVR |
| | KFEESVKKLQKH (SEQ ID NO: 148) |
| DHD107 | DEDDDIRRVLDESRRVLEHSRRVLKRSEEVLEKASRKKEKDTEEIEKHLKR |
| | LREHAKKLEKHRRELDDFLYKEI (SEQ ID NO: 149) |
| DHD108 | GSRDKYLLERLNDILKKLDEIVDKLSDILKRLKDVRHDDRLQELVERYKEI |
| | VKEYKRIVEEYEKLVREFEEQQR (SEQ ID NO: 150) |
| DHD109 | DRDYEDKEFKKIIKELEDVQEELKKLQEKIKRFSSELEEPNELLKEQLKVN |
| | EEQLEVNKKILKILRDQLKQNE (SEQ ID NO: 151) |
| DHD110 | GSDAEYKVRESVKRSKESVKHSEDVVDKLNKSVKLSESGHSDAEKASRELV |
| | KLVREVVELSREVIKLSEKVLRVIS (SEQ ID NO: 152) |
| DHD111 | DLQYKQEKLIRHFDRVVREWDKLVRKFSKVLEKQKHESKDKELEEASRRVD |
| | ELIKRLREQLKRSKEILRRLKELSRKSS (SEQ ID NO: 153) |
| DHD112 | GSDWEELLRRLEKVLQEYEEIVKELIDLIERLIKVSEDKSKDASEYKKLVT |
| | ELEKLISKLEEISKKLEELVKEYEYKTE (SEQ ID NO: 154) |
| DHD113 | DAKDELEKSLQEIEESLKELKKLLEELDKSLRELTSQGRNKKLEEHIKKVQ |
| | KFIELVKKYIKAVQDYLKEVRYDNS (SEQ ID NO: 155) |
| DHD114 | GSDKERAARATEEMVKLTKKLLKAVEDLVRDVRRLLKEGLISEKHARIAET |
| | ILEVEKKHAKIIKKHVDIVKYDES (SEQ ID NO: 156) |
| DHD115 | GSPLKERLLEIQRDLDRVLEEVVERLLRIQERLDSVVERKPPDVHEEYKYI |
| | VDEIREIVERVVREYEEIVKRIDEEVR (SEQ ID NO: 157) |
| DHD116 | GSEEDERIRYDLDRRKDVRRKLEEIRQRVRELEKKLRDAGIIRRDEKELLR |
| | ELIETSKDILRLVEELLKKIIDKSEDLLRKTE (SEQ ID NO: 158) |
| DHD117 | GSDEEDYINENVEKDVRDIEDDVRRINERIRELLEKIRTEEVLQRVLEEHH |
| | ELVERVLRKLVEILRKHEEENR (SEQ ID NO: 159) |
| DHD118 | GSDEEEYYKEKLHKLLREIEELLKHYRELVRRLEELVKRGELDKDTAAHIL |
| | ERLSELLERIIRRVAHILRRLSEERR (SEQ ID NO: 160) |
| DHD119 | GSDEDEISYDSKRRVEEIVRQAREKSEKSRKDIEDVAEVLRKGDVSEKEVV |
| | DELVKVLEEQVKVLREAVERLREVLKKQVDDVR (SEQ ID NO: 161) |
| DHD120 | GSDIVELVDHLLKRSLKLLEELAELVRRLLEKSTELLKRRTEEHKEEVVEE |
| | SEYMVRELEERLRRVVDESEKLVRDADKHIR (SEQ ID NO: 162) |
| DHD121 | GSKEKDIVKTLVDLLRENLETLERLIEEVVRLLKENVDVRDEGRDDKDSER |
| | ILRDIKRRIDEAAKESREIIERIEKEVEYRSR (SEQ ID NO: 163) |
| DHD122 | GSPEVDVLRRIVREILKASEELLRLLRKLIDEALKLSERKRDSQEYREVVD |
| | RVKKELERLLDEYRKLVEELKEKLRYDTR (SEQ ID NO: 164) |
| DHD123 | GSDKRYESEKLKRRLDEAVEKVREVVERVERESDRVLEEVRRRRESKEVVD |
| | KVIEDNDKALEDVLRVVDEVAKVVRDVVRENTR (SEQ ID NO: 165) |
| DHD124 | GSPREYHSKDILRKVDEILERIRRHADRVKKKSERLKRENVDVNEHSKDVK |
| | RVIRELLELVKELLRLAKKHSDDQQE (SEQ ID NO: 166) |
| DHD125 | GSDEDEILYHSERLLQKLKKELDDLKEKSRELLEELKKEDPDDRLIERIIR |
| | LHDEVLKDLDEVLKNILEVHREVLERLR (SEQ ID NO: 167) |
| DHD126 | DKLDRLLKIHEEALRRAEELIKRLLDIHRRALDLARRGELDDYLLKESERE |
| | LREIIRRAREELKESRDRLEEISR (SEQ ID NO: 168) |
| DHD127 | GSPKEELERRVLEEVRLNEKLLEI IRRAAELVKRANDELPETEKLREIDR |
| | ELEKKLKEIEDELRRIDKELDDALYEIED (SEQ ID NO: 169) |
| DHD128 | GSPKLDKLRELLERNLEKLREILEEVLKILRINLERVREDIRDEDVLQEYE |
| | RLIRKAEEDLRRVLKEYDDLLKKLVYELR (SEQ ID NO: 170) |
| DHD129 | GSKEDESVKRAEEIVRILLKLLEDSLREAERSLRDIKNGEDEHNLRRISEK |
| | LEELSKRETETIERLLRELQYTSR (SEQ ID NO: 171) |
| DHD130 | GSPNQELLDRVRKILEDLLRLNEELVRLNKELLKRALEMRRKNRDSEEVLE |
| | RLAEEYRKRLEEYRRELEKLLEELEETIYRYKR (SEQ ID NO: 172) |
| DHD131 | GSDESEEAQHEVEKVLDDIRRLSEHLQKRLEEVLEEVYELRREGSDRTEVV |
| | ELLKEVIREIVRVNREALERLLRVVEEAVKRNE (SEQ ID NO: 173) |
| DHD132 | GSDEEELVETVKRIQKEILDRLTELAKLLVEIQREIKKLKDEGEDDKELKR |
| | LSDELEEKVRQVVEEIKRLSDELEETVEYVSR (SEQ ID NO: 174) |
| DHD133 | GSDEEEEVVRRAEELVKEHEELIERVIRTHEELVYKLEDQGADKKLVDVLK |
| | RVVEESERVAREIVKVSRELIRLLEEASR (SEQ ID NO: 175) |
| DHD134 | GSSKEEILKELEDLQRRLIEELKKLQERVVELLEELIKRLRDRGRDDKHLK |
| | RLVKEVRRLSEEVLRSIKEVSDRVRYQLR (SEQ ID NO: 176) |
| DHD135 | GSDKEEESEYLLRDLVRLLEKVKEKIEEVNREVEKLLKKVKDGRLDRREVL |
| | REILRLNRELAEIIKEVVDRIRHVVERSER (SEQ ID NO: 177) |
| DHD136 | GSDLHEVVYETKELLKRIEEVVEELRKKSEDIIRKAERGEISEDELKRLQE |
| | EIAREAKKLLDEIKRVLERHLEQTL (SEQ ID NO: 178) |
| DHD137 | GSPVEEIIKEVVKRVIEVQEKVLRIISHAVKRVVEVQKKYDPGSEESNRVV |
| | EEVKKTIEDAIRESDEVVDEVVKRIQYTVR (SEQ ID NO: 179) |
| DHD138 | GSPEQEIADRILTEIRESQKELERLARKILKLLDESQEKAKRGRLSEEESD |
| | ELLERIKKELDELLERSKELLKKIEYELR (SEQ ID NO: 180) |
| DHD139 | GSDEDKEANRVLDEVLKTVRDLLETANEVLKEVLYRLKRTDDQEKVVRTLT |
| | EVLKEHLKLVEEIVRILDKVLKEHLETEK (SEQ ID NO: 181) |
| DHD140 | GSPEDDVLRRLEEVSEKILRVAEDVARQLREVSEKITQGKVDRKEWEEDIK |
| | RLKRELEELLREWKEEIERLTYELR (SEQ ID NO: 182) |
| DHD141 | GSRREEVVKRIRELLKRNKELIDRIRELLEENEYLDKDARDKDVLRRSVEL |
| | LEELVRILEESVELAKEIIKLLREVVE (SEQ ID NO: 183) |
| DHD142 | GSDEKEDNRRLQHKIERILEKNEDLQRKLEEILELLERGEADEEKIDRLRK |
| | AVEDYRRVVEEIKEDVKRHKYTVR (SEQ ID NO: 184) |
| DHD143 | GSDEKEEAKKASEESVRTVERILEELLKASEESVELLRRGEDAKDVVERSK |
| | EALKRVKELLDEVVKRSDEILKYIHN (SEQ ID NO: 185) |
| DHD144 | GSDEKKLINEVVETQKRLIKEAAKRLSEVVRHQTELIRELREKNVDDKDVE |
| | KLLKESLDLAEEIVRRIKELLDESKKLVEYVSN (SEQ ID NO: 186) |
| DHD145 | GSPDMDEVKRVLDELIEIQEEILREIKRVLEKLIKIQEDNGSEYESREVVR |
| | EIVEIARKLVERSRRVVKKITETLQ (SEQ ID NO: 187) |
| DHD146 | GSDERYATREIVERIERIAREILKRTEEIVREVREVLSRDVDQEEVVRRLA |
| | DLLRESVELVQHLVRRVEELLQESVERKK (SEQ ID NO: 188) |
| DHD147 | GSPEREALREVLEDLKRVTDRLRELVERVLEELKKVTDHVDSERILRESRR |
| | VLKELKDIEEILRESEKVLEKLKYTED (SEQ ID NO: 189) |
| DHD148 | GSPAREILEEVVKKHLEVVEDAARILEEIIREHEKAVREDRDKKELEEISR |
| | DLLRKAREALKKVKDISDDLSREIEYVAS (SEQ ID NO: 190) |
| DHD149 | GSPVEEAIKKVIDDLRDVQRKIRELVEELIRLLEEVQRDNDKRESEYVVER |
| | VEEILRRITETSREVVRKAVEDLS (SEQ ID NO: 191) |
| DHD150 | GSDSDEKAEYLLKEMERVVRESDEVVKKILRDLEEVLERLRRGEISEDDVT |
| | EILKELAERHIRAIEELVRRLRELLERHKR (SEQ ID NO: 192) |
| DHD151 | GSPVEEVLKELSEVNERVRDIAREIIERLSEVNEEVKETDDEDELKKISKK |
| | VVDEVEDLLRKILEVSEEVVRRVEYHDR (SEQ ID NO: 193) |
| DHD152 | GSPKEDILREVLRRHKEIVREIVRLVREAVETHLELVKRNSDDRDAQDVIR |
| | KLEEDLERLVRHAQEVIEEIFYRLH (SEQ ID NO: 194) |
| DHD153 | GSPRSYLLKELADLSQHLVRLLERLVRESERVVEVLERGEVDEEELKRLED |
| | LHRELEKAVREVRETHREIRERSR (SEQ ID NO: 195) |
| DHD154 | GSDREYIIKDILDSQEHLLRLIEELLETQKELLEILKRRPDSVERVRELVR |
| | RSKEIADEIRRQSDRNVRLLEEVSK (SEQ ID NO: 196) |
| DHD155 | GSDEKDERHVIESVERLIEDIKRLLKTLRELAHDDSDKKTVKEVLDRVKE |
| | MIERHRRELEEHRKELERAEYEVR (SEQ ID NO: 197) |
| DHD156 | GSESEDRIKELLKRHIELVERHEELLHEIKKLIDLEEKDDKDREEAVKRID |
| | DAIKESEEMLEESKEILEEIEYLNR (SEQ ID NO: 198) |
| DHD157 | GSSLEDSVRLNDEVVKVVERVVRLNQEVVRLIKHATDVEDEETVKYVLERV |
| | REVLDESREVLKRVHELLEESERRLE (SEQ ID NO: 199) |
| DHD158 | GSHEKDIVYKVEDLVRKSDRIAERAREIVKRSRDIMREIRKDKDNKKLSDD |
| | LLKVTRDLQRVVDELEELSRELLRVAEESRK (SEQ ID NO: 200) |
| DHD159 | GSPELDEVKKLIDELKKSVERLEESIREVKESIKKLRKGDIDAEENIKLLK |
| | ENIKIVRENIKIIKEIIDVVQYVLR (SEQ ID NO: 201) |
| DHD160 | GSDEEEIEELLRELEKLLKKSEEALEESKKLIDESEELLRRDRLDKEKHVR |
| | ASEEHVKLSEEHLRISREIVKILEKAVYSTR (SEQ ID NO: 202) |
| DHD161 | GSDESDRERKIVEESDEIVKESRKLAERARELIKESEDKRVSEERNERLLE |
| | ELLRILDENAELLKRNLELLKEVLYRTR (SEQ ID NO: 203) |
| DHD162 | GSDEDDELERLLREYHRVLREYEKLLEELRRLYEEYKRGEVSEEESDRILR |
| | EIKEILDKSERLWDLSEEVWRTLLYQAE (SEQ ID NO: 204) |
| DHD163 | GSDKKDASRRAIRVLHEFVRVSEEVLEVLRKSVESLKRLDVDEKIKRTHDR |
| | IEEELRRWKRELEELIERLREWEYHQD (SEQ ID NO: 205) |
| DHD164 | GSDDEEEDKRLLEEVKRSLDTDERILEKLRHSLERQLEDVDKDEDSRRVLR |
| | ELDEITKRSREVVKRLRKLAYESK (SEQ ID NO: 206) |
| DHD165 | GSDKEYKLDRILRRLDELIKQLSRILEEIERLVDELEREPLDDKEVQDVIE |
| | RIVELIDEHLELLKEYIKLLEEYIKTTK (SEQ ID NO: 207) |
| DHD166 | GSPSKEYQEKSAERQKELLHEYEKLVRHLRELVEKLQRRELDKEEVLRRLV |
| | EILERLKDLHKKIEDAHRKNEEAHKENK (SEQ ID NO: 208) |
| DHD167 | GSRDRKISEELIKALEDHIRMLEELIRAIEEHIKLAERGVDEKELRESLEE |
| | LKKIVDELEKSLEELRKLAERYKYETR (SEQ ID NO: 209) |
| DHD168 | GSPKEESVEELKRVIDKHEEILRELKRVLEEHERVSHDEDENELRRSLERL |
| | KHILDRLHESLKELHELLKKNEYTER (SEQ ID NO: 210) |
| DHD169 | GSDHEYWVKIVERILRVMEKHAEIVKKHLEIVERVVREGPSEDLRRKLKES |
| | LREIEESLRELKELLDELDELSEKTR (SEQ ID NO: 211) |
| DHD170 | GSDEEYVERSQRRLKRLLEEYIKVVEEHARLVERNERDDKELKRSIDELDK |
| | LTKELLELVKRYKELVDKTET (SEQ ID NO: 212) |
| DHD171 | GSDKEEIVKLQDEVIKTLERHLDILRKHIDLLEKLKDHLSEELKERVDRSI |
| | KKLEESIKRLERIIEELQELAEYSL (SEQ ID NO: 213) |
| DHD172 | GSREEELKESAEELERSVRELKKEADKYKEEVDRLHYRGKVDKDWVRVVEK |
| | LIKLVEEHLELIREHLELLKEERR (SEQ ID NO: 214) |
| DHD173 | GSDMEYELKKSAEELRKSLEELKRILDELHKSLRELRRHGDDEEYVQTVEE |
| | LRKELEEHAKKLEEHLKELERVAT (SEQ ID NO: 215) |
| DHD174 | PEYELKKSVDDLKRDVDRLVEEVEEVFELSKERLREDRKHLELVEEMVRLI |
| | EKHLELIKEHLKLADDHVR (SEQ ID NO: 216) |
| DHD175 | GSREKDESKELNDEYKKLLEEYERLLRRSEELVKRAKGPRDEKELKRILEE |
| | NEDILRREKEILERTKEISEEQKYRRR (SEQ ID NO: 217) |
| DHD176 | GSDKDERQERLNEESDKSNEESERSNRESEELNRRARGPNDEKELQEILDR |
| | HLELLERNQRLLDENKEILRESQYLND (SEQ ID NO: 218) |
| DHD177 | GSENKYILKEILKLLRENLKLLHDILRLLDENLEELEKHGAKDLDDYRRKI |
| | EEIRKKVEDYREKIEEIEKKVERDR (SEQ ID NO: 219) |
| DHD178 | GSESEYTQEEILELLKESIKLLREILRLLEESEELWRRENTKSERSEEIKE |
| | RAKEAIKRSEEILERVKRLSDHSR (SEQ ID NO: 220) |
| DHD179 | GSDEEEANYVSDKAVKIAEDVQELLKELLELSEVVRRGEVDEDEYDRVLRK |
| | LQEVMKEYEEVLKEYEEVSRKHE (SEQ ID NO: 221) |
| DHD180 | GSPEKYLIKIQEELLRRHAEILEDLIRKVERQVDLRRKVDERDEDLKRELE |
| | RSLRELERLVRESSRLVEEIRELSKEIKR (SEQ ID NO: 222) |
| DHD181 | GSDEEYELERISRESKELLERYKRLLREYQELLKELRHVKDLDRAVKITHE |
| | LMRVSKELVEISHRLLELHERLVRRRK (SEQ ID NO: 223) |
| DHD182 | GSEKEYIEKLSRKIEEDIRRSEERAKDSERLVRRLEELAKRKRLDLDDVLR |
| | VAEENLEILEDNLRILEEILKEQDKSNR (SEQ ID NO: 224) |
| DHD183 | GSPHEEVVELHERVMEISERAVELIQRIIDIIRRIREDDKDIEKLVKTIRD |
| | LVREYEELHRELEEIDEEIYKKSE (SEQ ID NO: 225) |
| DHD184 | GSDHEDVVRLHEDLVRKQEDARRVLEEIVRLAEEIVEVIKKDEKDKDRVTR |
| | LVEEIEKLVEEYKKKVDEMRKISDEIKYRSR (SEQ ID NO: 226) |
| DHD185 | GSRAREVVKRAKRIIEEWQKILEEWRRILEEWRRLLEDERVDDRDNERIIR |
| | ENERVIRENEKIIRDVIRLLEELLYERR (SEQ ID NO: 227) |
| DHD186 | GSREDEELEEEIDRIRQMVEEYEELVKEYEELTEKYKQGKVDKEESKKIIE |
| | KSERLLDLSQDAVRKVKEIIRRILYTNR (SEQ ID NO: 228) |
| DHD187 | GSPKEEIVKLHDESAELHRRSVEVADEILKMHERSKDVDDERESRELSKEI |
| | ERLIREVEEVSKRIKRLSEEVEYLVR (SEQ ID NO: 229) |
| DHD188 | GSPLEEILKIQRRINKIQDDINKILHEILRMQEKLNRSSDKDEVEESLRRI |
| | RELIKRIKDLSKEIEDLSREVKYRTT (SEQ ID NO: 230) |
| DHD189 | GSPEDEHVYVVREIYEVLREHAEVLEENREVIERLLEAKKRGDKSEELVKE |
| | LKKSIDKLKEISRKLEEIVKELEKVSEKLK (SEQ ID NO: 231) |
| DHD190 | GSDEDETSYRILELLREIVRASRELIRLSEELLEVARRDDKDETVLETLIR |
| | EYKELLDRYRRLIEELTRLVEEYEERSR (SEQ ID NO: 232) |
| DHD191 | GSTQEEINRIQHEVLRIQEEIDEILRDIVEKLKAISRGELDHEVVKDVEDK |
| | VREALEKSEELLDKSRKVEYKSE (SEQ ID NO: 233) |
| DHD192 | GSDEEELNRELLEKSRLVDINRDIIRTAQELIEMLKD SKDGRVDEDTKRE |
| | LRDKLRKLEEKLERVREELRKYEELLRYVQR (SEQ ID NO: 234) |
| DHD193 | GSDEKDRVYEILKEVQRLVKEYRDISKEIEDLVKHYEHITDDEAQEVSKEL |
| | IDKSLRASEIVRELIRLIKELLDELE (SEQ ID NO: 235) |
| DHD194 | GSDEEDVLYHLRELLEELKRVSDDYERLVREIKETSERKDRDTKENKDMLD |
| | ELVKAHREQEKLLERLVRLLEELFERKR (SEQ ID NO: 236) |
| DHD195 | PREQAIRESEEIIRISKKIIEILERTRSSTAREAMKWAKDSIRLAEESKYL |
| | LDK (SEQ ID NO: 237) |
| DHD196 | IEDDVKKIQDSTKKAQKETIEALERSTSSTARKQMEEQKEQIRLQKEAMYL |
| | LKK (SEQ ID NO: 238) |
| DHD197 | SREEIAKLQEEVIKLQRRVIELQKEVIELQRRAKELTSSYTKEILEIQRRI |
| | EEIQREIEEIQKRIEEIQEEIQRRT (SEQ ID NO: 239) |
| DHD198 | SDEEIKRLSEEVIQLSRRVIKMSREAIKLSREVQKLTPSYQKRIKEIADRS |
| | IELARESTEIAKRSEKIAEESQRRT (SEQ ID NO: 240) |
| DHD199 | PAKDEALKMANESLELAKKSARLIQESSSKEILERIEKIQRRIAELQDRIA |
| | YLIKK (SEQ ID NO: 241) |
| DHD200 | PAKDEALRMIDESRELIKKSNELIQRSSSKEILERILEIQRKIAELQKRIQ |
| | YLLKS (SEQ ID NO: 242) |
| DHD201 | TDEARYRSERIVKEAKRLLDEARRRSEKIVREAKQRSNSEDAKRIMEENLR |
| | ESEEAARRLREIIRRNLEESRETG (SEQ ID NO: 243) |
| DHD202 | TREALEYQRKMAEEIEDLLREALRRQEEMVREAKQRSLSEEFKRIMERILE |
| | EQERVMRLAKEALERILEEQKRTG (SEQ ID NO: 244) |
| DHD203 | SERTKREAKRSQEEILREAKEAMRRAKESQDHRQNRDGSNSEDLERLSQEQ |
| | KRELEEVERRLKELAREQKYKLEDS (SEQ ID NO: 245) |
| DHD204 | SEDLKRILKEITERELKLMQDLMEILKKITEDENNLDSNNSEDLKRSIEKA |
| | RRILDEALRKLEESARRAKYIQEDN (SEQ ID NO: 246) |
| DHD205 | TEDEIRESLKWLDEVLQELREIARESNEVLERNRQKSRSDKLREDIERYKK |
| | RMEEARKKLDDQLNKYKKRMDENRS (SEQ ID NO: 247) |
| DHD206 | TEEELKESKKFAEDLARSARRALKESKRVLEEISQASRSKKLEEIVRRYKE |
| | QVKRWQDEWDERAREYRKRMKENRS (SEQ ID NO: 248) |
| DHD207 | TKTEEIERLAREIKKLSEKVERLAQEIEELSRRVKEENSTDRELKEANREI |
| | ERAIREIEKANKRMEEALRRMKYNG (SEQ ID NO: 249) |
| DHD208 | TKTEEHERLAREISKLADEHRKLAKIIEELARRIKEENLTDDELREAIRKI |
| | EDALRKNKEALKIMKEAAERNRYNT (SEQ ID NO: 250) |
| DHD209 | TKKEESRELARESEELARESEKLARKSLELARRAESSGSEEEKRRIIDENR |
| | KIIERNREIIERNKEIIEYNKELIS (SEQ ID NO: 251) |
| DHD210 | TKDEESLELNRESEELNRKSEELNRKSKELNDRAESSNSEEEEKEILREHK |
| | EILREHLEILRRHKEILRRHKYLTS (SEQ ID NO: 252) |
| DHD211 | TREELLRENIELAKEHIEIMREILELLQKMEELLERQSSEDILEELRKIIE |
| | RIRELLDRSRKIHERSEEIAYKEE (SEQ ID NO: 253) |
| DHD212 | SEDIAREEKELLRRLKEIIERNQRIAKEHEYIARERKKLDPSNEKERKLLE |
| | RSRRLQEESKRLLDEMAEIMRRIKKLLD (SEQ ID NO: 254) |
| DHD213 | DRQKLIEENIKLLDKHIKILEEILRLLKKDIDLLKKSSSEEVLEELKKIHR |
| | RIDKLLDESKKIHKRSSEIVKKRS (SEQ ID NO: 255) |
| DHD214 | DECKLIESSQRLQEKSERLLEKFEQILREASDLYRKPDSEELLRRVEKLLR |
| | ELEKLIRENQDLARKHEKILRDQS (SEQ ID NO: 256) |
| DHD215 | DRCELIRENIELLKKHIKIVKEIQKLIETFIELLKKSSSEEILRRLKKILK |
| | RIEKLYRESQEIHKRSEEIAKKRQ (SEQ ID NO: 257) |
| DHD216 | DEERLIDKSRELQKESEELLKELLKIFKRIEELLEKPDSEELIREIKKLLE |
| | TLSEIHKRNEKLARTHEEILRQQS (SEQ ID NO: 258) |
| DHD217 | STRDVQREIAKAFKKMADVQKKLAEEIKRHVKNVEKKNKDNDEYRKIATEL |
| | LKKATESQKKLKELLDRIRKSDS (SEQ ID NO: 259) |
| DHD218 | DKDDRSTSLLKRVEKLIDESDRIIDKFTTLIELSRNGKIDDDQYKKELKEI |
| | LELLKKYDKHVKEVEELLKRLNS (SEQ ID NO: 260) |
| DHD219 | SKRKALEVSERVVRISEKVVRVLDESSDLLKKSYDDSDKFAELIDRHEEKI |
| | KKWKKLIKEWLEIIQRHKS (SEQ ID NO: 261) |
| DHD220 | SAEEFVKLSEEAVKRSKEILDIVRKQVKLVKAGVDKHEITDSLRKSEKLIE |
| | EHKELIKTHRDLLRREN (SEQ ID NO: 262) |
| DHD221 | SSTEILKRFKRALRESEKIVKHSRRVLKIIREVLKQKPTQAVHDLVRIIET |
| | QVKALEEQLKVLKRIVEALERQS (SEQ ID NO: 263) |
| DHD222 | DKQKEIKDILEKTRRIAEESRKIAEKFDEIIKRSTEGKIDESLTKELEELV |
| | KEVIKLSEDDARTSDDLVRKES (SEQ ID NO: 264) |
| DHD223 | DEDESIKLTRKSIEETRKSLKIIKEVVELIREVLKHIKDLDKEIFERIDKI |
| | LDKYKKQVDTYDEILKEYEKKQR (SEQ ID NO: 265) |
| DHD224 | SELDEQKELIKKQEKLIEEQQRLLSKIRRMFKERVKDQELLREIQKVLKRS |
| | QEIVETSKKILDRSDKITE (SEQ ID NO: 266) |
| DHD225 | DQKEINTRIVEKLERIFKKSKEIVRQSERVISTIEKKTEDERELDLLRRHV |
| | KIVREHLKLLEELLKIIKEVQKESE (SEQ ID NO: 267) |
| DHD226 | DTEELVKRLNELLKELSKLVKEFIKILETYRKDQTKDTSKISERVDRILKT |
| | YEDLLQKYKEILEKIEKQLS (SEQ ID NO: 268) |
| DHD227 | DYARLIDQAVEVTRKVVEVNVTVARVNDKFAKHLGDEELRRVSEHLKEVSK |
| | DLQEVAKKSKDAARQVK (SEQ ID NO: 269) |
| DHD228 | DVSKVAEEYLQISKTLVDISRTLLEISERLVRLVRTVADDRSEVKKAIEDS |
| | IEVLKTSEEVVRQIKRASDKLVKAIS (SEQ ID NO: 270) |
| DHD229 | DAKEIQRRVVEIQTEVVKLQKKAVDIIRKIIEAFNNSNIDQSLLEAAKEIV |
| | KEIDKLEKLTESLLEESKKLLKRSS (SEQ ID NO: 271) |
| DHD230 | SAEEVVKLAKIFLELLRESIKLLKRSVDLLRKSSDPSLDKSEAEKVSREIE |
| | KVSDTSLKLSKKALDVVKRALKVAS (SEQ ID NO: 272) |
| DHD231 | DEKDAARKARKVSEEAKEASKKIEKALEESKRILNTLKQKKDEQEVKVIKE |
| | HEDVLRQIEKIQKQVLEIQKEVAKLLESLD (SEQ ID NO: 273) |
| DHD232 | SADDVARASEKVLRVARESAKAADKSLEVFKEVVKRGDKEAFLQVVKINEE |
| | VVKINITVIRILIEVSKTAT (SEQ ID NO: 2/4) |
| DHD233 | DEYVKETLKQLREALASLREADKRITELVKEARKKPLSEAARKFAEAIVTH |
| | VKVVVEHVEVVLRHVEVLVEAKKNGVIDKSILDNALRIIENVIRLLSNVIR |
| | VVDEVLQDLD (SEQ ID NO: 275) |
| DHD234 | DASDVIRRIHELFEEVHRLIEAVHRAIEDVAKAAQKKGLDESAVEILAELS |
| | KELAKLSRRLAEISREIQKVVTDPDDKEAVERLKEIIKEIKKQLDELRDRL |
| | RKLQDLLYKLK (SEQ ID NO: 276) |
| DHD235 | SEDKAHHDIVRVLEELIKIHDELMKISEEILKATSDSTATDETKEELKRRS |
| | KEAQKKSDTLVKIVKELEKESRKAQS (SEQ ID NO: 277) |
| DHD236 | DDEEKYRQIIREAQEISKTAKRILRDAQEISKRIRHQGVDRSEHQRLVDLL |
| | RELIKEHHKLLRRQQEADTRND (SEQ ID NO: 278) |
| DHD237 | DRKDKARKASEKLEEVIQRWKTVADKWKKMVDLVSNGKLSQEEVARVTEEL |
| | LKIQTELAKLLEEHAKVLQESAS (SEQ ID NO: 279) |
| DHD238 | SDEESIKTQSELIKTSEELLKDVKRIDEELQKLRDDPTLDESELKKRVKEW |
| | SDRVRKAKEISRKIQEIVKESKKRSS (SEQ ID NO: 280) |
| DHD239 | DKDEELRKVIEKYREMVKEYRKVIREYEEVIKSSKTIDKSSLISLSRKMVE |
| | LSQRVIDVSDEVAKVLSRKQS (SEQ ID NO: 281) |
| DHD240 | TDEERLKKQTKELKEQTKQLEKQKDLLEKISNGEISKDEIQEIIKESKKIA |
| | KESQKALDSSRKALEEVS (SEQ ID NO: 282) |
| DHD241 | DEKEVSKEIIKVLKDIAKVQQKVIEVSQRLASVLRADDDNVVKRALEEYEK |
| | ILEELRELNKEIEKLTDKYRKVTS (SEQ ID NO: 283) |
| DHD242 | DSDEQTKELEKLTELAKRHVEKLKKQTKESREVDSNKLWKSKDVKDKLSES |
| | EKELQKLSDQDKKAKDALESSRRKND (SEQ ID NO: 284) |
| DHD243 | DAEEQLKLLTKLLRHQQRLLQLIKESLKLIEKIDQSSQENQDEIRKWREVT |
| | KKLRELIKTSEKLVRELEKSYKKSS (SEQ ID NO: 285) |
| DHD214 | SLRDVVRRYQELVRRYDELIKTLTEILKKYQKKGAEDKDASTELVKAVRTS |
| | LKLSKELLKLNSELLKEDS (SEQ ID NO: 286) |
| DHD245 | SKEELKRKLDELKKRSDTLKELSKKLKEISERNPDDKSVHRTIIRIHREFV |
| | KNHKEIVRVIEEIVSDKS (SEQ ID NO: 287) |
| DHD2/6 | SKQDEHDRLLKIHDKLVKQHDELLKLLTKLSRAGDSVTKKKLEEILRKLQE |
| | VSKQLEESLKDADKVSKDIN (SEQ ID NO: 288) |
| DHD247 | TVQSLLEQHVKIVKRSIEILERHTQILQDIARSQGVSKELEDVERQVKEYR |
| | KEVKKLEEDLRQLSRNSK (SEQ ID NO: 289) |
| DHD2/8 | SDSDRIEKLIRESTELLKEQQKLAKRSRELAETVESLPLTEEYLKQQREHQ |
| | KKIEKLLKDSEKHLEELKRLVKSEK (SEQ ID NO: 290) |
| DHD249 | DSEKRIEDILRIDLELAKRDAELVKEHIKLVKRIDLSEELKKQVEDVEKES |
| | KKLEDSSEKLVQKVRKRSS (SEQ ID NO: 291) |
| DHD250 | DEEERAKDLRKYLEEQTQYYRTVTEHLRNLEKVVEELERRGKPSSELQQIL |
| | ERSQRIYKETTEIYDTSKKLIEELDKHHR (SEQ ID NO: 292) |
| DHD251 | PLEDILKRHLDKVRELVRLSEEVNKLAKEVLDILKDKRVDEKELDKVLKEL |
| | EKVVEEYERAVKESRDLLRELRETTR (SEQ ID NO: 293) |
| DHD252 | DKERLLEIHERIQKLLDRNLEIIERLLRLLREARDIKDDDKLDKVIKRLKE |
| | LSEESKDILDKIKELLKESEKELT (SEQ ID NO: 294) |
| DHD253 | PEDEVIRVIEELLRIAAEVDEVHRRNVEVQEEASRVTDRERLERLNRESEE |
| | LIKRSRELIEEQRKLIERLERLAT (SEQ ID NO: 295) |
| DHD254 | DLEELIKEYAEVVRRHHKAVRDLERLVRELANAKHASEEELKRIATEILRI |
| | VKELIRVQERLIKLSEDSNEESR (SEQ ID NO: 296) |
| DHD255 | PTDEVIEVLKELLRIHRENLRVNEEIVEVNERASRVTDREELERLLRRSNE |
| | LIKRSRELNEESKKLIEKLERLAT (SEQ ID NO: 297) |
| DHD256 | DNEEIIKEARRVVEEYKKAVDRLEELVRRAENAKHASEKELKDIVREILRI |
| | SKELNKVSERLIELWERSQERAR (SEQ ID NO: 298) |
| DHD257 | PKEDIDRVSRELVRVHKELLEVLRKSTEIVEAVARNEKDERTIEEVLEEQE |
| | RAVRKLEEVSKKHKEAVKRLK (SEQ ID NO: 299) |
| DHD258 | ELERLSEEIQKLSDRLIELIRRHSKVLEEIVRLLKHKDNDEREVRRLLKLL |
| | RDLTRRYEEVLRKVEEIVKRQEDESR (SEQ ID NO: 300) |
| DHD259 | PEEDILRLLRKLVEVDKELLEVVRESTEVVRLVARNEKDVETVERVLRKQE |
| | EVVRKYERVSRELEEAVRRLK (SEQ ID NO: 301) |
| DHD260 | ELKDLVEEIVKLSKENLKLWEDHSRVLEEIVRLLKHKDNDEREVRRLLKLL |
| | EDLTRRAEETSRRIEEIVKEAEDRAR (SEQ ID NO: 302) |
| DHD261 | DEERELREVLRKHHRVVREWTKVVEELKRVVELLKRGETSEEDLLRVLKKL |
| | LEMDKRILEVNREVIRVLEKRLT (SEQ ID NO: 303) |
| DHD262 | SLEEIIEELVELVRRSVEIAKESDEVARRIVESEDKKKELIDTLRDLHREW |
| | QEVTKRAEELVREAEKEVR (SEQ ID NO: 304) |
| DHD263 | TAEELLEVHKKSDRVTKEHLRVSEEILKVVEVLTRGEVSSEVLKRVLRKLE |
| | ELTDKLRRVTEEQRRVVEKLN (SEQ ID NO: 305) |
| DHD264 | DLEDLLRRLRRLVDEQRRLVEELERVSRRLEKAVRDNEDERELARLSREHS |
| | DIQDKHDKLAREILEVLKRLLERTE (SEQ ID NO: 306) |
| DHD265 | PEDDVVREIKEDLESNREVLREQKEIHRILELVTRGEVSEEAIDRVLKRQE |
| | DLLKKQKESTDKARKVVEERR (SEQ ID NO: 307) |
| DHD266 | DEVRLITEWLKLSEESTRLLKELVELTRLLRNNVPNVEEILREHERISREL |
| | ERLSRRLKDLADKLERTRR (SEQ ID NO: 308) |
| DHD267 | DEDEVVKVHEEHVKSHEEIHRSHEEVVRAAEEDKRDSRELRTLMEEHRKLL |
| | EENEKSIEEVKKIHERVKR (SEQ ID NO: 309) |
| DHD268 | KKEELIDISKEVLDLDDEINKISKEILELIKKLLRLKEEGREDKDKAREVK |
| | RRIRELHRRIQELNKRLRELHKRVQETKR (SEQ ID NO: 310) |
| DHD269 | PEEDIARRVEDLLRKSEELIKESEKILKESKRLLDRNDSDKRVLETNLRLI |
| | DKHTKLLERNLELLEELLKLAEDVAK (SEQ ID NO: 311) |
| DHD270 | RFKDLSREYIEVVKRLLELSREALEVLREIKDTCKTDKKRIKELIDRLRKL |
| | IEEYKRIIDRLRKLSKDLEEEHR (SEQ ID NO: 312) |
| DHD271 | DEEELVKELKELQRLSEESLEINKRLVEILRLLRRGEVPKEEVEKKLREIK |
| | KEçEKLDREHEKIKKRIEEITK (SEQ ID NO: 313) |
| DHD272 | SLKEKILEIIERNMKLVELSNRSVEIVARILKGEKDDEETLERLLREWDKI |
| | TRDYEEIIKESRKLVELEEEAK (SEQ ID NO: 314) |
| DHD273 | SKTEILRKALEIHKEQIDIVRKLIELSEEVLKLVEESKEKNLEKLKRIDEE |
| | TDRLLERLDELHKRLTELAERLK (SEQ ID NO: 315) |
| DHD274 | SDDEARKQLEEMKRRLREVEKKSKRVEERVRELERLVRENREDEDRVLKTL |
| | EDLLRENEKLVRTIERHVREQRELSKEVK (SEQ ID NO: 316) |
| DHD275 | SEEELEKKADELRKLSEEWRKLQEEDKRLSEMVEKGELDLQEVDEHSLRVL |
| | ERATEVHRTVDKVIEEILRTIN (SEQ ID NO: 317) |
| DHD276 | SEKERHRESQETQEEIRRTHEEIIRKLEEILRRAKAGELPEETLDRLRRIM |
| | ERLKELSERLDDLVRKLRDDHRREQK (SEQ ID NO: 318) |
| DHD277 | SEKEILEELKRILKRVKDISDRLEELDKRTEEIARREPTKELVDELVKIHR |
| | DWLRLHEEILKLVDDALKKVEDATK (SEQ ID NO: 319) |
| DHD278 | DLRELLELQREASRLHRELVKLLTELVKKLELIAKGEDIREEDLKRIKERL |
| | EEIKKRSKRIKEESDEIDKKTK (SEQ ID NO: 320) |
| DHD279 | SERELQRELNKIVRRILEIHREVSELHQRAVKLIRENDNSEELEEISRRIE |
| | ELSKELEKLVREHDEIVKTIE (SEQ ID NO: 321) |
| DHD280 | SEREKLDRNDEELKEINKRVEEIKERSDRITEAIEKNERSEEEIRRLSREQ |
| | NEALQRLLELHKKLVKLHRELLEDTR (SEQ ID NO: 322) |
| DHD281 | DKEDVIRVHDEQHKLIEEQLELTRRIAELVREIAKNTASEEEIKEMLKEIK |
| | RLDDRSREIQDRLQKLLEEIRRKTK (SEQ ID NO: 323) |
| DHD282 | TEEEIVELNKDIQRKSKEHIDLQNELVKKIERAIRENNITEELLEELERLL |
| | RESEKIVEEIRRITDIRKDAK (SEQ ID NO: 324) |
| DHD283 | SEKEILERLLRLSKEQNEISEEIHRLTERLVELKRRKDDDERLKRILDRQK |
| | RLVERAREISKEYEDLLRKLE (SEQ ID NO: 325) |
| DHD284 | SMEELLRKNARLSRKQLKIIDEHLELSTKLTRGEAGDETLEEIERRSREML |
| | EEQRRVDEESKRIREKLK (SEQ ID NO: 326) |
| DHD285 | SEEEIRDIVEKLLRTHEEVLKEIKKLLDDSERVRRRELDKKDLDRIQKEQR |
| | DICEENKEKAKRFDELVKELKKAAK (SEQ ID NO: 327) |
| DHD286 | SEEEHRREMEKVEKEVRDIKRRSEEVKKKVKANTLSEEDLVRLLERLVEDH |
| | KRLQDLSQEIIERDEKATK (SEQ ID NO: 328) |
| DHD287 | DEDELAKEIEDVQRRNKESQEEHDKSVKKLEAAERGEIDEDSLLRVLEEDI |
| | KVLEKDIEVLERSIEVIEKAE (SEQ ID NO: 329) |
| DHD288 | SEKELIRRLLEQQRQHLRLSERLIELSRRLVEVVRKGKDNRDLLRELKKLS |
| | EEHKKHSKDDHEKVREIREREK (SEQ ID NO: 330) |
| DHD289 | DRKDLLKRNIKLLDRALKILDTILKLLEKLSELLKKSSSEEVVKEYKKILD |
| | EIRKLLEESKEIHKESKEILERES (SEQ ID NO: 331) |
| DHD290 | DEEKLIERSKRLQEESEQLLEKFEQILRELTELLEKPDSEELARKIKKLHD |
| | ELRKIIKRNQELIREHEEILRKRD (SEQ ID NO: 332) |
| CC-AI33I3a | GEIAALEKEIAALEWEIAALEQGS (SEQ ID NO: 333) |
| CC-AI33I3b | GKIAALEYKIAALKKKIAALKQGS (SEQ ID NO: 334) |
| CC-AN3.5BN3a | GLEQEIAALEKENAALEWEIAALEQGG (SEQ ID NO: 335) |
| CC-AN3.5BN3b | GKIAALEYKNAALKKKIAALKQGG (SEQ ID NO: 336) |
| CC- | GLEQEIAALEKENAALEWEIAALEQGG (SEQ ID NO: 335) |
| AN3.5BN3.5a | |
| CC- | GLKQKIAALEYKNAALKKKIAALKQGG (SEQ ID NO: 337) |
| AN3.5BN3.5b | |
| CC-AN3.5BN4a | GLEQEIAALEKENAALEWEIAALEQGG (SEQ ID NO: 335) |
| CC-AN3.5BN4b | GKIAALKQKIAALEYKNAALKKKIAALKQGG (SEQ ID NO: 338) |
| CC-AN33N3a | GEIAALEKENAALEWEIAALEQGG (SEQ ID NO: 339) |
| CC-AN33N3b | GKIAALEYKNAALKKIAALKQGG (SEQ ID NO: 336) |
| CC-AN33N3.5a | GEIAALEKENAALEWEIAALEQGG (SEQ ID NO: 339) |
| CC-AN33N3.5b | GLKQKIAALEYKNAALKKKIAALKQGS (SEQ ID NO: 340) |
| CC-AN33N4a | GEIAALEKENAALEWEIAALEQGG (SEQ ID NO: 339) |
| CC-AN33N4b | GKIAALKQKIAALEYNAALKKKIAALKQGS (SEQ ID NO: 341) |
| CC-AN43N3a | GEIAALEQEIAALEKENAALEWEIAALEQGG (SEQ ID NO: 342) |
| CC-AN43N3b | GKIAALEYKNAALKKKIAALKQGG (SEQ ID NO: 336) |
| CC-AN43N3.5a | GEIAALEQEIAALEKENAALEWEIAALEQGG (SEQ ID NO: 342) |
| CC-AN43N3.5b | GLKQKIAALEYKNAALKKKIAALKQGG (SEQ ID NO: 337) |
| CC-AN43N4a | GEIAALEQEIAALEKENAALEWEIAALEQGG (SEQ ID NO: 342) |
| CC-AN43N4b | GKIAALKQKIAALEYKNAALKKKIAALKQGG (SEQ ID NO: 338) |
| IAAL E3 | EIAALEKEIAALEKEIAALEK (SEQ ID NO: 343) |
| IAAL K3 | KIAALKEKIAALKEKIAALKE (SEQ ID NO: 344) |
| IAAL E4 | EIAALEKEIAALEKEIAALEKEIAALEK (SEQ ID NO: 345) |
| IAAL K4 | KIAALKEKIAALKEKIAALKEKIAALKE (SEQ ID NO: 346) |
| ISAL E3 | EISALEKEISALEKEISALEK (SEQ ID NO: 347) |
| ISAL K3 | KISALKEKISALKEKISALKE (SEQ ID NO: 348) |
| ISAL E4 | EISALEKEISALEKEISALEKEISALEK (SEQ ID NO: 349) |
| ISAL K4 | KISALKEKISALKEKISALKEKISALKE (SEQ ID NO: 350) |
| VAAL E3 | EVAALEKEVAALEKEVAALEK (SEQ ID NO: 351) |
| VAAL K3 | KVAALKEKVAALKEKVAALKE (SEQ ID NO: 352) |
| VAAL E4 | EVAALEKEVAALEKEVAALEKEVAALEK (SEQ ID NO: 353) |
| VAAL K4 | KVAALKEKVAALKEKVAALKEKVAALKE (SEQ ID NO: 354) |
| VSAL E3 | EVSALEKEVSALEKEVSALEK (SEQ ID NO: 355) |
| VSAL K3 | KVSALKEKVSALKEKVSALKE (SEQ ID NO: 356) |
| VSAL E4 | EVSALEKEVSALEKEVSALEKEVSALEK (SEQ ID NO: 357) |
| VSAL K4 | KVSALKEKVSALKEKVSALKEKVSALKE (SEQ ID NO: 358) |
| ACID | AQLEKELQALEKENAQLEWELQALEKELAQ (SEQ ID NO: 359) |
| BASE | AQLKKKLQALKKKNAQLKWKLQALKKKLAQ (SEQ ID NO: 360) |
| SAFa | KIAALKQKIASLKQ (SEQ ID NO: 361) |
| SAFb | EIDALEYENDALEQ (SEQ ID NO: 362) |
| SAFC | KIRRLKQKNARLKQ (SEQ ID NO: 363) |
| SAFd | EIAALEYEIAALEQ (SEQ ID NO: 364) |
| C3A | EIRRLEEEIRRLEEENRRLEY (SEQ ID NO: 365) |
| C3B | RNEELRYRIEELRRRIEELRR (SEQ ID NO: 366) |
| C4A | EIRRLEEEIRRLEEEIRRLEEENRRLEY (SEQ ID NO: 367) |
| C4B | RIEELRRRNEELRYRIEELRRRIEELRR (SEQ ID NO: 368) |
| C3AK | EIKKLEEEIKKLEEENKKLEY (SEQ ID NO: 369) |
| C3BK | KNEELKYKIEELKKKIEELKK (SEQ ID NO: 370) |
| C4AK | EIKKLEEEIKKLEEEIKKLEEENKKLEY (SEQ ID NO: 371) |
| C4BK | KIEELKKKNEELKYKIEELKKKIEELKK (SEQ ID NO: 372) |
| K-coil | KVSALKEKVSALKEKVSALKEKVSALKEKVSALKEG (SEQ ID |
| | NO: 373) |
| E-coil | EVSALKEKVSALEKEVSALKEKVSALEKEVSALEKGG (SEQ ID |
| | NO: 374) |

In some embodiments, the fusion protein may be no more than 100 aa in length, e.g., fusion protein is 40-100 amino acids in length, e.g., 50-80 amino acids in length. The fusion protein reduced to practice in the examples section is 55 amino acids in length. However, this molecule could be shortened by reducing the size or eliminating the linker and potentially removing one of the heptads from the alpha-helix.

In some embodiments, the fusion protein may contain a linker (e.g., of 2-20 amino acids) between the heterodimerization domain and the C-terminal degron. For example, the fusion protein could contain a flexible linker, many examples of which are known.

In some embodiments, the amino acid sequence of the fusion protein may be at least 80%, at least 90% or at least 95% identical to the “synZIP STUD” sequence presented below, with the linker sequence, without the linker sequence, with a longer linker sequence (e.g., a flexible linker of greater than 10 amino acids), or with a shorter linker sequence (e.g., flexible linker of less than 10 amino acids (e.g., 2, 3, 4, 5, 6, 7, 8 or 9 amino acids).

In addition, the fusion protein may contain a targeting domain, e.g., a transmembrane domain, that is N-terminal to the heterodimerization domain. In the case of a transmembrane domain, the degron end of the fusion protein will be intracellular. This embodiment will tether the degron to the plasma membrane, which is particularly useful for targeting transmembrane receptors for degradation. The fusion protein may additionally contain a nuclear localization sequence or other targeting sequence, as desired.

In any embodiment, the fusion partner may comprise amino acids 1-41 or all of SEQ ID NO: 413.

In embodiments in which the fusion protein does not contain a transmembrane domain (i.e., embodiments in which the protein is soluble) the entire fusion protein may be lysine free. In embodiments in which the fusion protein contains a transmembrane domain (i.e., embodiments in which the protein is tethered to the plasma membrane) only the intracellular part of the fusion protein needs to be lysine free. In these embodiments, the intracellular part of the fusion protein (which contains the dimerization domain and stud) may be no more than 100 aa in length, e.g., 40-100 or amino acids in length, e.g., 50-80 amino acids in length, including or excluding any flexible linker sequence.

The fusion protein may be designed or modified so that there are no lysines in the entire fusion protein (if the fusion protein is soluble) or in the intracellular part of the fusion protein (if it contains a transmembrane domain), thereby protecting the fusion protein from cis-ubiquitination and subsequent auto-degradation. In these embodiments, this domain may be designed by identifying lysines, and then changing the lysines to another residue (e.g., arginine, which is similar to lysine but not targeted by the ubiquitin ligase). In some embodiments, all of the lysines of the fusion protein may be modified to be arginines. In these embodiments, the fusion protein may be lysine free. In other embodiments, a subset of lysines (e.g., 1, 2, 3, 4, 5, 6 or 7 lysines) may be mutated to tune the balance of cis-versus trans-ubiquitination. This strategy may be useful for tuning the activity of the fusion protein. In other cases, the number of heptad repeats may be altered (e.g., reduced) which should decrease the affinity for the protein to its target without decreasing specificity.

The C-terminal degron may be RRRGN (SEQ ID NO:375) or RRRG (SEQ ID NO: 32), where the respective N and G amino acids in these sequences define the C terminus of the protein (see, e.g., Bonger, Nat. Chem Biol 7, 531-537). A shorter degron (e.g., a degron of 4, 5 or 6 amino acids) can be used in some embodiments. Other sequence could be used, however, including any of the C-terminal degron sequences/motifs from the following table.

TABLE 2

| Name | Sequence | ID NO | Motif |
|---|---|---|---|
| fRA68_EMID1 | RGKRGGHATNYRIVAPRSRDERG* | 1 | RG* |
| | (SEQ ID NO: 376) | | |
| fRA69_CHGA | ESLSAIEAELEKVAHQLQALRRG* | 2 | RG* |
| | (SEQ ID NO: 377) | | |
| fRA70 MAGEA3 | KISGGPHISYPPLHEWVLREGEE* | 3 | EE* |
| | (SEQ ID NO: 378) | | |
| fRA71_MAGEA3EEtoAA | KISGGPHISYPPLHEWVLREGAA* | 4 | EE* to Ax/A* |
| | (SEQ ID NO: 379) | | |
| fRA72_PIK3C2B | LRELDLAQEKTGWFALGSRSHGTL* | 5 | RxxGxx* |
| | (SEQ ID NO: 380) | | |
| fRA73_PXN | LRELDLAQEKTGWFALGSRHCGRT* | 6 | RxxGxx* |
| | (SEQ ID NO: 381) | | |
| fRA74_Peptide35 | YKKAGSGIPLRMNSLFRKRNKGK* | 7 | RxxGxx* |
| | (SEQ ID NO: 382) | | |
| fRA75_CDK5R1 | VFSDLKNESGQEDKKRLLLGLDR* | 8 | R* motif, |
| | (SEQ ID NO: 383) | | |
| fRA76_CDK5R1trunc | VFSDLKNESGQEDKKRLLLGLD* | 9 | R truncated, |
| | (SEQ ID NO: 384) | | |
| fRA77_SIL1 | DGEDEGYFQELLGSVNSLLKELR* | 10 | R* |
| | (SEQ ID NO: 385) | | |
| fRA78_SIL1trunc | DGEDEGYFQELLGSVNSLLKEL* | 11 | R truncated, |
| | (SEQ ID NO: 386) | | |
| fRA79_N-Myc | LEKEKLQARQQQLLKKIEHARTC* | 12 | Rxx* |
| | (SEQ ID NO: 387) | | |
| fRA80_N-Myctrunc | LEKEKLQARQQQLLKKIEHA* (SEQ | 13 | Rxx* to A*, |
| | ID NO: 388) | | |
| fRA81_MSRB2 | GPGPNGQRFCINSAALKFKPRKH* | 14 | Rxx* |
| | (SEQ ID NO: 389) | | |
| fRA82_OR4C13 | LRNAQMKNAIRKLCSRKAISSVK* | 15 | Vx* motif |
| | (SEQ ID NO: 390) | | |
| fRA83_OR4C13Dmut | LRNAQMKNAIRKLCSRKAISSDK* | 16 | Vx to Dx |
| | (SEQ ID NO: 391) | | |
| fRA84_SREBF2 | RRSCNDCQQMIVKLGGGTAIAAS* | 17 | Axx |
| | (SŁQ ID NO: 392) | | |
| fRA85_SREBF2-Dmut | RRSCNDCQQMIVKLGGGTAIADS* | 18 | Ax |
| | (SEQ ID NO: 393) | | |
| fRA86_CPS1 | QKSRKVDSKSLFHYRQYSAGKAA* | 19 | AA* |
| | (SEQ ID NO: 394) | | |
| fRA87_CPS1DDmut | QKSRKVDSKSLFHYRQYSAGKDD* | 20 | AA to DD, |
| | (SEQ ID NO: 395) | | |
| fRA88_CPS1CText | QKSRKVDSKSLFHYRQYSAGKAAKAST | 21 | AA Ct |
| | N* (SEQ ID NO: 396) | | |
| fRA89_EPHB2 | REIQGIFFKEDSHKESNDCSCGG* | 22 | GG |
| | (SEQ ID NO: 397) | | |
| fRA90_PDGFC | SLTDVALEHHEECDCVCRGSTGG* | 23 | GG |
| | (SEQ ID NO: 398) | | |
| fRA91_ASCC3 | RRLDGKEEDEKMSRASDRFRGLR* | 24 | RG/R* dual |
| | (SEQ ID NO: 399) | | |
| RA2102_degBon1 | TRGVEEVAEGVVLLRRRGN* (SEQ | 25 | Rxx*/RxxG |
| | ID NO: 400) | | |
| RA2106_Clone1 | (GIPLR)NLGIR* (SEQ ID | 26 | RG/R* dual |
| | NO: 401) | | |
| RA2107_Clone6 | (GIPLR)QRKLQRTSRG* (SEQ ID | 27 | RG* |
| | NO: 402) | | |
| RA2108_Clone6GtoA | (GIPLR)QRKLQRTSRA* (SEQ ID | 28 | RG* to A*, |
| | NO: 403) | | |
| RA2109_Clone8 | (GIPLR)PHKRLLKGSQYG* (SEQ | 29 | RG*-like |
| | ID NO: 404) | | |

Also provided is a recombinant nucleic acid comprising a promoter and a coding sequence that encodes the fusion protein, wherein promoter and coding sequence are operably linked. In these embodiments, the promoter may be chemically-inducible, tissue-specific, cell state-responsive, or activated by an external stimulus that is recognized at the plasma membrane. In some embodiments, the promoter is not constitutive, meaning that the fusion protein is not expressed unless there is a perturbation in the cells. For example, in some embodiments, the promoter may be responsive to the state of the cell. In this example, the promoter may be active if the cell changes identity (e.g., differentiates in the case of a stem cell) or the promoter may become more active if a new signaling pathway is activated (which would be the case for T cell activation or T cell exhaustion, for example). In some cases, fusion protein may be induced if a receptor on the cell (e.g., a proteolytic receptor) binds to an antigen on another cell. In these embodiments, the binding event may induce a cleavage event that, in turn, releases a transcriptional activator that activates expression of the fusion protein.

A cell, e.g., a eukaryotic cell, containing the nucleic acid is also provided. The cell may be in vivo, ex vivo, or in vitro and, in some embodiments, the cell may be a therapeutic cell (e.g., a recombinant immune cell such as a CAR T, a Treg cell or stem cell). The nucleic acid may integrated into the genome of the cell or exogenous to the genome of the cell.

As would be apparent, the cell may further comprise a recombinant target protein, where the target protein comprises a binding partner for the lysine-free alpha-helical heterodimerization domain. The target protein can be virtually any protein but in particular embodiments the protein can be a recombinant transcription factor, enzyme, kinase, receptor or cytokine.

The binding partner for the lysine-free alpha-helical heterodimerization domain does not itself have to be lysine free and, in many cases, it may be advantageous for the binding partner in the fusion protein to have one or more (e.g., 1, 2, 3, 4, or 5 or more) lysines because those amino acids will be ubiquitinated, thereby targeting the target protein for degradation.

Binding partners for alpha-helical heterodimerization domains listed above are listed in the table below. As with the alpha-helical heterodimerization domains listed above, the binding partners may be 28-80 amino acids in length and in some embodiments may be composed of a single bZIP domain (which are typically 28-50 in length) or a “designed heterodimer” (which are typically longer at 50-80 amino acids in length). The binding partners may have a similar length to the alpha-helical heterodimerization domain, although this is not required.

TABLE 3

| name | Interactor (s) |
|---|---|
| bZIP c-Jun | Fos, ATF4, C/EBPA |
| bZIP JUN3 | C/EBPG |
| bZIP Fos | c-Jun, C/EBPA, C/EBPG, C/EBPE |
| bZIP ATF3 | 3AFT2 |
| bZIP ATF4 | C/EBPG, CHOP |
| bZIP ATF5 | 3AFT3, MAFB |
| bZIP C/E3PA | c-Jun, Fos, C/EBPB, BAFT |
| bZIP C/E3PG | ATF4, Fos, JUNB |
| bZIP C/E3PE | Fos |
| bZIP CHO? | ATF 4 |
| bZIP CRE3 | NFIL3 |
| bZIP NFIL3 | CREB, BAFT3 |
| bZIP BATE | HILF, C/EBPA |
| bZIP BATE2 | ATF 3 |
| bZip BATE3 | NFIL3, ATF5 |
| bZIP HLF | 3ATF |
| bZIP MAF3 | ATF5 |
| SYNZIP1 | SYNZIP2 |
| SYNZIP2 | SYNZIP1, SYNZIP14, SYNZIP19, |
| | SYNZIP20 |
| SYNZIP3 | SYNZIP4, |
| SYNZIP4 | SYNZIP3, SYNZIP21 |
| SYNZIP5 | SYNZIP6, SYNZIP16, SYNZIP21 |
| SYNZIP6 | SYNZIP5, SYNZIP19, SYNZIP20, |
| | SYNZIP21 |
| SYNZIP7 | |
| SYNZIP8 | |
| SYNZIP9 | |
| SYNZIP10 | |
| SYNZIP11 | SYNZIP19, SYNZIP20, SYNZIP21 |
| SYNZIP12 | |
| SYNZIP13 | |
| SYNZIP14 | SYNZIP17, SYNZIP21 |
| SYNZIP15 | |
| SYNZIP16 | SYNZIP5, SYNZIP19, SYNZIP20, |
| | SYNZIP21 |
| SYNZIP17 | SYNZIP18, SYNZIP21, |
| SYNZIP18 | SYNZIP17, SYNZIP19, SYNZIP20 |
| SYNZIP19 | SYNZIP6, SYNZIP11, SYNZIP16, |
| | SYNZIP18, SYNZIP21 |
| SYNZIP20 | SYNZIP2, SYNZIP6, SYNZIP11, |
| | SYNZIP16, SYNZIP18, SYNZIP21 |
| SYNZIP21 | SYNZIP4, SYNZIP5, SYNZIP6, |
| | SYNZIP11, SYNZIP14, SYNZIP16, |
| | SYNZIP17, SYNZIP19, SYNZIP20 |
| SYNZIP22 | |
| CC-AI3BI3a | CC-AI3BI3b |
| CC-AI3BI3b | CC-AI3BI3a |
| CC-AN3.53N3a | CC-AN3.5BN3b |
| CC-AN3.53N3b | CC-AN3.5BN3a |
| CC- | CC-AN3.5BN3.5b |
| AN3.5BN3.5a | |
| CC- | CC-AN3.5BN3.5a |
| AN3.5BN3.5b | |
| CC-AN3.53N4a | CC-AN3.5BN4b |
| CC-AN3.53N4b | CC-AN3.5BN4a |
| CC-AN3BN3a | CC-AN3BN3b |
| CC-AN3BN3b | CC-AN3BN3a |
| CC-AN3BN3.5a | CC-AN3BN3.5b |
| CC-AN3BN3.5b | CC-AN3BN3.5a |
| CC-AN3BN4a | CC-AN3BN4b |
| CC-AN3BN4b | CC-AN3BN4a |
| CC-AN4BN3a | CC-AN4BN3b |
| CC-AN4BN3b | CC-AN4BN3a |
| CC-AN4BN3.5a | CC-AN4BN3.5b |
| CC-AN4BN3.5b | CC-AN4BN3.5a |
| CC-AN4BN4a | CC-AN4BN4b |
| CC-AN4BN4b | CC-AN4BN4a |
| ZAAL E3 | IAAL K3 |
| ZAAL K3 | IAAL E3 |
| ZAAL E4 | IAAL K4 |
| ZAAL K4 | IAAL E4 |
| ISAL E3 | ISAL K3 |
| SAL K3 | ISAL E3 |
| ISAL E4 | ISAL K4 |
| ISAL K4 | ISAL E4 |
| VAAL E3 | VAAL K3 |
| VAAL K3 | VAAL E3 |
| VAAL E4 | VAAL K4 |
| VAAL K4 | VAAL E4 |
| VSAL E3 | VSAL K3 |
| VSAL K3 | VSAL E3 |
| VSAL E4 | VSAL K4 |
| VSAL K4 | VSAL E4 |
| ACID | 3ASE |
| BASE | ACID |
| SAFa | SAFC |
| SAFb | SAFd |
| SAFc | SAFa |
| SAFd | SAFb |
| C3A | C3B |
| C3B | C3A |
| C4A | C4B |
| C4B | C4A |
| C3AK | C3BK |
| C3BK | C3AK |
| C4AK | C4BK |
| C4BK | C4AK |
| K-coil | E-coil |
| E-coil | K-coil |

For the DHD sequences, each consecutive pair of sequences (DHD1 and DHD2, DHD3 and DHD4, DHD5 and DHD6) etc., should heterodimerize.

Also provided is a method for degrading a target protein comprising inducing expression of the fusion protein in a cell that contains the target protein, and a method of treatment comprising: administering a cell that contains the nucleic acid and contains the target protein to an individual in need thereof. In any of these methods, the method may further comprising inhibiting degradation of the target protein by a proteasome inhibitor.

Various optional components, features and elements are described below, along with further details.

### Alpha-Helical Heterodimerization Domains

As noted above, the fusion protein has an alpha-helical heterodimerization domain, which term is intended to refer to bZIP domains (including synZIPs), helix-loop-helix domains (see, e.g., Chen et al (Nature 2019 565: 106-111) and US20210355175A1) and other binding pairs that specifically bind to one another via an alpha-helix. As is well known, a leucine zipper is formed by amphipathic interaction between two bZIP domains. In a bZIP domain, the amino acids are spaced out in each region's polypeptide sequence in such a way that when the sequence is coiled in a 3D alpha-helix, the leucine residues line up on the same side of the helix. This region of the alpha-helix-containing the leucines which line up-is called a bZIP domain, and leucines from each ZIP domain can weakly interact with leucines from other ZIP domains, reversibly holding their alpha-helices together (dimerization). When these alpha helices dimerize, the zipper is formed. The hydrophobic side of the helix forms a dimer with itself or another similar helix, burying the non-polar amino acids away from the solvent. The hydrophilic side of the helix interacts with the water in the solvent.

Leucine zippers are a type of coiled coil, which is composed of two or more alpha helices that are wound around each other to form a supercoil. Coiled coils and bZIPs contain 3- and 4-residue repeats whose hydrophobicity pattern and residue composition is compatible with the structure of amphipathic alpha-helices. The alternating three-and four- residue sequence elements constitute heptad repeats in which the amino acids are designated from a′ to g′ (Hodges et al Cold Spring Harbor Symposia on Quantitative Biology. 1973 37: 299-310). While residues in positions a and d are generally hydrophobic and form a zigzag pattern of knobs and holes that interlock with a similar pattern on another strand to form a tight-fitting hydrophobic core, residues in positions e and g are charged residues contributing to the electrostatic interaction (Mason ChemBioChem. 2004 5 (2): 170-6)

In the case of bZIP domains or leucine zippers, leucines are predominant at the d position of the heptad repeat. These residues pack against each other every second turn of the alpha-helices, and the hydrophobic region between two helices is completed by residues at the a positions, which are also frequently hydrophobic.

Coiled-coils can be predicted using primary amino acid sequence alone using a variety of methods. See, e.g., Delorenzi et al (Bioinformatics 2002 18:617-25), Berger et al (Proc Natl Acad Sci 1995 92: 8259-63), Wolf et al (Protein Sci. 1997 6: 1179-89) and Lupas et al (Curr. Opin. Struct. Biol. 1997 7:388-93), among many others (e.g., Chen et al, supra).

Numerous examples of alpha-helical heterodimerization domains are known in the art or can be readily derived from proteins that interact with one another via those domains. The sequences listed above are examples.

### Linkers

In some embodiments, the fusion protein may further comprise (c), a linker, between the target-binding domain of (a) and the degradation domain of (b). A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

Suitable linkers can be readily selected and can be any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers (including, for example, (GS)n, (GSGGS)n (SEQ ID NO:405) and (GGGS)n (SEQ ID NO: 406), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:407), GGSGG (SEQ ID NO:408), GSGSG (SEQ ID NO:409), GSGGG (SEQ ID NO: 410), GGGSG (SEQ ID NO:411), GSSSG (SEQ ID NO:412), and the like.

### Transmembrane Domains

In some embodiments, the fusion protein may have a transmembrane domain. In these embodiments, the fusion protein may target a transmembrane protein such as a CAR, SynNotch, a receptor, or any other protein that is located on the plasma membrane of a mammalian cell (see, e.g., Sharpe et al, Cell. 2010 142: 158-169). In these embodiments, the fusion protein may comprise: (a) a transmembrane domain, (b) a target-binding domain that binds to an intracellular site in a transmembrane protein (such as a CAR, SynNotch or a receptor); and (c) a degradation domain that is heterologous to the target-binding domain, wherein the degradation domain is a degron or E3 ligase-recruiting domain, as discussed above. When the protein is expressed in a mammalian cell, the target-binding domain and degradation domain are intracellular, but tethered to the plasma membrane via the transmembrane domain. In the cell, binding of the fusion protein to the transmembrane protein via the target-binding domain induces degradation of the transmembrane protein, as discussed above. As would be apparent, the nucleic acid encoding such a fusion protein may additionally comprise a signal peptide. Suitable transmembrane domains include those of CD8, CD4, CD3 zeta, CD28, CD134, CD7, although there are thousands of others that one could use. The transmembrane domain can be C-terminal or N-terminal, or anywhere in the fusion protein depending on the other components of the protein used.

In these embodiments, the fusion protein can be used to controllably target a membrane protein such as a CAR or SynNotch in a CAR T cell. In some embodiments, the fusion protein could be part of a circuit that controls the expression of a CAR. For example, expression of the fusion protein could be induced by binding of a first antigen to a binding triggered transcriptional switch such as a SynNotch. After the switch is activated, the fusion protein is expressed, and the fusion protein degrades a CAR in the cell. This way, the CAR expression can be “switched off” by binding of the SynNotch to the first antigen.

### Target Proteins

The target protein can be any protein that has been engineered to contain a binding partner for the alpha-helical heterodimerization domain. Examples of target proteins include, but are not limited to, transcriptional activators, transcriptional repressors, transcriptional co-activators, transcriptional co-repressors, DNA binding polypeptides, RNA binding polypeptides, translational regulatory polypeptides, hormones, cytokines, toxins, antibodies, chromatin modulators, suicide proteins, organelle specific polypeptides (e.g., a nuclear pore regulator, a mitochondrial regulator, an endoplasmic reticulum regulator, and the like), pro-apoptosis polypeptides, anti-apoptosis polypeptides, other polypeptides that promote cell death through other mechanisms, pro-proliferation polypeptides, anti-proliferative polypeptides, immune co-stimulatory polypeptides, site-specific nucleases, recombinases, inhibitory immunoreceptors, an activating immunoreceptor, Cas9 and variants of RNA targeted nucleases, and DNA recognition polypeptides, dominant negative variants of a polypeptide, a signaling polypeptide, a receptor tyrosine kinase, a non-receptor tyrosine kinase, a polypeptide that promotes differentiation, enzymes, structural proteins, and the like.

For example, in some embodiments, the target protein may be a therapeutic protein that, when expressed on the surface of an immune cell, activates the immune cell or inhibits activation of the immune cell when it binds to an antigen on the diseased cell. In these embodiments, the therapeutic protein may be an immune receptor (e.g., a chimeric antigen receptor (CAR), a T cell receptor (TCR), or an iCAR, etc) that contains an extracellular antigen binding domain and an intracellular a binding partner for the alpha-helical heterodimerization domain (among other domains). In these embodiments, the fusion protein may comprise: (a) a lysine-free alpha-helical heterodimerization domain that binds to the binding partner in the immune receptor and (b) a C-terminal degron, wherein, in the therapeutic cell, binding of the fusion protein to a target protein via the target-binding domain induces degradation of the immune receptor protein. In these embodiments, the fusion protein may additionally contain a transmembrane domain. In some cases, the therapeutic protein may be an inhibitory immune cell receptor (iICR) such as an inhibitory chimeric antigen receptor (iCAR), wherein binding of the iICR to the third antigen inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments, an inhibitory immunoreceptor may comprise an intracellular immunoreceptor tyrosine-based inhibition motif (ITIM), an immunoreceptor tyrosine-based switch motif (ITSM), an NpxY motif, or a YXXΦ motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188:2957; and Baitsch et al. (2012) PLoSOne 7: e30852.

In some embodiments, therapeutic protein may be an antigen-specific therapeutic that is secreted from the cell. For example, the antigen-specific therapeutic may be an antibody that binds to an immune checkpoint inhibitor e.g., an antibody that binds to PD1, PD-L1, PD-L2, CTLA4, TIM3, LAG3 or another immune checkpoint. Alternatively, the secreted antigen-specific therapeutic may be a bioactive peptide such as a cytokine (e.g., Il-1ra, IL-4, IL-6, IL-10, IL-11, IL-13, or TGF-β, among many others). In some embodiments, the secreted protein may be an enzyme, e.g., a superoxide dismutase for removing reactive oxygen species, or a protease for unmasking a protease activatable antibody (e.g., a pro-body) in the vicinity of a cancer cell. In these cases, the target protein may be degraded before it is secreted.

Alternatively, the therapeutic protein may be a protein that, when expressed, is internal to the cell, such as a SLP76, ZAP70, or Cas9 protein.

In any embodiment, the fusion protein may contain a localization signal (e.g., a nuclear localization sequence) in order to facilitate translocation of the fusion into a cell compartment (e.g., the nucleus).

In any embodiment, expression of the fusion protein may be inducible, tissue-specific, or constitutive. This may be done by operably linking the coding sequence for the fusion protein to an appropriate promoter.

The therapeutic cell may be genetically modified to contain a nucleic acid comprising an expression cassette comprising a promoter and a coding sequence for the fusion protein as described above. The therapeutic cell may be an immune cell or stem cell, for example, and the nucleic acid may be introduced into the cell by various means, including e.g., through the use of a viral vector.

As noted above, in some embodiments, the therapeutic cell may also express a therapeutic protein, where the therapeutic protein may be on the surface of the cell, secreted by the cell, or on the inside of the cell (e.g., in the cytoplasm or nucleus of the cell).

### Therapeutic Cells

In some instances, a therapeutic cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or a progenitor thereof, obtained from an individual. As another example, the cell is a cytotoxic cell, or a progenitor thereof, obtained from an individual. As another example, the cell is a stem cell or progenitor cell obtained from an individual.

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+ T cell). In some cases, the cell is a helper T cell (e.g., a CD4+ T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+ Treg cell. In some cases, the cell is a CD4+, FOXP3− Treg cell. The immune cell can be immunostimulatory or immunoinhibitory.

In some embodiments, the therapeutic cell may be a CAR T cell. In these embodiments, the cell may be a T cell that expresses a CAR, where the CAR comprises an extracellular domain, a transmembrane region and an intracellular signaling domain; where the extracellular domain comprises a ligand or a receptor and the intracellular signaling domain comprises an ITAM domain, e.g., the signaling domain from the zeta chain of the human CD3 complex (CD3zeta), and, optionally, one or more costimulatory signaling domains, such as those from CD28, 4-1BB and OX-40. The extracellular domain contains a recognition element (e.g., an antibody or other target-binding scaffold) that enables the CAR to bind a target. In some cases, a CAR comprises the antigen binding domains of an antibody (e.g., an scFv) linked to T-cell signaling domains. In some cases, when expressed on the surface of a T cell, the CAR can direct T cell activity to those cells expressing a receptor or ligand for which this recognition element is specific. As an example, a CAR that contains an extracellular domain that contains a recognition element specific for a tumor antigen can direct T cell activity to tumor cells that bear the tumor antigen. The intracellular region enables the cell (e.g., a T cell) to receive costimulatory signals. The costimulatory signaling domains can be selected from CD28, 4-1BB, OX-40 or any combination of these. Exemplary CARs comprise a human CD4 transmembrane region, a human IgG4 Fc and a receptor or ligand that is tumor-specific, such as an IL13 or IL3 molecule. In these embodiments, activation of a CAR activates the immune cell.

Suitable therapeutic cells also include stem cells, progenitor cells, as well as partially and fully differentiated cells. Suitable cells include neurons; liver cells; kidney cells; immune cells; cardiac cells; skeletal muscle cells; smooth muscle cells; lung cells; and the like.

Suitable cells include a stem cell (e.g. an embryonic stem(ES) cell, an induced pluripotent stem (iPS) cell; a germ cell (e.g., an oocyte, a sperm, an oogonia, a spermatogonia, etc.); and a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

Cells of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, an immune cell, a stem cell, etc., is obtained from an individual; and the cell obtained from the individual is modified to express components of a circuit of the present disclosure. The modified cell can thus be modified with control feedback to one or more signaling pathways of choice, as defined by the one or more molecular feedback circuits present on the introduced nucleic acids. In some cases, the modified cell is modulated ex vivo. In other cases, the cell is introduced into and/or already present in an individual (e.g., the individual from whom the cell was obtained); and the cell is modulated in vivo, e.g., by administering a nucleic acid or vector to the individual in vivo.

In some instances, the cell is obtained from an individual. For example, in some cases, the cell is a primary cell. As another example, the cell is a stem cell or progenitor cell obtained from an individual. Allogenic cells may be used in some cases.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is not an immune cell. In these embodiments, the host cell may be a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, or a stem cell, and the like.

### Nucleic Acids and Promoters

Given that the genetic code is known, sequence that encodes the fusion protein can be readily determined. In some embodiments, the coding sequence may be codon optimized for expression in mammalian (e.g., human or mouse) cells, strategies for which are well known (see, e.g., Mauro et al., Trends Mol. Med. 2014 20: 604-613 and Bell et al Human Gene Therapy Methods 27:6). As would be understood, the coding sequence may be operably linked to a promoter, which may be inducible, tissue-specific, or constitutive. In some embodiments, the promoter may be activated by an engineered transcription factor that is heterologous to the cell, e.g., a Gal4-, LexA-, Tet-, Lac-, dCas9-, zinc-finger-and TALE-based transcription factors.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice). For example, the fusion protein could be induced during T cell exhaustion, in response to binding to an antigen (via a proteolytic receptor), an exogenously added compound, etc. In some embodiments, the promoter is not constitutive.

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

In some cases, the promoter is a cardiomyocyte-specific promoter. In some cases, the promoter is a smooth muscle cell-specific promoter. In some cases, the promoter is a neuron-specific promoter. In some cases, the promoter is an adipocyte-specific promoter. Other cell type-specific promoters are known in the art and are suitable for use herein.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

The cell may be used in a method of treatment that comprises administering the cell to a patient in need thereof.

In some embodiments, the patient may have cancer, e.g., breast cancer, B cell lymphoma, pancreatic cancer, Hodgkin lymphoma cell, ovarian cancer cell, prostate cancer, mesothelioma, lung cancer (e.g., a small cell lung cancer), non-Hodgkin B-cell lymphoma (B-NHL) cell, ovarian cancer, a prostate cancer, melanoma cell, a chronic lymphocytic leukemia cell, acute lymphocytic leukemia cell, a neuroblastoma, a glioma, a glioblastoma, a medulloblastoma, a colorectal cancer, etc. In these embodiments, the therapeutic cell may be a CAR T cell that comprises a CAR that recognizes an antigen expressed by the cancer cells.

In some embodiments, the patient may have an inflammatory condition or autoimmune disease. In these embodiments, the cell may be T-helper cell or a Tregs for use in an immunomodulatory method. Immunomodulatory methods include, e.g., enhancing an immune response in a mammalian subject toward a pathogen; enhancing an immune response in a subject who is immunocompromised; reducing an inflammatory response; reducing an immune response in a mammalian subject to an autoantigen, e.g., to treat an autoimmune disease; and reducing an immune response in a mammalian subject to a transplanted organ or tissue, to reduce organ or tissue rejection.

In some embodiments, the patient is in need of a stem cell transplantation.

### EXAMPLES

The following examples are put forth so as to provide those of ordinary skill in the art with a complete disclosure and description of how to make and use the present invention, and are not intended to limit the scope of what the inventors regard as their invention.

This disclosure provides a new protein degradation technology based on a protein chimera contains a lysine-free heterodimerization domain such as a bZIP domain, an optional linker, and a protein degradation domain, e.g., a degron. This protein chimera is able to recruit the endogenous E3 ligase machinery of the cell to novel targets, triggering the ubiquitination and degradation of recombinant targets that have a partner for the heterodimerization domain. This tool is referred to as a “synthetic targetter of ubiquitination and degradation”, or “STUD” for short. A particularly potent C-terminal minimal degron motif of the sequence RRRG (Arg-Arg-Arg-Gly; also referred to as the “Bonger” motif; SEQ ID NO:32) was used as a basis for developing this technology. In theory, this system should be amenable to a variety of other C-terminal degrons.

### Example 1

### Synthetic Targeter of Ubiquitination and Degradation (‘STUD’) Potently Degrades Fluorescent Protein Targets in All Tested Mammalian Cell Lines

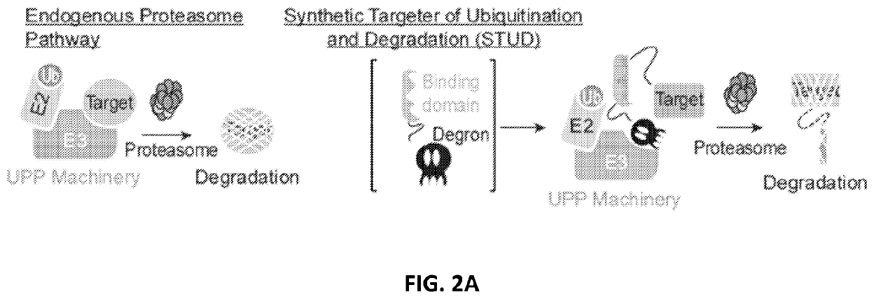

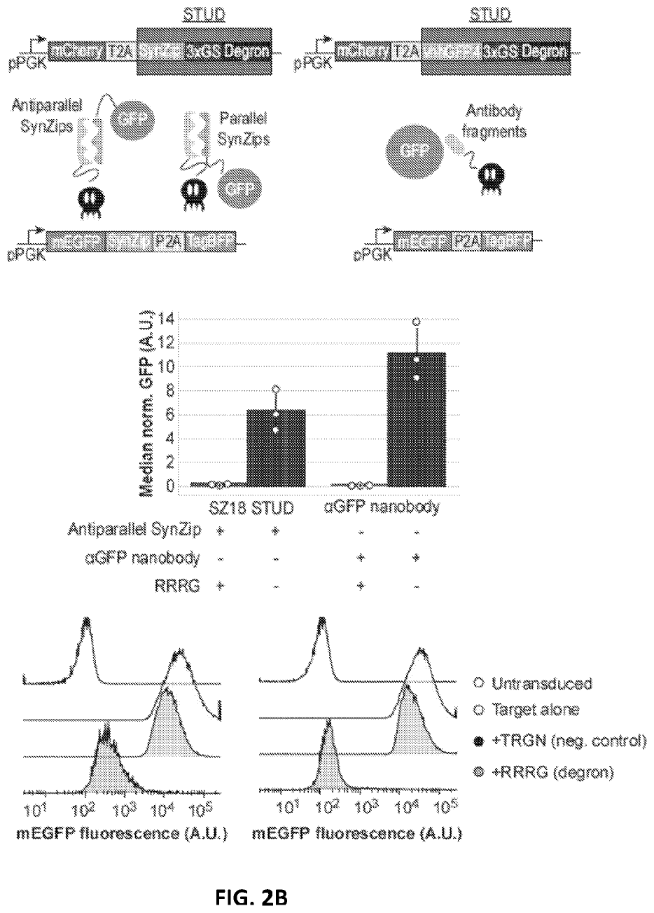

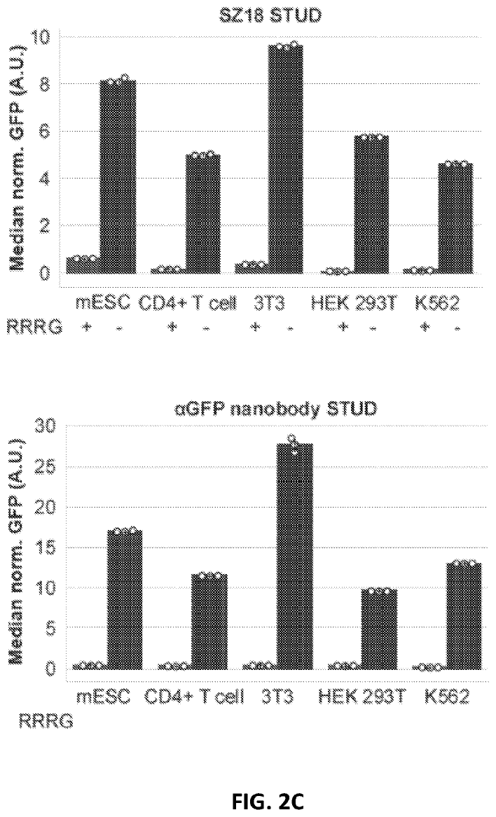

A new synthetic degradation molecule, referred to as a Synthetic Targeter of Ubiquitination and Degradation (‘STUD’), was designed. A STUD is composed of a binding domain that specifically identifies and dimerizes with target molecules and a degradation domain to recruit the UPP machinery to induce ubiquitination and subsequent degradation by the proteasome. Through these domains, STUDs act as a molecular bridge between the ubiquitin conjugation machinery of the UPP and a specific protein target of interest (FIG. 2A). Here STUD modularity is demonstrated with a minimal toolbox that consists of two orthogonal binding domains and two degradation domains. To emphasize the potential compactness of this system, previously described heterodimeric synthetic leucine zipper proteins were used (see Thompson et al supra 2012). It has also been demonstrated that STUDs can target proteins with minimal changes to their endogenous sequence using a nanobody that binds green fluorescent protein (GFP) (Saerens J. Mol. Bio. 2005 352:597-607). The degradation domain of choice for the STUDs shown in this work is a minimally sufficient sequence (‘+RRRG’; SEQ ID NO:32) from the FKBP degron that was previously described (Bonger 2011, supra). From this same work, a similar sequence with minimal observed degradation as a non-functional control was identified, which is referred to as ‘+TRGN’ (SEQ ID NO:50) or ‘−RRRG’ (SEQ ID NO:32) interchangeably.

Either the SynZip STUD and the antiGFP nanobody STUD in Jurkat T cells alongside a plasmid encoding either GFP fused to a complementary SynZip or GFP alone, respectively (FIG. 2B) were lentivirally transduced. 72 hours following removal of lentivirus, we assay for GFP fluorescence by flow cytometry. To quantify STUD degradation efficacy, we isolate cells by gating out cells with fluorescence values for both cotransduction markers less than those of untransduced (‘UnT’) Jurkat T cells. Normalized GFP fluorescence was calculated by normalizing individual cell GFP fluorescence by tagBFP fluorescence to account for any differences due to variations in plasmid expression or integration copy number. Looking at the median of the distributions of this normalized GFP for each condition, an approximately 42-fold change with the SynZip STUD and an approximately 167-fold change with the nanobody STUD was observed. Representative histograms of unnormalized GFP fluorescence are also shown for reference. FIG. 2C.

The potential wider application of STUDs as a tool for mammalian synthetic biology was demonstrated by replicating potent GFP degradation in other cell lines. For adherent cell lines (human embryonic (HEK) 293T, 3T3, and mouse embryonic stem cells (mESCs)), cells were seeded 24 hours before lentiviral transduction. While for suspension cell lines (K562 myelogenous leukemia cells and primary human CD4+ T cells) are plated the same day as a lentiviral addition. Experimental design following lentiviral addition is the same as for Jurkat T cells. Using the same analysis method as described above, it was observed that the degradation capability of STUDs is similarly efficacious across all tested cell lines.

In this example, the sequence of the STUD is shown below, where the underlined sequence is an alpha-helical heterodimerization domain (a modified synZIP), the heptad leucines are in large letters, the optional flexible linker is in bold and the degron is in italics.

synZIP STUD:
(SEQ ID NO: 413)
SIAATLENDLARLENENARLERDIANLERDLARLEREEAYFGSGSGSG
SGSRRRG

The heterodimerization domain of this STUD is the same as synZIP18 described by Thompson et al (ACS Synth Biol. 2012 1: 118-129) except that the lysine residues have been substituted with arginine residues.

The sequence of synZIP18 is shown below, with the lysine residues underlined.

synZIP18:
(SEQ ID NO: 18)
SIAATLENDLARLENENARLEKDIANLERDLAKLEREEAYF

The binding partner for this STUD is shown below, where the heptad leucines are in large underlined letters.

Binding partner for STUD
(SEQ ID NO: 17)
NEKEELKSKKAELRNRIEQLKQKREQLKOKIANLRKEIEAYK

This sequence is synZIP18, as described by Thompson et al (ACS Synth Biol. 2012 1: 118-129). Note that the lysines do not need to be removed from this sequence because this sequence is in the target protein.

### Example 2

### Loss in Target Signal can be Rescued by Inhibition of UPP

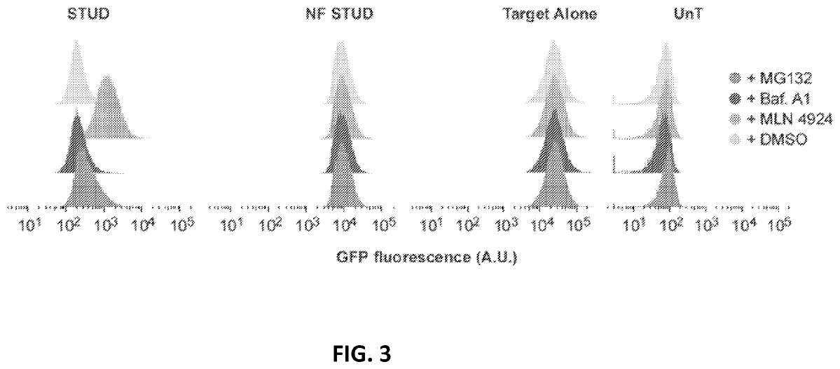

Inhibitors of the proteasomal and lysosomal degradation pathways were used to demonstrate that loss in GFP fluorescence can be attributed to degradation. Using the 2 plasmid system as described above, we lentivirally transduce Jurkat T cells. 72 hours after removal of lentivirus, we treat these Jurkat T cells and an untransduced control cell line with either 5 μM of the proteasomal inhibitor MG-132, 1 μM of the cullin ring ligase inhibitor MLN4924, 100 nM of the lysosomal inhibitor Bafilomycin A1, or DMSO vehicle control and incubate at 37 C for 5 hours. Using flow cytometry to measure GFP fluorescence following treatment, we observe that GFP fluorescence can indeed be rescued with MG-132 and MLN4924 when cells express both a functional STUD and a GFP target relative to DMSO vehicle control. No changes to GFP fluorescence were seen with bafilomycin treatment. Similar GFP fluorescence values were observed in cells expressing either GFP target and a non-functional STUD or GFP alone across all conditions. Together, these data show that loss of GFP fluorescence in the presence of a STUD is due to degradation and that this degradation is mediated by the proteasome. FIG. 3.

### Example 3

### Tethering of STUD to Plasma Membrane Allows for Functional Knockdown for Second-Generation Chimeric Antigen Receptors

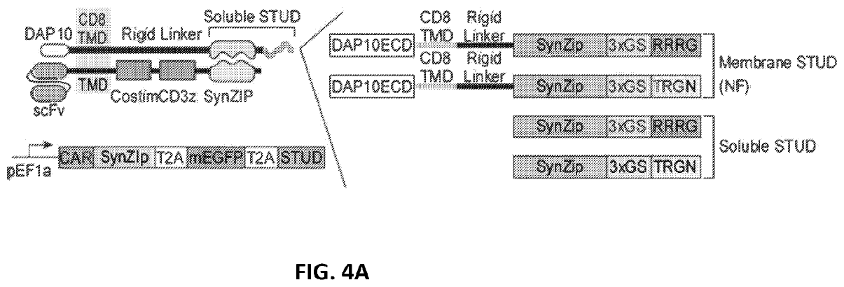

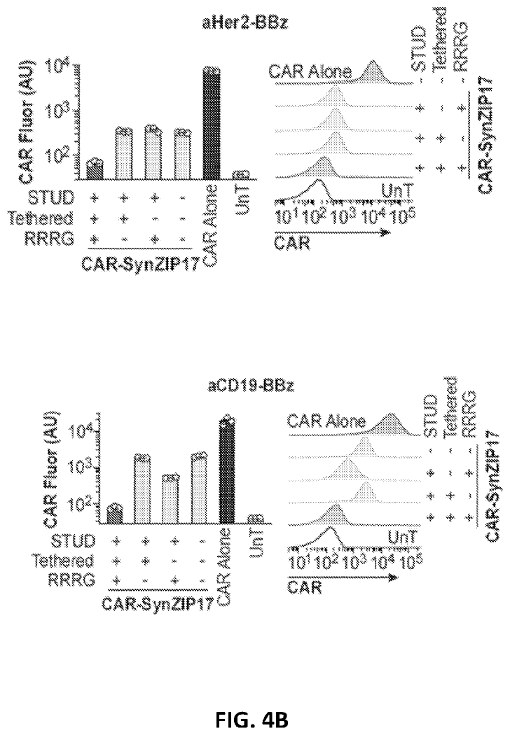

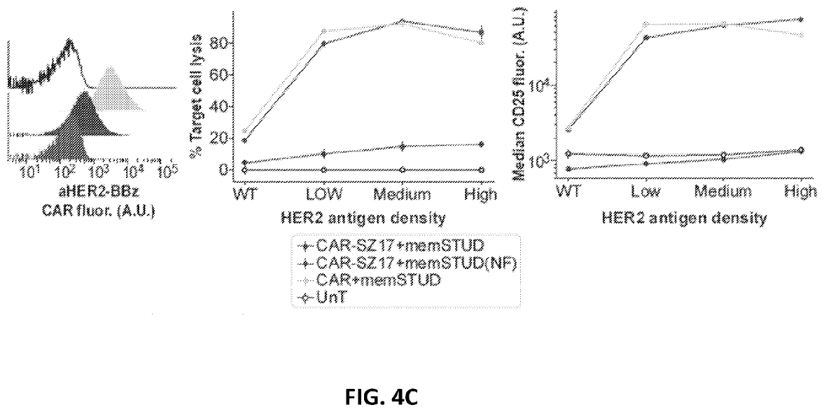

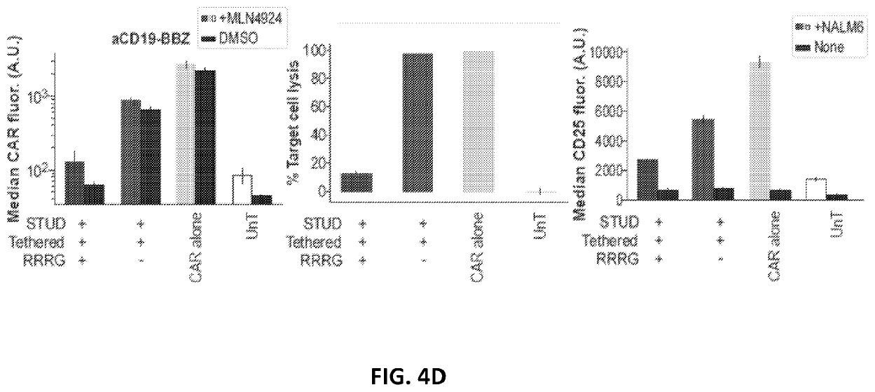

In initial tests, it was found that a STUD alone only degraded membrane proteins, namely a chimeric antigen receptor (CAR), inefficiently. Increasing the local concentration of the STUD at the membrane by fusion to a membrane localization domain was tested. The STUD was fused to a previously published membrane localization domain consisting of a truncated extracellular domain from the DAP10 protein and a transmembrane domain from the CD8 alpha protein (Wu 2015). The plasmids were lentivirally transduced into cells encoding this new membrane-tethered STUD (‘memSTUD’), a variant of the memSTUD with the non-functional sequence used in previous figures, or the original version of the STUD described in previous figures (‘soluble STUD’) along with a second-generation CAR and a GFP transduction marker (FIG. 4A). The ability of the memSTUDs to degrade two 4-1BB variant second-generation (‘BBz’) CARs that target CD19 or HER2 in Jurkat T cells was tested. 72 hours after removal of lentivirus, an antibody stain specific for an extracellular myc tag fused to the CAR was used. The surface CAR expression by fluorescence of this antibody stain was measured by flow cytometry. It was observed that memSTUDs are able to potently degrade both types of 4-1BB CARs (FIG. 4B).

Next, CD8+ primary human T cells were lentivirally transduced with the same constructs as described above, isolated populations of interest by FACS, and co-cultured these cell populations with target cells expressing either a CAR antigen or no antigen for 72 hours. For HER2BBz CARs, we coculture engineered CD8+ T cells with K562 target cells expressing variable levels of HER2 antigen (Hernandez-Lopez 2021). Target cell lysis and expression of the T cell activation marker CD25 were measured after 72 hours of coculture (FIG. 4C).

Using the CD19BBz CAR, it was first demonstrate that incubation of 1 μM of MLN4924 for 5 hours at 37 C can rescue STUD knockdown of CAR expression (FIG. 4D). Engineered CD8+ T cells expressing were cocultured with NALM6 target cells and measured activation of the CAR by quantifying target cell lysis and expression of the T cell activation marker CD25. From these experiments, greatly diminished cell lysis and expression of CD25 from cells expressing the memSTUD relative to cells expressing a CAR alone or the non-functional STUD (FIG. 4C&D) was observed. From these data, it was concluded that membrane tethering of a STUD is necessary for sufficient knockdown of CAR proteins and that this knockdown is able to functionally disable the CAR.

### Example 4

### Design of New Synthetic Receptor Allows for Antigen Triggered Degradation of Cytosolic Proteins

Synthetic Notch receptors, and the newly published Synthetic Intramembrane Proteolysis Receptors (SNIPRs), are a class of synthetic proteins that borrow from the Notch family of receptors (Morsut, et al Cell 1016 164: 780-791, Zhu et al bioRxiv, posted May 23, 2021. Roybal, et al Cell 2016 167, 419-432) These molecules have a customizable intracellular transcription factor that gets released from the membrane upon antigen recognition and binding. It was hypothesized that we could exchange the transcription factor for a STUD to result in antigen-dependent degradation of a cytosolic target. By combining the extracellular antigen recognition domain and the transmembrane and juxtamembrane domains from SNIPRs, a novel proteolytic receptor, the ‘NotchSTUD’, was designed.

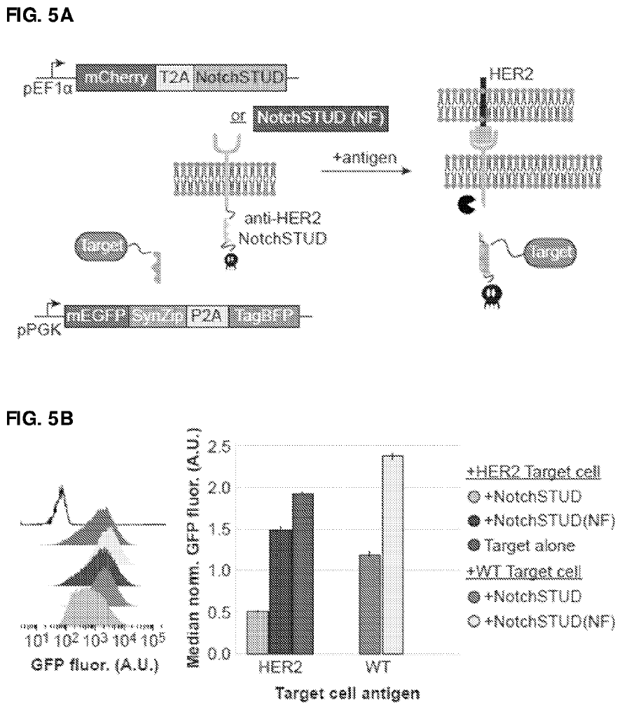

Here, the NotchSTUD was used to degrade a GFP-SynZip target in an antigen-dependent manner. CD4+ primary human T cells were lentivirally induced with a two plasmid system. The first encodes the NotchSTUD and mCherry cotransduction marker and the second encodes the same GFP target described in FIG. 2. Following lentiviral transduction, cells expressing both of these plasmids by FACS were isolated and the cells were cocultured with K562 target cells expressing either the NotchSTUD antigen (HER2) or wild-type cells that express no antigen (FIG. 5A). After 72 hours of culture, we measure GFP fluorescence by flow cytometry. GFP fluorescence was quantified by normalizing using the same method as described above. From these assays, we observe modest decrease in the median normalized GFP fluorescence in cells that express the NotchSTUD co-cultured with HER2 target cells relative to the same cells cocultured with WT target cells (FIG. 5B). Minimal change in normalized GFP fluorescence was observed in cells that express a nonfunctional NotchSTUD.

### Example 5

### STUDs Can be Composed into Negative Feedback Circuit to Regulate Synthetic Transcription Factor (SynTF)

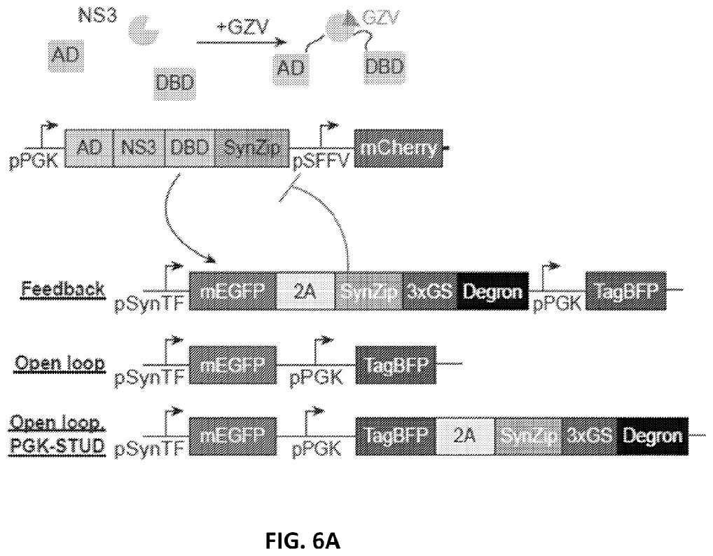

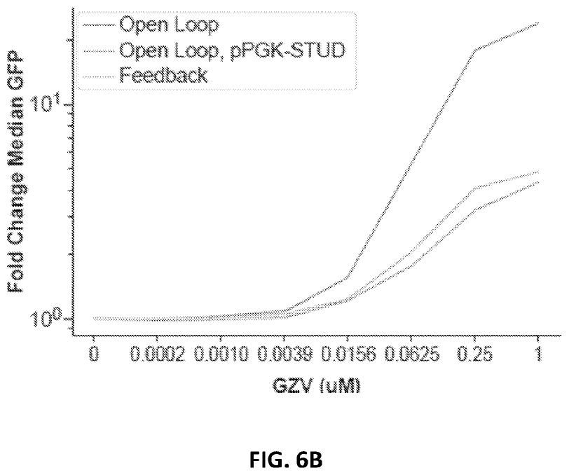

It is shown that STUDs can be composed into molecular circuits by demonstrating the use of STUDs in a negative feedback loop. The circuit has three components: (1) a synthetic drug-inducible transcription factor (SynTF) fused to a SynZip, (2) a GFP reporter, and (3) a SynZip STUD that targets the SynTF (FIG. 6A). The SynTF relies on previously published NS3 protease from the Hepatitis C virus and the small-molecule drug grazoprevir (GZV) (cite). GZV is an inhibitor of the NS3 protease and in the absence of GZV, the protease is active and the SynTF is non-functional. On the other hand, in the presence of GZV, the NS3 is inhibited and the SynTF is stable and functional. A stable SynTF then drives the production of the GFP reporter and the STUD feedback cassette. We also built two open loop control circuits. The first has SynTF drive the GFP reporter alone while the second has a constitutively expressed STUD that continuously degrades the SynTF. We introduce component 1 and components 2 and 3 as a two plasmid system into Jurkat T cells by lentivirally transduction. 72 hours after removal of lentivirus, we induce these cells with GZV for 72 hours at 37 C. After incubation, we assay for GFP fluorescence by flow cytometry and gate on cells expressing the co-transduction markers (mCherry and tagBFP) for both plasmids relative to a untransduced control. Looking at the fold change of the median GFP at each concentration of drug relative to a DMSO vehicle control, we find that the STUD negative feedback circuit inhibits the SynTF closely resembling the inhibition of the constitutive STUD open loop control (FIG. 6B). From these data, it was concluded that the STUD can be incorporated into negative feedback loops and powerfully regulate synTFs.

### Example 6

### Dose Dependent Degradation of STUDs Allow for Use as ON Switch CAR

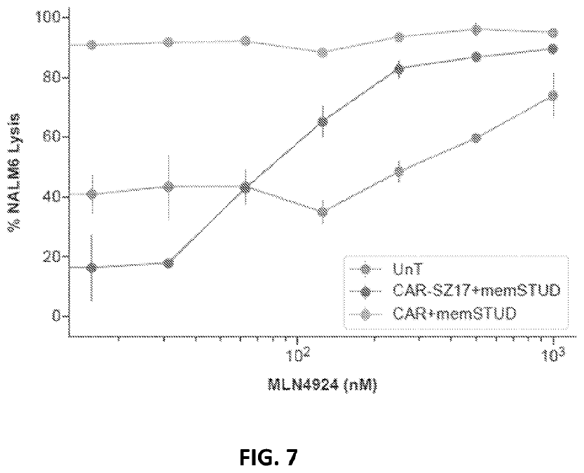

CD8+ primary human T cells were engineered with an antiCD19BBz CAR fused to a SynZip and a memSTUD that binds the SynZip using the same plasmids outlined in FIG. 14A by lentiviral transduction. As a control, we also lentivirally transduce CD8+ primary human T cells with an antiCD19BBz and a SynZip memSTUD which cannot bind the CAR. These engineered cells and an untransduced control were cocultured with NALM6 target cells for 72 hours at 37 C in the presence of MLN4924 ranging from 1 μM to 0.015625 μM and a DMSO vehicle control. After coculture, target cell lysis was assayed by flow cytometry. Specific target cell lysis of each cell line relative to NALM6 cells cultured alone in the same conditions was then calculated. It was found that that the activity of the STUD can be titrated such that lysis by CAR is dose dependent which can be used as an ON switch CAR in future applications (FIG. 7).

### Materials and Methods

Cytosolic STUD for targeting GFP: Cytosolic STUDs were introduced by lentiviral transduction of two plasmids. The first encodes a green fluorescent protein (GFP) which will be a target for degradation alongside a BFP as a co-transduction marker. The second encodes the STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of GFP fluorescence.

Using proteasome inhibitor to explore cytosolic GFP mechanism: To ascertain the mechanism by which the STUD degrades cytosolic GFP, we incubated cells with 5 μM of the proteasome inhibitor MG132 for 1 and 3 hours. Cells were then washed with PBS and analyzed by flow cytometry. Using the same 2-plasmid system as described above, we measured changes in GFP fluorescence relative to controls.

Membrane targeting STUD: Membrane targeting cells were introduced by lentiviral transduction of two plasmids. The first encodes a chimeric antigen receptor (CAR) or synthetic Notch (SynNotch) protein which will be a target for degradation alongside a BFP as a co-transduction marker. The second encodes the membrane localized STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of CAR/SynNotch. CAR and SynNotch expression was measured by antibody staining for a peptide tag fused to the extracellular domain of the CAR/SynNotch.

Cell culture for Lenti-X 293T cells: Lenti-X 293T packaging cells (Clontech #11131D) were cultured in medium consisting of Dulbecco's Modified Eagle Medium (DMEM) (Gibco #10569-010) and 10% fetal bovine serum (FBS) (University of California, San Francisco [UCSF] Cell Culture Facility). Lenti-X 293T cells were cultured in T150 or T225 flasks (Corning #430825 and #431082) and passaged every 2-3 days upon reaching 70-80% confluency. To passage, cells were treated with TrypLE express (Gibco #12605010) at 37 C for 5 minutes. Then, 10 mL of media was used to quench the reaction and cells were collected into a 50 mL conical tube and pelleted by centrifugation (400×g for 4 minutes). Cells were cultured until passage 30 whereupon fresh Lenti-X 293 T cells were thawed.

Cell culture for HEK 293T cells: HEK 293T cells (UCSF Cell Culture Facility) were cultured in medium consisting of Dulbecco's Modified Eagle Medium (DMEM) (Gibco #10569-010) and 10% fetal bovine serum (FBS) (UCSF Cell Culture Facility). HEK 293T cells were cultured in T75 flasks (Corning #430641U) and passaged every 2-3 days upon reaching 70-80% confluency.

Cell culture for 3T3 cells: 3T3 cells were cultured in medium consisting of Dulbecco's Modified Eagle Medium (DMEM) (Gibco #10569-010) and 10% fetal bovine serum (FBS) (UCSF Cell Culture Facility). 3T3 cells were passaged upon reaching 70-80% confluency. To pass, cells were treated with TrypLE express at 37 C for 3 minutes. Then, 10 mL of media was added to quench the reaction and cells were collected into a 50 ml conical tube and pelleted by centrifugation (400×g for 4 minutes). Pellet was resuspended in 5 mL and 1 mL of resuspended pellet was added to a T25 flask (Corning #430639) containing 10 mL of media.

Cell culture for Jurkat T cells: Jurkat T cells (UCSF Cell Culture Facility) were cultured in media consisting of RPMI-1640 (ThermoFisher Scientific #11875093), 10% FBS (UCSF Cell Culture Facility) and 1% antibiotics-antimycotics (ThermoFisher Scientific #15240062). To passage, cells were maintained at a concentration of 1×10\hat{ }6 cells/mL in a T150 flask. Cells were cultured until passage 30 whereupon fresh Jurkat T cells were thawed.

Cell culture for K562 myelogenous leukemia cells: K562 cells were cultured in media consisting of Iscove's Modified Dulbecco's Medium (ThermoFisher Scientific #12440053), 10% FBS (UCSF Cell Culture Facility) and 1% Gentamicin (ThermoFisher Scientific #15750078). To passage, cells were maintained at a concentration of 1×10\hat{ }6 cells/mL in a T25 flask.

Culture of mouse embryonic stem cells (mESCs): mESCs were cultured in “Serum Free ES” (SFES) media supplemented with 2i. SFES media consists of 500 mL DMEM/F12 (Gibco #11320-033), 500 mL Neurobasal (Gibco #21103-049), 5 mL N2 Supplement (Gibco #17502-048), 10 mL B27 with retinoic acid (gibco #17504-044), 6.66 mL 7.5% BSA (Gibco #15260-037), 10 mL 100× GlutaMax (Gibco #35050-061), and 10 mL 100× Pen/Strep. To make “2i SFES”, 1 nM PD03259010 (Selleckchem #S1036), 3 nM CHIR99021 (Selleckchem #S2924) and 1000 units/mL LIF (ESGRO #ESG1106) were added to 45 mL SFES. Prior to use, 1-thioglycerol (MTG; Sigma M6145) was diluted 1.26% in SFES and added 1:1000 to 2i SFES media. To passage, mESCs were treated with 1 mL of accutase in a 6 well plate (Corning #353046) for 5 minutes at room temperature (RT). After incubation, cells were mixed by pipette and moved to a 15 mL conical tube, supplemented with 10 mL SFES and spun at 300×g for 3 minutes. Then, media was removed and cells were counted using the Countess II Cell Counter (ThermoFisher) according to the manufacturer's instructions. Cells were then plated in 6 well plates that had gelatinized with 1% gelatin for 30 minutes at 37 C at 5×10\hat{ }5 cells per well in 2 mL of 2i SFES. Media was changed every day and cells were split every other day.

Primary Human T Cell Isolation and Culture: Primary CD4+ and CD8+ T cells were isolated from anonymous donor blood after apheresis by negative selection (STEMCELL Technologies #15062 and 15023). T cells were cryopreserved in RPMI-1640 (Corning #10-040-CV) with 20% human AB serum (Valley Biomedical, #HP1022) and 5% DMSO (Sigma-Aldrich #472301). After thawing, T cells were cultured in human T cell medium (hTCM) consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

Lentiviral transduction of primary T cells: Pantropic VSV-G pseudotyped lentivirus was produced via transfection of Lenti-X 293T cells with a modified pHR′SIN: CSW transgene expression vector and the viral packaging plasmids pCMVAR8.91 and pMD2.G using Fugene HD (Promega #E2312). Primary T cells were thawed the same day, and after 24 hr in culture, were stimulated with Dynabeads Human T-Activator CD3/CD28 (Thermo Scientific #11131D) at a 1:3 cell:bead ratio. At 48 hr, viral supernatant was harvested and concentrated using the Lenti-X concentrator (Takara, #631231) according to the manufacturer's instructions. Briefly, viral supernatant was harvested and potential contaminants were filtered using a 0.45 μM filter (Millipore Sigma #SLHV033RS). Lenti-X concentrator solution was added at a 1:3 viral supernatant: concentrator ratio, mixed by inversion, and incubated at 4 C for at least 2 hours. Supernatant-concentrator mix was pelleted by centrifugation at 1500×g at 4 C for 45 minutes, supernatant was removed and pellet was resuspended using 100 μL media or PBS (UCSF Cell Culture Facility) for each well of T cells. Typically, 2 wells of a 6 well plate was concentrated for 1 well of a 24 well plate plated with 1 million T cells on day of transfection. The primary T cells were exposed to the virus for 24 hr and viral supernatant was exchanged for fresh hTCM supplemented with IL-2 as described above. At day 5 post T cell stimulation, Dynabeads were removed and the T cells expanded until day 12-14 when they were rested for use in assays. For co-culture assays, T cells were sorted using a Sony SH-800 cell sorter on day 5-6 post stimulation.

Construct assembly: All plasmids were constructed using a previously described hierarchical DNA assembly method based on Golden Gate cloning (Lee 2015, Fonseca 2019). Plasmids were verified by sequencing and/or restriction digest and gel electrophoresis.

Flow cytometry: All flow cytometry data was obtained using a LSR Fortessa (BD Biosciences). All assays were run in a 96-well round bottom plate (Fisher Scientific #08-772-2C). Samples were prepared by pelleting cells in the plate using centrifugation at 400×g for 4 minutes. Supernatant was then removed and 200 μL of PBS (UCSF Cell Culture facility) was used to wash cells. The cells were again pelleted as described above and supernatant was removed. Cells were resuspended in 120 μL of Flow buffer (PBS+2% FBS) and mixed by pipetting prior to flow cytometry assay.

Inhibitor Assays: 100,000 cells were plated in a 96 well round bottom plate with either 5 μM MG-132 (Sigma-Aldrich #M7449-200UL), 1 μM MLN4924 (Active Biochem #A-1139), 100 nM Bafilomycin A1 (Enzo Life Sciences #BML-CM110-0100), or DMSO vehicle control and incubated at 37 C for 5 hours. After incubation, cells were pelleted by centrifugation at 400×g for 4 minutes. Supernatant was then removed and cells were washed once with 200 μL PBS. Cells were pelleted again (400×g for 4 minutes) and resuspended in flow buffer (PBS+2% FBS) for assay by flow cytometry.

Antibody staining: All experiments using antibody staining were performed in 96 well round bottom plates. Cells for these assays were pelleted by centrifugation (400×g for 4 minutes) and supernatant was removed. Cells were washed once with 200 μL of PBS and pelleted again by centrifugation (400×g for 4 minutes) and the supernatant was removed. Cells were resuspended in a staining solution of 50 μL PBS containing fluorescent antibody stains of interest. Anti-myc antibodies (Cell Signaling Technologies #2233S and #2279S) was used at a 1:100 ratio while antiV5 (ThermoFisher Scientific #12-679642) and antiFLAG (R&D Systems #IC8529G-100) antibodies were used at a 1:50 ratio for flow cytometry assays. For FACS, all antibodies were used in a 1:50 ratio in 100 μL.

Generation of coculture target cells: HER2-expressing K562 target cells were previously characterized in the literature and were a gift from Dr. Wendell Lim (Hernandez-Lopez 2021). CD19-expressing K562 cells were generated by lentiviral transduction and antibiotic selection with 2 μg/mL puromycin for one week.

NALM6 cell culture: NALM6 cells were cultured in medium consisting of RPMI-1640, 10% fetal bovine serum (FBS) (University of California, San Francisco [UCSF] Cell Culture Facility), and 1% antibiotics-antimycotics. To passage, cells were maintained at a concentration of 1×10\hat{ }6 cells/mL in a T25 flask.

Co-culture assays: For all assays, T cells and target cells were co-cultured at a 1:1 ratio with cell numbers varying per assay. All assays contained between 10,000 and 50,000 of each cell type. The Countess II Cell Counter (ThermoFisher) was used to determine cell counts for all assays set up. T cells and target cells were mixed in 96-well round bottom tissue culture plates in 200 μL T cell media, and then plates were centrifuged for 1 min at 400×g to initiate interaction of the cells prior to incubation at 37 C.

Data analysis: Data analysis was performed using the FlowJo software (FlowJo LLC.) and Python. For co-culture assays, desired cell populations were isolated by FACS using a Sony SH800 cell sorter. For non co-culture assays, desired cell populations were isolated by gating in FlowJo following flow cytometry.

Grazoprevir (GZV) induction: 25,000 Jurkat T cells were seeded into 96 well round bottom plates in 100 μL fresh media. 100 μL containing media containing a 2× concentration of GZV was added to each well of seeded cells. Cells with GZV were incubated at 37 C for 72 hours. DMSO vehicle at the same concentration as the max GZV concentration was added to cells as a control.

MLN dose response: 25,000 CD8+ primary human T cells were seeded into 96 well round bottom plates in 100 μL fresh media. 100 μL containing media containing a 2× concentration of MLN4924 was added to each well of seeded cells. Cells with MLN4924 were incubated at 37 C for 72 hours. DMSO vehicle at the same concentration as the max MLN4924 concentration was added to cells as a control.

### REFERENCES

1. Bonger, K. M., Chen, L.-C., Liu, C. W. & Wandless, T. J. Small-molecule displacement of a cryptic degron causes conditional protein degradation. Nat. Chem. Biol. 7, 531-537 (2011).
2. Daniel, K. et al. Conditional control of fluorescent protein degradation by an auxin-dependent nanobody. Nat. Commun. 9, 3297 (2018).
3. Arai, R., Ueda, H., Kitayama, A., Kamiya, N. & Nagamune, T. Design of the linkers which effectively separate domains of a bifunctional fusion protein. Protein Eng. 14, 529-532 (2001).
4. Wu, C. Y., Roybal, K. T., Puchner, E. M. & Onuffer, J. Remote control of therapeutic T cells through a small molecule-gated chimeric receptor. Science (2015).

What is claimed is:

1. A fusion protein for degrading a target protein in trans, comprising:
a lysine-free alpha-helical heterodimerization domain; and
a C-terminal degron.

2. The fusion protein of claim 1, wherein the heterodimerization domain is 30-80 aa in length.

3. The fusion protein of claim 1 or 2, wherein the fusion protein is no more than 100 aa in length.

4. The fusion protein of any one of claims 1-3, wherein the fusion protein is 40-100 amino acids in length.

5. The fusion protein of any one of claims 1-4, wherein the heterodimerization domain comprises a bZIP domain.

6. The fusion protein of any one of claims 1-5, wherein the C-terminal degron is RRRGN (SEQ ID NO:375) or RRRG (SEQ ID NO:32).

7. The fusion protein of any one of claims 1-6, wherein the heterodimerization domain comprises a sequence that is at least 80% identical to at least 35 contiguous amino acids of any of any sequence in table 1, wherein any lysine residues are substituted.

8. The fusion protein of claim 7, wherein any lysine residues are substituted with an arginine residue.

9. The fusion protein of any one of claims 1-8, further comprising a flexible linker between the heterodimerization domain and the C-terminal degron.

10. The fusion protein of claim 9, wherein the flexible linker is 2-20 amino acids in length.

11. The fusion protein of any one of claims 1-10, further comprising a transmembrane domain that is N-terminal to the heterodimerization domain.

12. The fusion protein of any one of claims 1-11, wherein the fusion partner comprises amino acids 1-41 or all of SEQ ID NO: 413.

13. A recombinant nucleic acid comprising:
a promoter and a coding sequence for the fusion protein of any one of claims 1-12,
wherein promoter and coding sequence are operably linked.

14. The nucleic acid of claim 13, wherein the promoter is chemically-inducible, tissue-specific, cell state-responsive, or activated by an external stimulus that is recognized at the plasma membrane.

15. A cell comprising the nucleic acid of claim 13 or 14.

16. The cell of claim 15, wherein the cell is eukaryotic.

17. The cell of claim 15 or 16, wherein the cell is a therapeutic cell.

18. The cell of any one of claims 15-17, wherein nucleic acid is integrated into the genome of the cell or exogenous to the genome of the cell.

19. The cell of any one of claims 15-18, further comprising a target protein that comprise a binding partner for the lysine-free alpha-helical heterodimerization domain.

20. The cell of claim 19, wherein the target protein is a recombinant transcription factor, enzyme, kinase, receptor or cytokine.

21. The cell of claim 19 or 20, wherein the target protein comprises the amino acid sequence of SEQ ID NO: 17.

22. A method for degrading a target protein comprising:
inducing expression of the fusion protein in a cell of any one of claims 15-21.

23. A method of treatment comprising:
administering a cell of any of claims 15-21 to an individual in need thereof.


SEQUENCE LISTING (413 sequences)

SEQ ID NO: 1 - protein, 47 aa, synthetic construct
NLVAQLENEVASLENENETLKKKNLHKKDLIAYLEKEIANLRKKIEE

SEQ ID NO: 2 - protein, 50 aa, synthetic construct
ARNAYLRKKIARLKKDNLQLERDEQNLEKIIANLRDEIARLENEVASHEQ

SEQ ID NO: 3 - protein, 42 aa, synthetic construct
NEVTTLENDAAFIENENAYLEKEIARLRKEKAALRNRLAHKK

SEQ ID NO: 4 - protein, 54 aa, synthetic construct
QKVAELKNRVAVKLNRNEQLKNKVEELKNRNAYLKNELATLENEVARLENDVAE

SEQ ID NO: 5 - protein, 43 aa, synthetic construct
TVKELKNYIQELEERNAELKNLKEHLKFAKAELEFELAAHKFE

SEQ ID NO: 6 - protein, 54 aa, synthetic construct
QKVAQLKNRVAYKLKENAKLENIVARLENDNANLEKDIANLEKDIANLERDVAR

SEQ ID NO: 7 - protein, 51 aa, synthetic construct
KEIEYLEKEIERLKDLREHLKQDNAAHRQELNALRLEEAKLEFILAHLLST

SEQ ID NO: 8 - protein, 51 aa, synthetic construct
KEIANLEKEIASLEKKVAVLKQRNAAHKQEVAALRKEIAYVEDEIQYVEDE

SEQ ID NO: 9 - protein, 36 aa, synthetic construct
QKVESLKQKIEELKQRKAQLKNDIANLEKEIAYAET

SEQ ID NO: 10 - protein, 42 aa, synthetic construct
NLLATLRSTAAVLENENHVLEKEKEKLRKEKEQLLNKLEAYK

SEQ ID NO: 11 - protein, 42 aa, synthetic construct
ELTDELKNKKEALRKDNAALLNELASLENEIANLEKEIAYFK

SEQ ID NO: 12 - protein, 42 aa, synthetic construct
NEDLVLENRLAALRNENAALENDLARLEKEIAYLEKEIEREK

SEQ ID NO: 13 - protein, 44 aa, synthetic construct
QKVEELKNKIAELENRNAVKKNRVAHLKQEIAYLKDELAAHEFE

SEQ ID NO: 14 - protein, 47 aa, synthetic construct
NDLDAYEREAEKLEKKNEVLRNRLAALENELATLRQEVASMKQELQS

SEQ ID NO: 15 - protein, 41 aa, synthetic construct
FENVTHEFILATLENENAKLRRLEAKLERELARLRNEVAWL

SEQ ID NO: 16 - protein, 42 aa, synthetic construct
NILASLENKKEELKKLNAHLLKEIENLEKEIANLEKEIAYFK

SEQ ID NO: 17 - protein, 42 aa, synthetic construct
NEKEELKSKKAELRNRIEQLKQKREQLKQKIANLRKEIEAYK

SEQ ID NO: 18 - protein, 41 aa, synthetic construct
SIAATLENDLARLENENARLEKDIANLERDLAKLEREEAYF

SEQ ID NO: 19 - protein, 45 aa, synthetic construct
NELESLENKKEELKNRNEELKQKREQLKQKLAALRNKLDAYKNRL

SEQ ID NO: 20 - protein, 44 aa, synthetic construct
STVEELLRAIQELEKRNAELKNRKEELKNLVAHLRQELAAHKYE

SEQ ID NO: 21 - protein, 42 aa, synthetic construct
NEVAQLENDVAVIENENAYLEKEIARLRKEIAALRDRLAHKK

SEQ ID NO: 22 - protein, 50 aa, synthetic construct
KRIAYLRKKIAALKKDNANLEKDIANLENEIERLIKEIKTLENEVASHEQ

SEQ ID NO: 23 - protein, 36 aa, synthetic construct
ERIARLEEKVKTLKAQNSELASTANMLREQVAQLKQ

SEQ ID NO: 24 - protein, 36 aa, synthetic construct
ERIARLEDKVKTLKAENAGLSSTAGLLREQVAQLKQ

SEQ ID NO: 25 - protein, 36 aa, synthetic construct
ELTDTLQAETDQLEDEKSALQTEIANLLKEKEKLEF

SEQ ID NO: 26 - protein, 36 aa, synthetic construct
EKTECLQKESEKLESVNAELKAQIEELKNEKQHLIY

SEQ ID NO: 27 - protein, 36 aa, synthetic construct
AEQEALTGECKELEKKNEALKERADSLAKEIQYLKD

SEQ ID NO: 28 - protein, 36 aa, synthetic construct
AEGEALEGECQGLEARNRELKERAESVEREIQYVKD

SEQ ID NO: 29 - protein, 43 aa, synthetic construct
QRNVETQQKVLELTSDNDRLRKRVEQLSRELDTLRGIFRQLPE

SEQ ID NO: 30 - protein, 43 aa, synthetic construct
MRNLETQHKVLELTAENERLQKKVEQLSRELSTLRNLFKQLPE

SEQ ID NO: 31 - protein, 43 aa, synthetic construct
QKAQDTLQRVNQLKEENERLEAKIKLLTKELSVLKDLFLEHAH

SEQ ID NO: 32 - protein, 4 aa, synthetic construct
RRRG

SEQ ID NO: 33 - protein, 43 aa, synthetic construct
RRILETQQKVLEYMAENERLRSRVEQLTQELDTLRNLFRQIPE

SEQ ID NO: 34 - protein, 22 aa, synthetic construct
RKVAQLAEENERLKQEIERLTR

SEQ ID NO: 35 - protein, 29 aa, synthetic construct
EYVKCLENRVAVLENQNKTLIEELKALKD

SEQ ID NO: 36 - protein, 29 aa, synthetic construct
LNDLVLENKLIALGEENATLKAELLSLKL

SEQ ID NO: 37 - protein, 29 aa, synthetic construct
QKADTLHLESEDLEKQNAALRKEIKQLTE

SEQ ID NO: 38 - protein, 29 aa, synthetic construct
DKADALHQQHESLEKDNLALRKEIQSLQA

SEQ ID NO: 39 - protein, 36 aa, synthetic construct
QKADKLHEEYESLEQENTMLRREIGKLTEELKHLTE

SEQ ID NO: 40 - protein, 34 aa, synthetic construct
RDARRLKENQIAIRASFLEKENSALRQEVADLRK

SEQ ID NO: 41 - protein, 29 aa, synthetic construct
QQKHHLENEKTQLIQQVEQLKQEVSRLAR

SEQ ID NO: 42 - protein, 64 aa, synthetic construct
GSPKEEARELIRKQKELIKEQKKLIKEAKQKSDSRDAERIWKRSREINRESKKINKRIKELIKS

SEQ ID NO: 43 - protein, 62 aa, synthetic construct
PKKEAEELAEESEELHDRSEKLHERAEQSSNSEEARKILEDIERISERIEEISDRIERLLRS

SEQ ID NO: 44 - protein, 77 aa, synthetic construct
GTKEDILERQRKIIERAQEIHRRQQEILEELERIIRKPGSSEEAMKRMLKLLEESLRLLKELLELSEESAQLLYEQR

SEQ ID NO: 45 - protein, 83 aa, synthetic construct
GTEKRLLEEAERAHREQKEIIKKAQELHRRLEEIVRQSGSSEEAKKEAKKILEEIRELSKRSLELLREILYLSQEQKGSLVPR

SEQ ID NO: 46 - protein, 76 aa, synthetic construct
TKEDILERQRKIIERAQEIIRRQQEILEELERIIRKPGSSEEAMKRMLKLLEESLRLLKELLELLEESAQLLYEQR

SEQ ID NO: 47 - protein, 78 aa, synthetic construct
GSTEKRLLEEAERAHREAKEIIKKAQELHRRLEEIVRQSGSSEEAKKEAKKILEEIRELSKRLLELLREILYLSQEQK

SEQ ID NO: 48 - protein, 76 aa, synthetic construct
TKEDILERARKIIERAQEIHRRQQEILEELERIIRKPGSSEEAMKRMLKLLEESLRLLKELLELSEELAQLLYEQR

SEQ ID NO: 49 - protein, 78 aa, synthetic construct
GSTEKRLLEEAERAIREQKEIIKKAQELHRRLEEIVRQSGSSEEAKKEAKKILEEIRELSKRSLELLREILYLLQEQK

SEQ ID NO: 50 - protein, 4 aa, synthetic construct
TRGN

SEQ ID NO: 51 - protein, 35 aa, synthetic construct
TKEDILERQRKIIERAQEIHRRQQEILEELEYIIR

SEQ ID NO: 52 - protein, 121 aa, synthetic construct
MSEEAMKRMLKLLEESLRLLKELLELSEESAQLLYEQRKANNGSETEKRLLEEAERAHREQKEIIKKAQELHRRLEEIVRQSGSSEEAKKEAKKILEEIRELSKRSLELLREILYLSQEQK

SEQ ID NO: 53 - protein, 77 aa, synthetic construct
MTKEDILERQRKIIERAQEIHRRQQEILKEQEKIIRKPGSSEEAMKRSLKLIEESLRLLKELLELSEESAQLLYEQR

SEQ ID NO: 54 - protein, 77 aa, synthetic construct
GTEKRLLEEAERAHREQKEIIKKAQELHKELTKIHQQSGSSEEAKKRALKISQEIRELSKRSLELLREILYLSQEQK

SEQ ID NO: 55 - protein, 76 aa, synthetic construct
TKEDILERQRKIIERAQEIHRRQQEILKRSEEIIRKPGSSEEALETLRELQEESLRLLKELLELSEESAQLLYEQR

SEQ ID NO: 56 - protein, 78 aa, synthetic construct
GSTEKRLLEEAERAHREQKEIIKKAQELHRRTEEIIRQSGSSEEAKDELRRIQEEIRELSKRSLELLREILYLSQEQK

SEQ ID NO: 57 - protein, 36 aa, synthetic construct
TTKRYLEEAERAHREQKEIIKKAQELHRRLEEIVRQ

SEQ ID NO: 58 - protein, 116 aa, synthetic construct
GSSEEAKKEAKKILEEIRELSKRSLELLREILYLSQQVNDVDEKALERQRKIIERAQEIHRRQQEILEELERIIRKPGSSEEAMKRMLKLLEESLRLLKELLELSEESAQLLYEAR

SEQ ID NO: 59 - protein, 35 aa, synthetic construct
EAMKRMLKLLEESLRLLKELLELSEESAQLLYEAR

SEQ ID NO: 60 - protein, 116 aa, synthetic construct
TTKRYLEEAERAHREQKEIIKKAQELHRRLEEIVRQSGSSEEAKKEAKKILEEIRELSKRSLELLREILYLSQQVNDVDEKALERQRKIIERAQEIHRRQQEILEELERIIRKPGS

SEQ ID NO: 61 - protein, 77 aa, synthetic construct
TREELLRENIELAKEHIEIMREILELLQKMEELLEKARGADEDVAKTIKELLRRLKEIIERNQRIAKEHEYIARERS

SEQ ID NO: 62 - protein, 78 aa, synthetic construct
GTERKLLERSRRLQEESKRLLDEMAEIMRRIKKLLKKARGADEKVLDELRKIIERIRELLDRSRKIHERSEEIAYKEE

SEQ ID NO: 63 - protein, 76 aa, synthetic construct
GDRQELIRRNIELLKEHIKILEEISQLIEELSELLDKSSSEEVVKRYKKILERYKQLLRKSQEIHKESSEIAKKES

SEQ ID NO: 64 - protein, 82 aa, synthetic construct
GDEQKLIERSQRMQKESLELLKEIIKILDTIEKLLDKPDSEELLDTIKKLHDTLKKIHDRNKKLLKEHEEILRQRSGSLVPR

SEQ ID NO: 65 - protein, 73 aa, synthetic construct
DKEEEYKRLLDEIKEILKESKEVLKDSKRVLEDIKRKVPDDDLVKLLEKHVRLLEEHVKLLEQLIREAEKSSK

SEQ ID NO: 66 - protein, 72 aa, synthetic construct
QGSSAEELLKKIKESEKKIRDSLRKIKEIIKKSRKEGVDDKQLDLIRKVVESHRDLLRLHRDLLRLLREETS

SEQ ID NO: 67 - protein, 77 aa, synthetic construct
DIDESIKEVEKLLEEVEQSLQKLDDSLKKLLEKVNQDPDVDDSVRKIVKRHVEILKRHEEVLKRLIEVVKEHTKTVK

SEQ ID NO: 68 - protein, 72 aa, synthetic construct
GSDREEVHKEIVKLIREIIKIHKKILKIHEKIKNGEIDPSEILKLSEEIKKLTDTIIKIIEDLEQLTRDLRR

SEQ ID NO: 69 - protein, 71 aa, synthetic construct
DRKEIVKRHQKVVELLKESSKLLRESSKLLQRLLDKTGDENLQKAVDDQDKAIKRQETAIRKSQEASKKLD

SEQ ID NO: 70 - protein, 77 aa, synthetic construct
DNSEEIKKVAKTSREVAEYSERVAKENDKVVKTLEEGKIDESELLRLLEESIKIFDTALKLHEEAYKLHQDLVRKVS

SEQ ID NO: 71 - protein, 72 aa, synthetic construct
DESEAASVAIESVQILVESVKLLEESVRILLDAVKKNGVEDLLRVAQRWEKLVDEWLKVVKRWLDNVRDIQR

SEQ ID NO: 72 - protein, 76 aa, synthetic construct
GSDKAEEVEKSVRKIEESIKKIRKSIKKAEDAVQLLKEGKIDAKDFLRIVREDLEVVKEDVEIVKEDVENVREFSS

SEQ ID NO: 73 - protein, 69 aa, synthetic construct
SDKEVSDKLLKASKKLLKVSEELLEVVRRLLKALKDDELIKKIADLLRKIIDKDKKFIRTSEEIVKESR

SEQ ID NO: 74 - protein, 78 aa, synthetic construct
GSDLKEVLKTVEEAVKEIIKSSEELLQISRKILEISRVGVDEHEYISAIREYLKALEKHIQILKKFIEILKELIRAVS

SEQ ID NO: 75 - protein, 81 aa, synthetic construct
SKEEIDKIVKKHKKKIEEHKKKVDELKKLVEEHDKRVSQDKDDKVKKLSEEVKKIIKRLEEVSKRLEEVSKKLLKVISDKR

SEQ ID NO: 76 - protein, 78 aa, synthetic construct
GSNDEELKKILETLDRILKKLDKILTRLIEVLKKSEDPNLDDKDYTELVKQFIELIKKYEEVVKEYEEVVRQLIRLFS

SEQ ID NO: 77 - protein, 81 aa, synthetic construct
SKEEIDKIVKKHKKKIEELKKLVDELKKLVEEHDKRVSQDKDDKVKKLSEEVKKIIKRVEEVAKRLEEVSKKLLKVISDKR

SEQ ID NO: 78 - protein, 78 aa, synthetic construct
GSNDEELKKILETLDRILKKLEKILTRLIEVLKKSEDPNLDDKDYTELVKQFIELIKKFEEVIKEYEEVVRQLIRLFS

SEQ ID NO: 79 - protein, 81 aa, synthetic construct
SKEEIDKIVKKHKKKIEEHKKKVDEHKKLVEEHDKRVSQDKDDKVKKLSEELKKISKRLEEVSKRLEEVSKKLLKVISDKR

SEQ ID NO: 80 - protein, 78 aa, synthetic construct
GSNDEELKKILETLDRILKKLDKILTRLDEVLKKSEDPNLDDKDYTELVKQYIELVKKYEEVVKEYEEVVRQLIRLFS

SEQ ID NO: 81 - protein, 69 aa, synthetic construct
DHSRKLKEILDRLRKHVKRLKEHLDELRDLVRQVPEDKLLEHVVKLSDKILQISERAVREFTKSVDKDS

SEQ ID NO: 82 - protein, 76 aa, synthetic construct
GSDKKDELERILDEIRRLIERLDEILSRLNKLLELLKHGVPNAKEVVKDYIRLLKEYLELVKEFLKLVKRHADLVS

SEQ ID NO: 83 - protein, 72 aa, synthetic construct
DSDEHLKKLKTFLENLRRHLDRLDKHIKQLRDILSENPEDERVKDVIDLSERSVRIVKTVIKIFEDSVRKKE

SEQ ID NO: 84 - protein, 75 aa, synthetic construct
GSDDKELDKLLDTLEKILQTATKIIDDANKLLEKLRRSERKDPKVVETYVELLKRHEKAVKELLEIAKTHAKKVE

SEQ ID NO: 85 - protein, 73 aa, synthetic construct
MDEEDHLKKLKTHLEKLERHLKLLEDHAKKLEDILKERPEDSAVKESIDELRRSIELVRESIEIFRQSVEEEE

SEQ ID NO: 86 - protein, 74 aa, synthetic construct
GDVKELTKILDTLTKILETATKVIKDATKLLEEHRKSDKPDPRLIETHKKLVEEHETLVRQHKELAEEHLKRTR

SEQ ID NO: 87 - protein, 72 aa, synthetic construct
DSDEHLKKLKTFLENLRRHLDRLDKLLKELRDILSENPEDERVKDVIDELERVIRIVKTVIKIFEDSVRKKE

SEQ ID NO: 88 - protein, 75 aa, synthetic construct
GSDDKELDKLLDTLEKILQTATKIIDDLNKVLEKLRRSERKDPKVIETVVELLKRHEKAVKELLEIAKTHAKKVE

SEQ ID NO: 89 - protein, 72 aa, synthetic construct
DSDEHLKKLKTFLENLRRLEDLLDKHIKQLRDILSENPEDERVKDVIDLSERVVRTVKTVIKIFEDSVRKKE

SEQ ID NO: 90 - protein, 75 aa, synthetic construct
GSDDKELDKLLDTLEKILQTATKVVDDANKLLEKLRRSERKDPKVVETYVELLKRLEKLIKELLEIAKTHAKKVE

SEQ ID NO: 91 - protein, 37 aa, synthetic construct
DSDEHLKKLKTFLENLRRHLDRLDKHIKQLRDILSEN

SEQ ID NO: 92 - protein, 114 aa, synthetic construct
EDERVKDVIDLSERSVRIVKTVIKIFEDSVRKLEKTKPDSKTAKELDKLLDTLEKILQTATKIIDDANKLLEKLRRSERKDPKVVETYVELLKRHEKAVKELLEIAKTHAKKVE

SEQ ID NO: 93 - protein, 112 aa, synthetic construct
DSDEHLYKLKTFLENLRRHLDRLDKHIKQLRDILSENPEDERVKDAIDLSERSVRIVKTVIKIFEDSVRKKEKRPIDKRDDKELDKLLDTLEKILQTATKIIDDANKLLEYL

SEQ ID NO: 94 - protein, 35 aa, synthetic construct
GDPKVVETYVELLKRHEKAVKELLEIAKTHAKKVE

SEQ ID NO: 95 - protein, 72 aa, synthetic construct
DSDEHLDRLDKHLKKLKTFLENLRRHIKQLRDILSENPEDERVKDVIDLSKTVIKIFEDSVRKKERSVRIVE

SEQ ID NO: 96 - protein, 75 aa, synthetic construct
GSDDKEATKIIDDLDKLLDTLEKILQTANKLLEKLRRSERKDPKVVETYVKAVKELLEIAKTHAELLKRHEKKVE

SEQ ID NO: 97 - protein, 72 aa, synthetic construct
DSDEHIKQLRDHLDRLDKHLKKLKTFLENLRRILSENPEDERVKTVIKIFEDSVRKKERSVRIVKDVIDLSE

SEQ ID NO: 98 - protein, 75 aa, synthetic construct
GSDDKEANKLLEKATKIIDDLDKLLDTLEKILQTLRRSERKDPKAVKELLEIAKTHAELLKRHEKVVETYVKKVE

SEQ ID NO: 99 - protein, 104 aa, synthetic construct
DHSRKLEEILDRLRKHVKRLLEHLRELLSLVKENPEDKDLVEVLELSLAILRRSLEAVEAFLKSVTKKDPDDEDLRRKADEIRKEVEEIKKSLAEVEKEIYKLK

SEQ ID NO: 100 - protein, 116 aa, synthetic construct
GSSADDVLEDILKIIRELIEILDQILSLLNQLLKLLRHGVPNAKKVVEKYKEILELYLQLVSLFLKIVKTHADAVSGKIDKKAEEEIKKEEEKIKEKLRQAKDILKKLQEEIDKTR

SEQ ID NO: 101 - protein, 107 aa, synthetic construct
DRDAHLYKLLTFLEQLVRHLDRLVKHITQLRDIVKKDPEDERAVDVIRQSVRSLEIVITVLKIFVDSVSDAARSKEAEKIVRKIRKEIDEIRQKLREIDKEVKKTTS

SEQ ID NO: 102 - protein, 114 aa, synthetic construct
GSNDKVLDKILDILDRILRLATRVIDLANKLLQVKKKSTHKDPRIVETYKELLKIHETAVRLLLELADLHRRLKSKDEEANKRVETELDRIRKKVKDIEDKVRKLEDKVRKTAS

SEQ ID NO: 103 - protein, 68 aa, synthetic construct
NDLSKEVLKKLEKSVEELLRRVQKSVKEAQKRGLLSDELVDRHLKILNQLVKRHLELLQEVIKRSDKK

SEQ ID NO: 104 - protein, 78 aa, synthetic construct
GSDEAVKRVVEKSLKILDEVIKKSLDILRELIELQIRHAKDDESVIRASKSALKDAIEALKKSLDEIKKALKRSADEG

SEQ ID NO: 105 - protein, 76 aa, synthetic construct
SSEEVVKVHEKVVKLHKEILELLKKIIKIHETAARDPDDKDSIKKLSDEIKKIVKRIEDISDQAKRESSDAQRKQS

SEQ ID NO: 106 - protein, 74 aa, synthetic construct
DKEEESKELLKKLKEILKRSEELLEESKELLKLAKNGEIDESELADADRKLNKKHEKLVQDIQDLLREHERQDR

SEQ ID NO: 107 - protein, 73 aa, synthetic construct
DEKKKIDKIVKETEDLLQKSEKLLQQSKEAVKRIRSQVKENEIVDRLLRISEELLKISRRLVEISRRIASTLS

SEQ ID NO: 108 - protein, 78 aa, synthetic construct
GSSKEEVIRLLKENVRLIKENLELLTRNLKLITDLVRGSNGSEEKIKTLKELLKEYRELLKRYRKLVEDYKRLVDKHD

SEQ ID NO: 109 - protein, 81 aa, synthetic construct
EIQELIKSSRRIIEESKELIKESEEVLRRIKEILDRIRNGVDNQEDLLREILKLLTKNLKIIQRNLKLLQDNAEILKRLVS

SEQ ID NO: 110 - protein, 75 aa, synthetic construct
GSYIEDVIKKILDVSRELIKLSRTIIKISEEINKQLQQGRDTKDLVKKYDEIIKKYTRIVQHYTELIKELQKLLS

SEQ ID NO: 111 - protein, 75 aa, synthetic construct
SPTEEAIQLSQRVIELSKRVIELSKEILKLLKRVLDLLPDLDKNEEKRLDDYDKELKEYDKELKKYEKRLKDLAS

SEQ ID NO: 112 - protein, 75 aa, synthetic construct
GSEEEEILKIQKELLRIQSEILDKQKKILDTLRSNGAVTEEVRSILEKVERLSEEAKELSKEAKELTKEVSKLIS

SEQ ID NO: 113 - protein, 77 aa, synthetic construct
SPLKELNNQLLRLLRELVKVSKKIVDLSKTIIEVLKHTDLDPRLLDSLEKSQQELDKSQKELDKVVKELTKVNKKLQ

SEQ ID NO: 114 - protein, 78 aa, synthetic construct
GSPLEDLVRKYDELVKTYEKLVEEFKKAVDKYDKAVKKAPVSKEATDSLDLIRKVLELLDRNLKLIKENAKLIKELLK

SEQ ID NO: 115 - protein, 69 aa, synthetic construct
SPTRENEKVIKENEKVISDNERVLEEVVKVVETATDRKEIQDAVDEVRKSVDKLRDSVRKLEESVRTLD

SEQ ID NO: 116 - protein, 72 aa, synthetic construct
GSPIKDISKRLLEISKRLVEISDRIVELLQRIADSKDPNKDLQKEVKDVLEEYKRLVREYREVVKEYEKVVS

SEQ ID NO: 117 - protein, 75 aa, synthetic construct
DEDEHVKQLIKNADLLRKHAELLKELVKLFQEIASQIPDDRVAKKVTDVVDRIDKILKQTEKLVRRTKQILDYSR

SEQ ID NO: 118 - protein, 71 aa, synthetic construct
GSNLEELVKLLKEVLEMHERLLRIHEDLVEAHKSNASDKESERKLKKSDKDIKESLKKIKSIIDQVRYIQS

SEQ ID NO: 119 - protein, 70 aa, synthetic construct
PVEDIIEESLRLLEESLKLLNRILKLLEDSLRKLPRSEEWRQRLDEFRKKLEDWKEELERWIEDVRYKKT

SEQ ID NO: 120 - protein, 78 aa, synthetic construct
GSDEDYESREIIDEIRKLLDRSKKIVHRSQRLVERVKSTPLSEDQEDLIRRHEETINRHRELVKELEKVLEDHERHIR

SEQ ID NO: 121 - protein, 78 aa, synthetic construct
PEEDSRRVLERFVRVSREVLKVLEEFLRVSEELLREADRDRDRRLEEYERQVDELREEIRRYKEEVDKFDKEVKYYKK

SEQ ID NO: 122 - protein, 79 aa, synthetic construct
GSPEKDENRKLLDKVRKLVEKSRRLVEELRKLVDQSTKNGLIDEKALRKQQEVLRKVEEVLEKQERVLRELEEISYRVI

SEQ ID NO: 123 - protein, 39 aa, synthetic construct
GSPERDENRKLLDKVRKLVEKSRRLVEELRKLVDQSTKN

SEQ ID NO: 124 - protein, 124 aa, synthetic construct
GSDEKALRKQQEVLRKVEEVLEKQERVLRELEEISYRVITRGEDHKAEEDSRRVLERFVRVSREVLKVLEEFLRVSEELLREADRDRDRRLEEYERQVDELREEIRRYKEEVDKFDKEVKYYKK

SEQ ID NO: 125 - protein, 39 aa, synthetic construct
GSDRRLEEYERQVDELREEIRRYKEEVDKFDKEVKYYKK

SEQ ID NO: 126 - protein, 125 aa, synthetic construct
GSPERDENRKLLDKVRKLVEKSRRLVEELRKLVDQSTKNGLIDEKALRKQQEVLRKVEEVLEKQERVLRELEEISYRVITRGEDHKAEEDSRRVLERFVRVSREVLKVLEEFLRVSEELLREADR

SEQ ID NO: 127 - protein, 75 aa, synthetic construct
DLSEESKKFVEKVKKLEKESRELEKQVKKIEEDSRSVENDVQKEFLELLKRLLDIQKKVVEVLREVVKVQQYVDS

SEQ ID NO: 128 - protein, 80 aa, synthetic construct
GSDSEYESRQVLRELDTVLKDSHTVLEALRQVIRDSQDVVSKSDEESRRVIDDLEKVIQDSKKVLDDIKRLIDKSKSIKS

SEQ ID NO: 129 - protein, 75 aa, synthetic construct
NEDELLKLLTENLKLLDENLKLLRENLSLLRQANNITDKNRIREIVKQSKEIVKQSREILKQSKEIVERIKYIVS

SEQ ID NO: 130 - protein, 78 aa, synthetic construct
GSSLYELTQRYEKLVQQYEELVKDYRRLVKKLEKLKRDNKPDKRLLKEIVDVIKKSVEIIDRSLKLLEESIKILEETD

SEQ ID NO: 131 - protein, 73 aa, synthetic construct
SQERSLEILKRILDVLKESLEILKESLSILRQLASRIKNPNRKIEEILKESDKIIKESDKVLKEIEEVIRYSS

SEQ ID NO: 132 - protein, 78 aa, synthetic construct
GSDIEYESKEILELIKELLKLSRELLKESRRALELVRKSRDDSIVEEVIQVHKKVLDIHKEVLKIVRKVVEVHRRVKS

SEQ ID NO: 133 - protein, 76 aa, synthetic construct
SKKDESTKLERLAEKIDEITKRIEELVKDVKRKSSEGVDKDQQQKIDEVFQKLLDLQREILEILDRILKVQQYILD

SEQ ID NO: 134 - protein, 78 aa, synthetic construct
GSDLEYLNRRLLQLIKTLIDLNRHLLKLIDKLKKLNSREGDEEKIKEESKQIQEQFKEIVERSKEIIKQIKEIIKRSQ

SEQ ID NO: 135 - protein, 79 aa, synthetic construct
DFERSSRRLEKVVEDLRRSSDRLREVIDELRKSADEKDEDEDLRRARKEHRDLIEELKRALEKQEEIIKHLQELVYRQL

SEQ ID NO: 136 - protein, 78 aa, synthetic construct
GSEESEEVRKVVERIKKISRELEEVVKELDRVSKEFDRHGETDEIVREHERIVEKLEEIVKKHTKIVEELAEIVYKQQ

SEQ ID NO: 137 - protein, 71 aa, synthetic construct
SDDDSVRVLDEIVKILDESVKLLKESLKLLDDFLRTKPDDHLKEVVKESKKVVEQSKKVLDRIKKIIYESK

SEQ ID NO: 138 - protein, 72 aa, synthetic construct
GSDLLYLSKELLKLVRELLKLSRELVELSRRLVNSTHKSPELVKKYDKLVKKYQDLLKKLADVADEYLRQRS

SEQ ID NO: 139 - protein, 76 aa, synthetic construct
DEKDYHRRLIEHLEDLVRRHEELIKRQKKVVEELERRGLDERLRRVVDRFRRSSERWEEVIERFRQVVDKLRKSVE

SEQ ID NO: 140 - protein, 78 aa, synthetic construct
GSDAYDLDRIVKEHRRLVEEQRELVEELEKLVRRQEDHRVDKKESHEILERLERIIRRSTRILTELEKLTDEFERRTR

SEQ ID NO: 141 - protein, 77 aa, synthetic construct
DERYRAREHIRRVEEHTKRLRHILKRLREHEEKLRRELKPGDEITESVDRFKKIVDQFEESIKKFETVSEELRKSDS

SEQ ID NO: 142 - protein, 71 aa, synthetic construct
GSDRQRILDRLDKILEKLDDILKKLKDILETLSKDDVSDRRHKDLVEKFRELVDTHHKLVERYRELVYQNR

SEQ ID NO: 143 - protein, 38 aa, synthetic construct
GSDEITESVDRFKKIVDQFEESIKKFETVSEELRKSIS

SEQ ID NO: 144 - protein, 107 aa, synthetic construct
GSDPQRAADRLDKILEKLDDILKKLKDILETLSKDDVKDRRAKDLVEKFRELVDTHHKLVERYRELVYTATAGSDLARELIRRVEEHTKRLRHILKRLREHEEKLRR

SEQ ID NO: 145 - protein, 73 aa, synthetic construct
NADDQLATSIKKLEDSIDQLIKIVRKFEESVKKLQKHGVDQHHVEILRKIVEIFRQHIEKLKKHLEKLRYTSS

SEQ ID NO: 146 - protein, 70 aa, synthetic construct
GSDKEYLVTEHEKLVREHEKIVSEIEKLVKKHEAGVDESELEEILKKVEKLLRKLDEILEQLTQLLRKTE

SEQ ID NO: 147 - protein, 36 aa, synthetic construct
GSDQHVVEILRKIVEIFRQHIEKLKKHLEKLRYTSS

SEQ ID NO: 148 - protein, 114 aa, synthetic construct
GSDAEYLVTEHEKLVREHEKIVSEIEKLVKKHEKGVDESELEEILKKVEKLLRKLDEILEQLTQLLRKAEKHIDKHSKAADQLATSIKKLEDSIDQLIKIVRKFEESVKKLQKH

SEQ ID NO: 149 - protein, 74 aa, synthetic construct
DEDDDIRRVLDESRRVLEHSRRVLKRSEEVLEKASRKKEKDTEEIEKHLKRLREHAKKLEKHRRELDDFLYKEI

SEQ ID NO: 150 - protein, 74 aa, synthetic construct
GSRDKYLLERLNDILKKLDEIVDKLSDILKRLKDVRHDDRLQELVERYKEIVKEYKRIVEEYEKLVREFEEQQR

SEQ ID NO: 151 - protein, 73 aa, synthetic construct
DRDYEDKEFKKIIKELEDVQEELKKLQEKIKRFSSELEEPNELLKEQLKVNEEQLEVNKKILKILRDQLKQNE

SEQ ID NO: 152 - protein, 76 aa, synthetic construct
GSDAEYKVRESVKRSKESVKHSEDVVDKLNKSVKLSESGHSDAEKASRELVKLVREVVELSREVIKLSEKVLRVIS

SEQ ID NO: 153 - protein, 79 aa, synthetic construct
DLQYKQEKLIRHFDRVVREWDKLVRKFSKVLEKQKHESKDKELEEASRRVDELIKRLREQLKRSKEILRRLKELSRKSS

SEQ ID NO: 154 - protein, 79 aa, synthetic construct
GSDWEELLRRLEKVLQEYEEIVKELIDLIERLIKVSEDKSKDASEYKKLVTELEKLISKLEEISKKLEELVKEYEYKTE

SEQ ID NO: 155 - protein, 76 aa, synthetic construct
DAKDELEKSLQEIEESLKELKKLLEELDKSLRELTSQGRNKKLEEHIKKVQKFIELVKKYIKAVQDYLKEVRYDNS

SEQ ID NO: 156 - protein, 75 aa, synthetic construct
GSDKERAARATEEMVKLTKKLLKAVEDLVRDVRRLLKEGLISEKHARIAETILEVFKKHAKIIKKHVDIVKYDES

SEQ ID NO: 157 - protein, 78 aa, synthetic construct
GSPLKERLLEIQRDLDRVLEEVVERLLRIQERLDSVVERKPPDVHEEYKYIVDEIREIVERVVREYEEIVKRIDEEVR

SEQ ID NO: 158 - protein, 83 aa, synthetic construct
GSEEDERIRYDLDRIRKDVRRKLEEIRQRVRELEKKLRDAGHRRDEKELLRELIETSKDILRLVEELLKKIIDKSEDLLRKTE

SEQ ID NO: 159 - protein, 73 aa, synthetic construct
GSDEEDYINENVEKDVRDIEDDVRRINERIRELLEKIRTEEVLQRVLEEHHELVERVLRKLVEILRKHEEENR

SEQ ID NO: 160 - protein, 77 aa, synthetic construct
GSDEEEYYKEKLHKLLREIEELLKHYRELVRRLEELVKRGELDKDTAAHILERLSELLERIIRRVAHTLRRLSEERR

SEQ ID NO: 161 - protein, 84 aa, synthetic construct
GSDEDEISYDSKRRVEEIVRQAREKSEKSRKDIEDVAEVLRKGDVSEKEVVDELVKVLEEQVKVLREAVERLREVLKKQVDDVR

SEQ ID NO: 162 - protein, 82 aa, synthetic construct
GSDIVELVDHLLKRSLKLLEELAELVRRLLEKSTELLKRRTEEHKEEVVEESEYMVRELEERLRRVVDESEKLVRDADKHIR

SEQ ID NO: 163 - protein, 83 aa, synthetic construct
GSKEKDIVKTLVDLLRENLETLERLIEEVVRLLKENVDVRDEGRDDKDSERILRDIKRRIDEAAKESREIIERIEKEVEYRSR

SEQ ID NO: 164 - protein, 80 aa, synthetic construct
GSPEVDVLRRIVREILKASEELLRLLRKLIDEALKLSERKRDSQEYREVVDRVKKELERLLDEYRKLVEELKEKLRYDTR

SEQ ID NO: 165 - protein, 84 aa, synthetic construct
GSDKRYESEKLKRRLDEAVEKVREVVERVERESDRVLEEVRRRRESKEVVDKVIEDNDKALEDVLRVVDEVAKVVRDVVRENTR

SEQ ID NO: 166 - protein, 77 aa, synthetic construct
GSPREYHSKDILRKVDEILERIRRHADRVKKKSERLKRENVDVNEHSKDVKRVIRELLELVKELLRLAKKHSDDQQE

SEQ ID NO: 167 - protein, 79 aa, synthetic construct
GSDEDEILYHSERLLQKLKKELDDLKEKSRELLEELKKEDPDDRLIERIIRLHDEVLKDLDEVLKNILEVHREVLERLR

SEQ ID NO: 168 - protein, 75 aa, synthetic construct
DKLDRLLKIHEEALRRAEELIKRLLDIHRRALDLARRGELDDYLLKESERELREIIRRAREELKESRDRLEEISR

SEQ ID NO: 169 - protein, 80 aa, synthetic construct
GSPKEELIRRVLEEVKRLNEKLLEIIRRAAELVKRANDELPETEKLREIDRELEKKLKEIEDELRRIDKELDDALYEIED

SEQ ID NO: 170 - protein, 80 aa, synthetic construct
GSPKLDKLRELLERNLEKLREILEEVLKILRTNLERVREDIRDEDVLQEYERLIRKAEEDLRRVLKEYDDLLKKLVYELR

SEQ ID NO: 171 - protein, 75 aa, synthetic construct
GSKEDESVKRAEEIVRTLLKLLEDSLREAERSLRDIKNGEDEHNLRRISEKLEELSKRITETIERLLRELQYTSR

SEQ ID NO: 172 - protein, 84 aa, synthetic construct
GSPNQELLDRVRKILEDLLRLNEELVRLNKELLKRALEMRRKNRDSEEVLERLAEEYRKRLEEYRRELEKLLEELEETIYRYKR

SEQ ID NO: 173 - protein, 84 aa, synthetic construct
GSDESEEAQHEVEKVLDDIRRLSEHLQKRLEEVLEEVYELRREGSDRTEVVELLKEVIREIVRVNREALERLLRVVEEAVKRNE

SEQ ID NO: 174 - protein, 83 aa, synthetic construct
GSDEEELVETVKRIQKEILDRLTELAKLLVEIQREIKKLKDEGEDDKELKRLSDELEEKVRQVVEEIKRLSDELEETVEYVSR

SEQ ID NO: 175 - protein, 80 aa, synthetic construct
GSDEEEEVVRRAEELVKEHEELIERVIRTHEELVYKLEDQGADKKLVDVLKRVVEESERVAREIVKVSRELIRLLEEASR

SEQ ID NO: 176 - protein, 80 aa, synthetic construct
GSSKEEILKELEDLQRRLIEELKKLQERVVELLEELIKRLRDRGRDDKHLKRLVKEVRRLSEEVLRSIKEVSDRVRYQLR

SEQ ID NO: 177 - protein, 81 aa, synthetic construct
GSDKEEESEYLLRDLVRLLEKVKEKIEEVNREVEKLLKKVKDGRLDRREVLREILRLNRELAEIIKEVVDRIRHVVERSER

SEQ ID NO: 178 - protein, 76 aa, synthetic construct
GSDLHEVVYETKELLKRIEEVVEELRKKSEDIIRKAERGEISEDELKRLQEEIAREAKKLLDEIKRVLERHLEQTL

SEQ ID NO: 179 - protein, 81 aa, synthetic construct
GSPVEEIIKEVVKRVIEVQEKVLRIISHAVKRVVEVQKKYDPGSEESNRVVEEVKKTIEDAIRESDEVVDEVVKRIQYTVR

SEQ ID NO: 180 - protein, 80 aa, synthetic construct
GSPEQEIADRILTEIRESQKELERLARKILKLLDESQEKAKRGRLSEEESDELLERIKKELDELLERSKELLKKIEYELR

SEQ ID NO: 181 - protein, 80 aa, synthetic construct
GSDEDKEANRVLDEVLKTVRDLLETANEVLKEVLYRLKRTDDQEKVVRTLTEVLKEHLKLVEEIVRILDKVLKEHLETEK

SEQ ID NO: 182 - protein, 76 aa, synthetic construct
GSPEDDVLRRLEEVSEKILRVAEDVARQLREVSEKITQGKVDRKEWEEDIKRLKRELEELLREWKEEIERLTYELR

SEQ ID NO: 183 - protein, 78 aa, synthetic construct
GSRREEVVKRIRELLKRNKELIDRIRELLEENEYLDKDARDKDVLRRSVELLEELVRILEESVELAKEIIKLLREVVE

SEQ ID NO: 184 - protein, 75 aa, synthetic construct
GSDEKEDNRRLQHKIERILEKNEDLQRKLEEILELLERGEADEEKIDRLRKAVEDYRRVVEEIKEDVKRHKYTVR

SEQ ID NO: 185 - protein, 77 aa, synthetic construct
GSDEKEEAKKASEESVRTVERILEELLKASEESVELLRRGEDAKDVVERSKEALKRVKELLDEVVKRSDEILKYIHN

SEQ ID NO: 186 - protein, 84 aa, synthetic construct
GSDEKKLINEVVETQKRLIKEAAKRLSEVVRHQTELIRELREKNVDDKDVEKLLKESLDLAEEIVRRIKELLDESKKLVEYVSN

SEQ ID NO: 187 - protein, 76 aa, synthetic construct
GSPDMDEVKRVLDELIEIQEEILREIKRVLEKLIKIQEDNGSEYESREVVREIVEIARKLVERSRRVVKKITETLQ

SEQ ID NO: 188 - protein, 80 aa, synthetic construct
GSDERYATREIVERIERIAREILKRTEEIVREVREVLSRDVDQEEVVRRLADLLRESVELVQHLVRRVEELLQESVERKK

SEQ ID NO: 189 - protein, 79 aa, synthetic construct
GSPEREALREVLEDLKRVTDRLRELVERVLEELKKVTDHVDSERILRESRRVLKELKDIIEEILRESEKVLEKLKYTED

SEQ ID NO: 190 - protein, 80 aa, synthetic construct
GSPAREILEEVVKKHLEVVEDAARILEEIIREHEKAVREDRDKKELEEISRDLLRKAREALKKVKDISDDLSREIEYVAS

SEQ ID NO: 191 - protein, 75 aa, synthetic construct
GSPVEEAIKKVIDDLRDVQRKIRELVEELIRLLEEVQRDNDKRESEYVVERVEEILRRITETSREVVRKAVEDLS

SEQ ID NO: 192 - protein, 81 aa, synthetic construct
GSDSDEKAEYLLKEMERVVRESDEVVKKILRDLEEVLERLRRGEISEDDVTEILKELAERHIRAIEELVRRLRELLERHKR

SEQ ID NO: 193 - protein, 79 aa, synthetic construct
GSPVEEVLKELSEVNERVRDIAREIIERLSEVNEEVKETDDEDELKKISKKVVDEVEDLLRKILEVSEEVVRRVEYHDR

SEQ ID NO: 194 - protein, 76 aa, synthetic construct
GSPKEDILREVLRRHKEIVREIVRLVREAVETHLELVKRNSDDRDAQDVIRKLEEDLERLVRHAQEVIEEIFYRLH

SEQ ID NO: 195 - protein, 75 aa, synthetic construct
GSPRSYLLKELADLSQHLVRLLERLVRESERVVEVLERGEVDEEELKRLEDLHRELEKAVREVRETHREIRERSR

SEQ ID NO: 196 - protein, 76 aa, synthetic construct
GSDREYIIKDILDSQEHLLRLIEELLETQKELLEILKRRPDSVERVRELVRRSKEIADEIRRQSDRNVRLLEEVSK

SEQ ID NO: 197 - protein, 75 aa, synthetic construct
GSDEKDEIRHVIESVERLIEDIKRLLKTLRELAHDDSDKKTVKEVLDRVKEMIERHRRELEEHRKELERAEYEVR

SEQ ID NO: 198 - protein, 76 aa, synthetic construct
GSESEDRIKELLKRHIELVERHEELLHEIKKLIDLEEKDDKDREEAVKRIDDAIKESEEMLEESKEILEEIEYLNR

SEQ ID NO: 199 - protein, 77 aa, synthetic construct
GSSLEDSVRLNDEVVKVVERVVRLNQEVVRLIKHATDVEDEETVKYVLERVREVLDESREVLKRVHELLEESERRLE

SEQ ID NO: 200 - protein, 82 aa, synthetic construct
GSHEKDIVYKVEDLVRKSDRIAERAREIVKRSRDIMREIRKDKDNKKLSDDLLKVTRDLQRVVDELEELSRELLRVAEESRK

SEQ ID NO: 201 - protein, 76 aa, synthetic construct
GSPELDEVKKLIDELKKSVERLEESIREVKESIKKLRKGDIDAEENIKLLKENIKIVRENIKIIKEIIDVVQYVLR

SEQ ID NO: 202 - protein, 82 aa, synthetic construct
GSDEEEIEELLRELEKLLKKSEEALEESKKLIDESEELLRRDRLDKEKHVRASEEHVKLSEEHLRISREIVKILEKAVYSTR

SEQ ID NO: 203 - protein, 79 aa, synthetic construct
GSDESDRIRKIVEESDEIVKESRKLAERARELIKESEDKRVSEERNERLLEELLRILDENAELLKRNLELLKEVLYRTR

SEQ ID NO: 204 - protein, 79 aa, synthetic construct
GSDEDDELERLLREYHRVLREYEKLLEELRRLYEEYKRGEVSEEESDRILREIKEILDKSERLWDLSEEVWRTLLYQAE

SEQ ID NO: 205 - protein, 78 aa, synthetic construct
GSDKKDASRRAIRVLHEFVRVSEEVLEVLRKSVESLKRLDVDEKIKRTHDRIEEELRRWKRELEELIERLREWEYHQD

SEQ ID NO: 206 - protein, 75 aa, synthetic construct
GSDDEEEDKRLLEEVKRSLDTDERILEKLRHSLERQLEDVDKDEDSRRVLRELDEITKRSREVVKRLRKLAYESK

SEQ ID NO: 207 - protein, 79 aa, synthetic construct
GSDKEYKLDRILRRLDELIKQLSRILEEIERLVDELEREPLDDKEVQDVIERIVELIDEHLELLKEYIKLLEEYIKTTK

SEQ ID NO: 208 - protein, 79 aa, synthetic construct
GSPSKEYQEKSAERQKELLHEYEKLVRHLRELVEKLQRRELDKEEVLRRLVEILERLKDLHKKIEDAHRKNEEAHKENK

SEQ ID NO: 209 - protein, 78 aa, synthetic construct
GSRDRKISEELIKALEDHIRMLEELIRAIEEHIKLAERGVDEKELRESLEELKKIVDELEKSLEELRKLAERYKYETR

SEQ ID NO: 210 - protein, 77 aa, synthetic construct
GSPKEESVEELKRVIDKHEEILRELKRVLEEHERVSHDEDENELRRSLERLKHILDRLHESLKELHELLKKNEYTER

SEQ ID NO: 211 - protein, 77 aa, synthetic construct
GSDHEYWVKIVERILRVMEKHAEIVKKHLEIVERVVREGPSEDLRRKLKESLREIEESLRELKELLDELDELSEKTR

SEQ ID NO: 212 - protein, 72 aa, synthetic construct
GSDEEYVTRSQRRLKRLLEEYIKVVEEHARLVERNERDDKELKRSIDELDKLTKELLELVKRYKELVDKTET

SEQ ID NO: 213 - protein, 76 aa, synthetic construct
GSDKEEIVKLQDEVIKTLERHLDILRKHIDLLEKLKDHLSEELKERVDRSIKKLEESIKRLERIIEELQELAEYSL

SEQ ID NO: 214 - protein, 75 aa, synthetic construct
GSREEELKESAEELERSVRELKKEADKYKEEVDRLHYRGKVDKDWVRVVEKLIKLVEEHLELIREHLELLKEERR

SEQ ID NO: 215 - protein, 75 aa, synthetic construct
GSDMEYELKKSAEELRKSLEELKRILDELHKSLRELRRHGDDEEYVQTVEELRKELEEHAKKLEEHLKELERVAT

SEQ ID NO: 216 - protein, 70 aa, synthetic construct
PEYELKKSVDDLKRDVDRLVEEVEEVFELSKERLREDRKHLELVEEMVRLIEKHLELIKEHLKLADDHVR

SEQ ID NO: 217 - protein, 78 aa, synthetic construct
GSREKDESKELNDEYKKLLEEYERLLRRSEELVKRAKGPRDEKELKRILEENEDILRRTKEILERTKEISEEQKYRRR

SEQ ID NO: 218 - protein, 78 aa, synthetic construct
GSDKDERQERLNEESDKSNEESERSNRESEELNRRARGPNDEKELQEILDRHLELLERNQRLLDENKEILRESQYLND

SEQ ID NO: 219 - protein, 76 aa, synthetic construct
GSENKYILKEILKLLRENLKLLHDILRLLDENLEELEKHGAKDLDDYRRKIEEIRKKVEDYREKIEEIEKKVERDR

SEQ ID NO: 220 - protein, 75 aa, synthetic construct
GSESEYTQEEILELLKESIKLLREILRLLEESEELWRRENTKSERSEEIKERAKEAIKRSEEILERVKRLSDHSR

SEQ ID NO: 221 - protein, 74 aa, synthetic construct
GSDEEEANYVSDKAVKIAEDVQELLKELLELSEVVRRGEVDEDEYDRVLRKLQEVMKEYEEVLKEYEEVSRKHE

SEQ ID NO: 222 - protein, 80 aa, synthetic construct
GSPEKYLIKTQEELLRRHAEILEDLIRKVERQVDLRRKVDERDEDLKRELERSLRELERLVRESSRLVEEIRELSKEIKR

SEQ ID NO: 223 - protein, 78 aa, synthetic construct
GSDEEYELERISRESKELLERYKRLLREYQELLKELRHVKDLDRAVKIIHELMRVSKELVEISHRLLELHERLVRRRK

SEQ ID NO: 224 - protein, 79 aa, synthetic construct
GSEKEYIEKLSRKIEEDIRRSEERAKDSERLVRRLEELAKRKRLDLDDVLRVAEENLEILEDNLRILEEILKEQDKSNR

SEQ ID NO: 225 - protein, 75 aa, synthetic construct
GSPHEEVVELHERVMEISERAVELIQRIIDIIRRIREDDKDIEKLVKTIRDLVREYEELHRELEEIDEEIYKKSE

SEQ ID NO: 226 - protein, 82 aa, synthetic construct
GSDHEDVVRLHEDLVRKQEDARRVLEEIVRLAEEIVEVIKKDEKDKDRVTRLVEEIEKLVEEYKKKVDEMRKISDEIKYRSR

SEQ ID NO: 227 - protein, 79 aa, synthetic construct
GSRAREVVKRAKRIIEEWQKILEEWRRILEEWRRLLEDERVDDRDNERIIRENERVIRENEKIIRDVIRLLEELLYERR

SEQ ID NO: 228 - protein, 79 aa, synthetic construct
GSREDEELEEEIDRIRQMVEEYEELVKEYEELTEKYKQGKVDKEESKKIIEKSERLLDLSQDAVRKVKEIIRRILYTNR

SEQ ID NO: 229 - protein, 77 aa, synthetic construct
GSPKEEIVKLHDESAELHRRSVEVADEILKMHERSKDVDDERESRELSKEIERLIREVEEVSKRIKRLSEEVEYLVR

SEQ ID NO: 230 - protein, 77 aa, synthetic construct
GSPLEEILKIQRRINKIQDDINKILHEILRMQEKLNRSSDKDEVEESLRRIRELIKRIKDLSKEIEDLSREVKYRTT

SEQ ID NO: 231 - protein, 81 aa, synthetic construct
GSPEDEHVYVVREIYEVLREHAEVLEENREVIERLLEAKKRGDKSEELVKELKKSIDKLKEISRKLEEIVKELEKVSEKLK

SEQ ID NO: 232 - protein, 79 aa, synthetic construct
GSDEDETSYRILELLREIVRASRELIRLSEELLEVARRDDKDETVLETLIREYKELLDRYRRLIEELTRLVEEYEERSR

SEQ ID NO: 233 - protein, 74 aa, synthetic construct
GSTQEEINRIQHEVLRIQEEIDEILRDIVEKLKAISRGELDHEVVKDVEDKVREALEKSEELLDKSRKVEYKSE

SEQ ID NO: 234 - protein, 82 aa, synthetic construct
GSDEEELNRELLEKSKRLVDINRDIIRTAQELIEMLKDSKDGRVDEDTKRELRDKLRKLEEKLERVREELRKYEELLRYVQR

SEQ ID NO: 235 - protein, 77 aa, synthetic construct
GSDEKDRVYEILKEVQRLVKEYRDISKEIEDLVKHYEHITDDEAQEVSKELIDKSLRASEIVRELIRLIKELLDELE

SEQ ID NO: 236 - protein, 79 aa, synthetic construct
GSDEEDVLYHLRELLEELKRVSDDYERLVREIKETSERKDRDTKENKDMLDELVKAHREQEKLLERLVRLLEELFERKR

SEQ ID NO: 237 - protein, 54 aa, synthetic construct
PREQAIRISEEIIRISKKIIEILERTRSSTAREAMKWAKDSIRLAEESKYLLDK

SEQ ID NO: 238 - protein, 54 aa, synthetic construct
IEDDVKKIQDSTKKAQKETIEALERSTSSTARKQMEEQKEQIRLQKEAMYLLKK

SEQ ID NO: 239 - protein, 76 aa, synthetic construct
SREEIAKLQEEVIKLQRRVIELQKEVIELQRRAKELTSSYTKEILEIQRRIEEIQREIEEIQKRIEEIQEEIQRRT

SEQ ID NO: 240 - protein, 76 aa, synthetic construct
SDEEIKRLSEEVIQLSRRVIKMSREAIKLSREVQKLTPSYQKRIKEIADRSIELARESIEIAKRSEKIAEESQRRT

SEQ ID NO: 241 - protein, 56 aa, synthetic construct
PAKDEALKMANESLELAKKSARLIQESSSKEILERIEKIQRRIAELQDRIAYLIKK

SEQ ID NO: 242 - protein, 56 aa, synthetic construct
PAKDEALRMIDESRELIKKSNELIQRSSSKEILERILEIQRKIAELQKRIQYLLKS

SEQ ID NO: 243 - protein, 75 aa, synthetic construct
TDEARYRSERIVKEAKRLLDEARRRSEKIVREAKQRSNSEDAKRIMEENLRESEEAARRLREIIRRNLEESRETG

SEQ ID NO: 244 - protein, 75 aa, synthetic construct
TREALEYQRKMAEEIEDLLREALRRQEEMVREAKQRSLSEEFKRIMERILEEQERVMRLAKEALERILEEQKRTG

SEQ ID NO: 245 - protein, 76 aa, synthetic construct
SERTKREAKRSQEEILREAKEAMRRAKESQDHRQNRDGSNSEDLERLSQEQKRELEEVERRLKELAREQKYKLEDS

SEQ ID NO: 246 - protein, 76 aa, synthetic construct
SEDLKRILKEITERELKLMQDLMEILKKITEDENNLDSNNSEDLKRSIEKARRILDEALRKLEESARRAKYIQEDN

SEQ ID NO: 247 - protein, 76 aa, synthetic construct
TEDEIRESLKWLDEVLQELREIARESNEVLERNRQKSRSDKLREDIERYKKRMEEARKKLDDQLNKYKKRMDENRS

SEQ ID NO: 248 - protein, 76 aa, synthetic construct
TEEELKESKKFAEDLARSARRALKESKRVLEEISQASRSKKLEEIVRRYKEQVKRWQDEWDERAREYRKRMKENRS

SEQ ID NO: 249 - protein, 76 aa, synthetic construct
TKTEEIERLAREIKKLSEKVERLAQEIEELSRRVKEENSTDRELKEANREIERAIREIEKANKRMEEALRRMKYNG

SEQ ID NO: 250 - protein, 76 aa, synthetic construct
TKTEEHERLAREISKLADEHRKLAKIIEELARRIKEENLTDDELREAIRKIEDALRKNKEALKIMKEAAERNRYNT

SEQ ID NO: 251 - protein, 76 aa, synthetic construct
TKKEESRELARESEELARESEKLARKSLELARRAESSGSEEEKRRIIDENRKIIERNREIIERNKEIIEYNKELIS

SEQ ID NO: 252 - protein, 76 aa, synthetic construct
TKDEESLELNRESEELNRKSEELNRKSKELNDRAESSNSEEEEKEILREHKEILREHLEILRRHKEILRRHKYLTS

SEQ ID NO: 253 - protein, 75 aa, synthetic construct
TREELLRENIELAKEHIEIMREILELLQKMEELLERQSSEDILEELRKIIERIRELLDRSRKIHERSEEIAYKEE

SEQ ID NO: 254 - protein, 79 aa, synthetic construct
SEDIAREIKELLRRLKEIIERNQRIAKEHEYIARERKKLDPSNEKERKLLERSRRLQEESKRLLDEMAEIMRRIKKLLD

SEQ ID NO: 255 - protein, 75 aa, synthetic construct
DRQKLIEENIKLLDKHIKILEEILRLLKKDIDLLKKSSSEEVLEELKKIHRRIDKLLDESKKIHKRSSEIVKKRS

SEQ ID NO: 256 - protein, 75 aa, synthetic construct
DEQKLIETSQRLQEKSERLLEKFEQILREASDLYRKPDSEELLRRVEKLLRELEKLIRENQDLARKHEKILRDQS

SEQ ID NO: 257 - protein, 75 aa, synthetic construct
DRQELIRENIELLKKHIKIVKEIQKLIETFIELLKKSSSEEILRRLKKILKRIEKLYRESQEIHKRSEEIAKKRQ

SEQ ID NO: 258 - protein, 75 aa, synthetic construct
DEERLIDKSRELQKESEELLKELLKIFKRIEELLEKPDSEELIREIKKLLETLSEIHKRNEKLARTHEEILRQQS

SEQ ID NO: 259 - protein, 74 aa, synthetic construct
STRDVQREIAKAFKKMADVQKKLAEEIKRHVKNVEKKNKDNDEYRKIATELLKKATESQKKLKELLDRIRKSDS

SEQ ID NO: 260 - protein, 74 aa, synthetic construct
DKDDRSTSLLKRVEKLIDESDRIIDKFTTLIELSRNGKIDDDQYKKELKEILELLKKYDKHVKEVEELLKRLNS

SEQ ID NO: 261 - protein, 77 aa, synthetic construct
SKRKALEVSERVVRISEKVVRVLDESSDLLKKSYDDSDKFAELIDRHEEKIKKWKKLIKEWLEIIQRHKSSEQIDNO

SEQ ID NO: 262 - protein, 75 aa, synthetic construct
SAEEFVKLSEEAVKRSKEILDIVRKQVKLVKAGVDKHEITDSLRKSEKLIEEHKELIKTHRDLLRRENSEQIDNO

SEQ ID NO: 263 - protein, 81 aa, synthetic construct
SSTEILKRFKRALRESEKIVKHSRRVLKIIREVLKQKPTQAVHDLVRIIETQVKALEEQLKVLKRIVEALERQSSEQIDNO

SEQ ID NO: 264 - protein, 80 aa, synthetic construct
DKQKEIKDILEKTRRIAEESRKIAEKFDEIIKRSTEGKIDESLTKELEELVKEVIKLSEDDARTSDDLVRKESSEQIDNO

SEQ ID NO: 265 - protein, 81 aa, synthetic construct
DEDESIKLTRKSIEETRKSLKIIKEVVELIREVLKHIKDLDKEIFERIDKILDKYKKQVDTYDEILKEYEKKQRSEQIDNO

SEQ ID NO: 266 - protein, 70 aa, synthetic construct
SELDEQKELIKKQEKLIEEQQRLLSKIRRMFKERVKDQELLREIQKVLKRSQEIVETSKKILDRSDKTTE

SEQ ID NO: 267 - protein, 76 aa, synthetic construct
DQKEINTRIVEKLERIFKKSKEIVRQSERVISTIEKKTEDERELDLLRRHVKIVREHLKLLEELLKIIKEVQKESE

SEQ ID NO: 268 - protein, 71 aa, synthetic construct
DTEELVKRLNELLKELSKLVKEFIKILETYRKDQTKDTSKISERVDRILKTYEDLLQKYKEILEKIEKQLS

SEQ ID NO: 269 - protein, 68 aa, synthetic construct
DYARLIDQAVEVTRKVVEVNVTVARVNDKFAKHLGDEELRRVSEHLKEVSKDLQEVAKKSKDAARQVK

SEQ ID NO: 270 - protein, 77 aa, synthetic construct
DVSKVAEEYLQISKTLVDISRTLLEISERLVRLVRTVADDRSEVKKAIEDSIEVLKTSEEVVRQIKRASDKLVKAIS

SEQ ID NO: 271 - protein, 76 aa, synthetic construct
DAKEIQRRVVEIQTEVVKLQKKAVDIIRKIIEAFNNSNIDQSLLEAAKEIVKEIDKLEKLTESLLEESKKLLKRSS

SEQ ID NO: 272 - protein, 76 aa, synthetic construct
SAEEVVKLAKIFLELLRESIKLLKRSVDLLRKSSDPSLDKSEAEKVSREIEKVSDTSLKLSKKALDVVKRALKVAS

SEQ ID NO: 273 - protein, 81 aa, synthetic construct
DEKDAARKARKVSEEAKEASKKIEKALEESKRILNTLKQKKDEQEVKVIKEHEDVLRQIEKIQKQVLEIQKEVAKLLESLD

SEQ ID NO: 274 - protein, 71 aa, synthetic construct
SADDVARASEKVLRVARESAKAADKSLEVFKEVVKRGDKEAFLQVVKINEEVVKINITVIRILIEVSKTAT

SEQ ID NO: 275 - protein, 112 aa, synthetic construct
DEYVKETLKQLREALASLREADKRITELVKEARKKPLSEAARKFAEAIVTHVKVVVEHVEVVLRHVEVLVEAKKNGVIDKSILDNALRIIENVIRLLSNVIRVVDEVLQDLD

SEQ ID NO: 276 - protein, 113 aa, synthetic construct
DASDVIRRIHELFEEVHRLIEAVHRAIEDVAKAAQKKGLDESAVEILAELSKELAKLSRRLAEISREIQKVVTDPDDKEAVERLKEIIKEIKKQLDELRDRLRKLQDLLYKLK

SEQ ID NO: 277 - protein, 77 aa, synthetic construct
SEDKAHHDIVRVLEELIKIHDELMKISEEILKATSDSTATDETKEELKRRSKEAQKKSDTLVKIVKELEKESRKAQS

SEQ ID NO: 278 - protein, 73 aa, synthetic construct
DDEEKYRQIIREAQEISKTAKRILRDAQEISKRIRHQGVDRSEHQRLVDLLRELIKEHHKLLRRQQEADTRND

SEQ ID NO: 279 - protein, 74 aa, synthetic construct
DRKDKARKASEKLEEVIQRWKTVADKWKKMVDLVSNGKLSQEEVARVTEELLKIQTELAKLLEEHAKVLQESAS

SEQ ID NO: 280 - protein, 77 aa, synthetic construct
SDEESIKTQSELIKTSEELLKDVKRIDEELQKLRDDPTLDESELKKRVKEWSDRVRKAKEISRKIQEIVKESKKRSS

SEQ ID NO: 281 - protein, 72 aa, synthetic construct
DKDEELRKVIEKYREMVKEYRKVIREYEEVIKSSKTIDKSSLISLSRKMVELSQRVIDVSDEVAKVLSRKQS

SEQ ID NO: 282 - protein, 69 aa, synthetic construct
TDEERLKKQTKELKEQTKQLEKQKDLLEKISNGEISKDEIQEIIKESKKIAKESQKALDSSRKALEEVS

SEQ ID NO: 283 - protein, 75 aa, synthetic construct
DEKEVSKEIIKVLKDIAKVQQKVIEVSQRLASVLRADDDNVVKRALEEYEKILEELRELNKEIEKLTDKYRKVTS

SEQ ID NO: 284 - protein, 77 aa, synthetic construct
DSDEQTKELEKLTELHKRHVEKLKKQTKESREVDSNKLWKSKDVKDKLSESEKELQKLSDQDKKAKDALESSRRKND

SEQ ID NO: 285 - protein, 76 aa, synthetic construct
DAEEQLKLLTKLLRHQQRLLQLIKESLKLIEKIDQSSQENQDEIRKWREVTKKLRELIKTSEKLVRELEKSYKKSS

SEQ ID NO: 286 - protein, 70 aa, synthetic construct
SLRDVVRRYQELVRRYDELIKTLTEILKKYQKKGAEDKDASTELVKAVRTSLKLSKELLKLNSELLKEDS

SEQ ID NO: 287 - protein, 69 aa, synthetic construct
SKEELKRKLDELKKRSDTLKELSKKLKEISERNPDDKSVHRTIIRIHREFVKNHKEIVRVIEEIVSDKS

SEQ ID NO: 288 - protein, 71 aa, synthetic construct
SKQDEHDRLLKIHDKLVKQHDELLKLLTKLSRAGDSVTKKKLEEILRKLQEVSKQLEESLKDADKVSKDIN

SEQ ID NO: 289 - protein, 69 aa, synthetic construct
TVQSLLEQHVKIVKRSIEILERHTQILQDIARSQGVSKELEDVERQVKEYRKEVKKLEEDLRQLSRNSK

SEQ ID NO: 290 - protein, 76 aa, synthetic construct
SDSDRIEKLIRESTELLKEQQKLAKRSRELAETVESLPLTEEYLKQQREHQKKIEKLLKDSEKHLEELKRLVKSEK

SEQ ID NO: 291 - protein, 70 aa, synthetic construct
DSEKRIEDILRTDLELAKRDAELVKEHIKLVKRIDLSEELKKQVEDVEKESKKLEDSSEKLVQKVRKRSS

SEQ ID NO: 292 - protein, 80 aa, synthetic construct
DEEERAKDLRKYLEEQTQYYRTVTEHLRNLEKVVEELERRGKPSSELQQILERSQRIYKETTEIYDTSKKLIEELDKHHR

SEQ ID NO: 293 - protein, 77 aa, synthetic construct
PLEDILKRHLDKVRELVRLSEEVNKLAKEVLDILKDKRVDEKELDKVLKELEKVVEEYERAVKESRDLLRELRETTR

SEQ ID NO: 294 - protein, 75 aa, synthetic construct
DKERLLEIHERIQKLLDRNLEIIERLLRLLREARDIKDDDKLDKVIKRLKELSEESKDILDKIKELLKESEKELT

SEQ ID NO: 295 - protein, 75 aa, synthetic construct
PEDEVIRVIEELLRIAAEVDEVHRRNVEVQEEASRVTDRERLERLNRESEELIKRSRELIEEQRKLIERLERLAT

SEQ ID NO: 296 - protein, 74 aa, synthetic construct
DLEELIKEYAEVVRRHHKAVRDLERLVRELANAKHASEEELKRIATEILRIVKELIRVQERLIKLSEDSNEESR

SEQ ID NO: 297 - protein, 75 aa, synthetic construct
PTDEVIEVLKELLRIHRENLRVNEEIVEVNERASRVTDREELERLLRRSNELIKRSRELNEESKKLIEKLERLAT

SEQ ID NO: 298 - protein, 74 aa, synthetic construct
DNEEIIKEARRVVEEYKKAVDRLEELVRRAENAKHASEKELKDIVREILRISKELNKVSERLIELWERSQERAR

SEQ ID NO: 299 - protein, 72 aa, synthetic construct
PKEDIDRVSRELVRVHKELLEVLRKSTEIVEAVARNEKDERTIEEVLEEQERAVRKLEEVSKKHKEAVKRLK

SEQ ID NO: 300 - protein, 77 aa, synthetic construct
ELERLSEEIQKLSDRLIELIRRHSKVLEEIVRLLKHKDNDEREVRRLLKLLRDLTRRYEEVLRKVEEIVKRQEDESR

SEQ ID NO: 301 - protein, 72 aa, synthetic construct
PEEDILRLLRKLVEVDKELLEVVRESTEVVRLVARNEKDVETVERVLRKQEEVVRKYERVSRELEEAVRRLK

SEQ ID NO: 302 - protein, 77 aa, synthetic construct
ELKDLVEEIVKLSKENLKLWEDHSRVLEEIVRLLKHKDNDEREVRRLLKLLEDLTRRAEETSRRIEEIVKEAEDRAR

SEQ ID NO: 303 - protein, 74 aa, synthetic construct
DEERELREVLRKHHRVVREWTKVVEELKRVVELLKRGETSEEDLLRVLKKLLEMDKRILEVNREVLRVLEKRLT

SEQ ID NO: 304 - protein, 70 aa, synthetic construct
SLEEIIEELVELVRRSVEIAKESDEVARRIVESEDKKKELIDTLRDLHREWQEVTKRAEELVREAEKEVR

SEQ ID NO: 305 - protein, 72 aa, synthetic construct
TAEELLEVHKKSDRVTKEHLRVSEEILKVVEVLTRGEVSSEVLKRVLRKLEELTDKLRRVTEEQRRVVEKLN

SEQ ID NO: 306 - protein, 76 aa, synthetic construct
DLEDLLRRLRRLVDEQRRLVEELERVSRRLEKAVRDNEDERELARLSREHSDIQDKHDKLAREILEVLKRLLERTE

SEQ ID NO: 307 - protein, 72 aa, synthetic construct
PEDDVVRIIKEDLESNREVLREQKEIHRILELVTRGEVSEEAIDRVLKRQEDLLKKQKESTDKARKVVEERR

SEQ ID NO: 308 - protein, 70 aa, synthetic construct
DEVRLITEWLKLSEESTRLLKELVELTRLLRNNVPNVEEILREHERISRELERLSRRLKDLADKLERTRR

SEQ ID NO: 309 - protein, 70 aa, synthetic construct
DEDEVVKVHEEHVKSHEEIHRSHEEVVRAAEEDKRDSRELRTLMEEHRKLLEENEKSIEEVKKIHERVKR

SEQ ID NO: 310 - protein, 80 aa, synthetic construct
KKEELIDISKEVLDLDDEINKISKEILELIKKLLRLKEEGREDKDKAREVKRRIRELHRRIQELNKRLRELHKRVQETKR

SEQ ID NO: 311 - protein, 77 aa, synthetic construct
PEEDIARRVEDLLRKSEELIKESEKILKESKRLLDRNDSDKRVLETNLRLIDKHTKLLERNLELLEELLKLAEDVAK

SEQ ID NO: 312 - protein, 74 aa, synthetic construct
RFKDLSREYIEVVKRLLELSREALEVLREIKDTDKTDKKRIKELIDRLRKLIEEYKRIIDRLRKLSKDLEEEHR

SEQ ID NO: 313 - protein, 73 aa, synthetic construct
DEEELVKILKELQRLSEESLEINKRLVEILRLLRRGEVPKEEVEKKLREIKKEQEKLDREHEKIKKRIEEITK

SEQ ID NO: 314 - protein, 74 aa, synthetic construct
SLKEKILEIIERNMKLVELSNRSVEIVARILKGEKDDEETLERLLREWDKITRDYEEIIKESRKLVKELEEEAK

SEQ ID NO: 315 - protein, 74 aa, synthetic construct
SKTEILRKALEIHKEQIDIVRKLIELSEEVLKLVEESKEKNLEKLKRIDEETDRLLERLDELHKRLTELAERLK

SEQ ID NO: 316 - protein, 80 aa, synthetic construct
SDDEARKQLEEMKRRLREVEKKSKRVEERVRELERLVRENREDEDRVLKTLEDLLRENEKLVRTIERHVREQRELSKEVK

SEQ ID NO: 317 - protein, 73 aa, synthetic construct
SEEELEKKADELRKLSEEWRKLQEEDKRLSEMVEKGELDLQEVDEHSLRVLERATEVHRTVDKVIEEILRTTN

SEQ ID NO: 318 - protein, 77 aa, synthetic construct
SEKERHRESQETQEEIRRTHEEIIRKLEEILRRAKAGELPEETLDRLRRIMERLKELSERLDDLVRKLRDDHRREQK

SEQ ID NO: 319 - protein, 76 aa, synthetic construct
SEKEILEELKRILKRVKDISDRLEELDKRTEEIARREPTKELVDELVKIHRDWLRLHEEILKLVDDALKKVEDATK

SEQ ID NO: 320 - protein, 73 aa, synthetic construct
DLRELLELQREASRLHRELVKLLTELVKKLELIAKGEDIREEDLKRIKERLEEIKKRSKRIKEESDEIDKKTK

SEQ ID NO: 321 - protein, 72 aa, synthetic construct
SERELQRELNKIVRRILEIHREVSELHQRAVKLIRENDNSEELEEISRRIEELSKELEKLVREHDEIVKTIE

SEQ ID NO: 322 - protein, 77 aa, synthetic construct
SEREKLDRNDEELKEINKRVEEIKERSDRITEAIEKNERSEEEIRRLSREQNEALQRLLELHKKLVKLHRELLEDTR

SEQ ID NO: 323 - protein, 76 aa, synthetic construct
DKEDVIRVHDEQHKLIEEQLELTRRIAELVREIAKNTASEEEIKEMLKEIKRLDDRSREIQDRLQKLLEEIRRKTK

SEQ ID NO: 324 - protein, 73 aa, synthetic construct
TEEEIVELNKDIQRKSKEHIDLQNELVKKIERAIRENNITEELLEELERLLRESEKIVEEIRRITDKIRKDAK

SEQ ID NO: 325 - protein, 72 aa, synthetic construct
SEKEILERLLRLSKEQNEISEEIHRLTERLVELKRRKDDDERLKRILDRQKRLVERAREISKEYEDLLRKLE

SEQ ID NO: 326 - protein, 69 aa, synthetic construct
SMEELLRKNARLSRKQLKIIDEHLELSTKLTRGEAGDETLEEIERRSREMLEEQRRVDEESKRIREKLK

SEQ ID NO: 327 - protein, 76 aa, synthetic construct
SEEEIRDIVEKLLRTHEEVLKEIKKLLDDSERVRRRELDKKDLDRIQKEQRDIQEENKEKAKRFDELVKELKKAAK

SEQ ID NO: 328 - protein, 70 aa, synthetic construct
SEEEHRRTMEKVEKEVRDIKRRSEEVKKKVKANTLSEEDLVRLLERLVEDHKRLQDLSQEIIERDEKATK

SEQ ID NO: 329 - protein, 72 aa, synthetic construct
DEDELAKEIEDVQRRNKESQEEHDKSVKKLEAAERGEIDEDSLLRVLEEDIKVLEKDIEVLERSIEVIEKAE

SEQ ID NO: 330 - protein, 73 aa, synthetic construct
SEKELIRRLLEQQRQHLRLSERLIELSRRLVEVVRKGKDNRDLLRELKKLSEEHKKHSKDDHEKVREIREREK

SEQ ID NO: 331 - protein, 75 aa, synthetic construct
DRKDLLKRNIKLLDRHLKILDTILKLLEKLSELLKKSSSEEVVKEYKKILDEIRKLLEESKEIHKESKEILERES

SEQ ID NO: 332 - protein, 75 aa, synthetic construct
DEEKLIERSKRLQEESEQLLEKFEQILRELTELLEKPDSEELARKIKKLHDELRKIIKRNQELIREHEEILRKRD

SEQ ID NO: 333 - protein, 24 aa, synthetic construct
GEIAALEKEIAALEWEIAALEQGS

SEQ ID NO: 334 - protein, 24 aa, synthetic construct
GKIAALEYKIAALKKKIAALKQGS

SEQ ID NO: 335 - protein, 27 aa, synthetic construct
GLEQEIAALEKENAALEWEIAALEQGG

SEQ ID NO: 336 - protein, 24 aa, synthetic construct
GKIAALEYKNAALKKKIAALKQGG

SEQ ID NO: 337 - protein, 27 aa, synthetic construct
GLKQKIAALEYKNAALKKKIAALKQGG

SEQ ID NO: 338 - protein, 31 aa, synthetic construct
GKIAALKQKIAALEYKNAALKKKIAALKQGG

SEQ ID NO: 339 - protein, 24 aa, synthetic construct
GEIAALEKENAALEWEIAALEQGG

SEQ ID NO: 340 - protein, 27 aa, synthetic construct
GLKQKIAALEYKNAALKKKIAALKQGS

SEQ ID NO: 341 - protein, 31 aa, synthetic construct
GKIAALKQKIAALEYKNAALKKKIAALKQGS

SEQ ID NO: 342 - protein, 31 aa, synthetic construct
GEIAALEQEIAALEKENAALEWEIAALEQGG

SEQ ID NO: 343 - protein, 21 aa, synthetic construct
EIAALEKEIAALEKEIAALEK

SEQ ID NO: 344 - protein, 21 aa, synthetic construct
KIAALKEKIAALKEKIAALKE

SEQ ID NO: 345 - protein, 28 aa, synthetic construct
EIAALEKEIAALEKEIAALEKEIAALEK

SEQ ID NO: 346 - protein, 28 aa, synthetic construct
KIAALKEKIAALKEKIAALKEKIAALKE

SEQ ID NO: 347 - protein, 21 aa, synthetic construct
EISALEKEISALEKEISALEK

SEQ ID NO: 348 - protein, 21 aa, synthetic construct
KISALKEKISALKEKISALKE

SEQ ID NO: 349 - protein, 28 aa, synthetic construct
EISALEKEISALEKEISALEKEISALEK

SEQ ID NO: 350 - protein, 28 aa, synthetic construct
KISALKEKISALKEKISALKEKISALKE

SEQ ID NO: 351 - protein, 21 aa, synthetic construct
EVAALEKEVAALEKEVAALEK

SEQ ID NO: 352 - protein, 21 aa, synthetic construct
KVAALKEKVAALKEKVAALKE

SEQ ID NO: 353 - protein, 28 aa, synthetic construct
EVAALEKEVAALEKEVAALEKEVAALEK

SEQ ID NO: 354 - protein, 28 aa, synthetic construct
KVAALKEKVAALKEKVAALKEKVAALKE

SEQ ID NO: 355 - protein, 21 aa, synthetic construct
EVSALEKEVSALEKEVSALEK

SEQ ID NO: 356 - protein, 21 aa, synthetic construct
KVSALKEKVSALKEKVSALKE

SEQ ID NO: 357 - protein, 28 aa, synthetic construct
EVSALEKEVSALEKEVSALEKEVSALEK

SEQ ID NO: 358 - protein, 28 aa, synthetic construct
KVSALKEKVSALKEKVSALKEKVSALKE

SEQ ID NO: 359 - protein, 30 aa, synthetic construct
AQLEKELQALEKENAQLEWELQALEKELAQ

SEQ ID NO: 360 - protein, 30 aa, synthetic construct
AQLKKKLQALKKKNAQLKWKLQALKKKLAQ

SEQ ID NO: 361 - protein, 14 aa, synthetic construct
KIAALKQKIASLKQ

SEQ ID NO: 362 - protein, 14 aa, synthetic construct
EIDALEYENDALEQ

SEQ ID NO: 363 - protein, 14 aa, synthetic construct
KIRRLKQKNARLKQ

SEQ ID NO: 364 - protein, 14 aa, synthetic construct
EIAALEYEIAALEQ

SEQ ID NO: 365 - protein, 21 aa, synthetic construct
EIRRLEEEIRRLEEENRRLEY

SEQ ID NO: 366 - protein, 21 aa, synthetic construct
RNEELRYRIEELRRRIEELRR

SEQ ID NO: 367 - protein, 28 aa, synthetic construct
EIRRLEEEIRRLEEEIRRLEEENRRLEY

SEQ ID NO: 368 - protein, 28 aa, synthetic construct
RIEELRRRNEELRYRIEELRRRIEELRR

SEQ ID NO: 369 - protein, 21 aa, synthetic construct
EIKKLEEEIKKLEEENKKLEY

SEQ ID NO: 370 - protein, 21 aa, synthetic construct
KNEELKYKIEELKKKIEELKK

SEQ ID NO: 371 - protein, 28 aa, synthetic construct
EIKKLEEEIKKLEEEIKKLEEENKKLEY

SEQ ID NO: 372 - protein, 28 aa, synthetic construct
KIEELKKKNEELKYKIEELKKKIEELKK

SEQ ID NO: 373 - protein, 36 aa, synthetic construct
KVSALKEKVSALKEKVSALKEKVSALKEKVSALKEG

SEQ ID NO: 374 - protein, 37 aa, synthetic construct
EVSALKEKVSALEKEVSALKEKVSALEKEVSALEKGG

SEQ ID NO: 375 - protein, 5 aa, synthetic construct
RRRGN

SEQ ID NO: 376 - protein, 23 aa, synthetic construct
RGKRGGHATNYRIVAPRSRDERG

SEQ ID NO: 377 - protein, 23 aa, synthetic construct
ESLSAIEAELEKVAHQLQALRRG

SEQ ID NO: 378 - protein, 23 aa, synthetic construct
KISGGPHISYPPLHEWVLREGEE

SEQ ID NO: 379 - protein, 23 aa, synthetic construct
KISGGPHISYPPLHEWVLREGAA

SEQ ID NO: 380 - protein, 24 aa, synthetic construct
LRELDLAQEKTGWFALGSRSHGTL

SEQ ID NO: 381 - protein, 24 aa, synthetic construct
LRELDLAQEKTGWFALGSRHCGRT

SEQ ID NO: 382 - protein, 23 aa, synthetic construct
YKKAGSGIPLRMNSLFRKRNKGK

SEQ ID NO: 383 - protein, 23 aa, synthetic construct
VFSDLKNESGQEDKKRLLLGLDR

SEQ ID NO: 384 - protein, 22 aa, synthetic construct
VFSDLKNESGQEDKKRLLLGLD

SEQ ID NO: 385 - protein, 23 aa, synthetic construct
DGEDEGYFQELLGSVNSLLKELR

SEQ ID NO: 386 - protein, 22 aa, synthetic construct
DGEDEGYFQELLGSVNSLLKEL

SEQ ID NO: 387 - protein, 23 aa, synthetic construct
LEKEKLQARQQQLLKKIEHARTC

SEQ ID NO: 388 - protein, 20 aa, synthetic construct
LEKEKLQARQQQLLKKIEHA

SEQ ID NO: 389 - protein, 23 aa, synthetic construct
GPGPNGQRFCINSAALKFKPRKH

SEQ ID NO: 390 - protein, 23 aa, synthetic construct
LRNAQMKNAIRKLCSRKAISSVK

SEQ ID NO: 391 - protein, 23 aa, synthetic construct
LRNAQMKNAIRKLCSRKAISSDK

SEQ ID NO: 392 - protein, 23 aa, synthetic construct
RRSCNDCQQMIVKLGGGTAIAAS

SEQ ID NO: 393 - protein, 23 aa, synthetic construct
RRSCNDCQQMIVKLGGGTAIADS

SEQ ID NO: 394 - protein, 23 aa, synthetic construct
QKSRKVDSKSLFHYRQYSAGKAA

SEQ ID NO: 395 - protein, 23 aa, synthetic construct
QKSRKVDSKSLFHYRQYSAGKDD

SEQ ID NO: 396 - protein, 28 aa, synthetic construct
QKSRKVDSKSLFHYRQYSAGKAAKASTN

SEQ ID NO: 397 - protein, 23 aa, synthetic construct
REIQGIFFKEDSHKESNDCSCGG

SEQ ID NO: 398 - protein, 23 aa, synthetic construct
SLTDVALEHHEECDCVCRGSTGG

SEQ ID NO: 399 - protein, 23 aa, synthetic construct
RRLDGKEEDEKMSRASDRFRGLR

SEQ ID NO: 400 - protein, 19 aa, synthetic construct
TRGVEEVAEGVVLLRRRGN

SEQ ID NO: 401 - protein, 5 aa, synthetic construct
NLGIR

SEQ ID NO: 402 - protein, 10 aa, synthetic construct
QRKLQRTSRG

SEQ ID NO: 403 - protein, 10 aa, synthetic construct
QRKLQRTSRA

SEQ ID NO: 404 - protein, 12 aa, synthetic construct
PHKRLLKGSQYG

SEQ ID NO: 405 - protein, 5 aa, synthetic construct
  REGION >5: note = The entire sequence of amino acids 1-5 can be repeated one or more times.
GSGGS

SEQ ID NO: 406 - protein, 4 aa, synthetic construct
  REGION >4: note = The entire sequence of amino acids 1-4 can be repeated one or more times.
GGGS

SEQ ID NO: 407 - protein, 4 aa, synthetic construct
GGSG

SEQ ID NO: 408 - protein, 5 aa, synthetic construct
GGSGG

SEQ ID NO: 409 - protein, 5 aa, synthetic construct
GSGSG

SEQ ID NO: 410 - protein, 5 aa, synthetic construct
GSGGG

SEQ ID NO: 411 - protein, 5 aa, synthetic construct
GGGSG

SEQ ID NO: 412 - protein, 5 aa, synthetic construct
GSSSG

SEQ ID NO: 413 - protein, 55 aa, synthetic construct
SIAATLENDLARLENENARLERDIANLERDLARLEREEAYFGSGSGSGSGSRRRG